SEQUENCE LISTING (3 sequences)

SEQ ID NO: 1 - DNA, 2737 bp, Homo sapiens
ggagcccggagcccgccttcggagctacggcctaacggcggcggcgactgcagtctggagggtccacacttgtgattctcaatggagagtgaaaacgcagattcataatgaaaactagcccccgtcggccactgattctcaaaagacggaggctgccccttcctgttcaaaatgccccaagtgaaacatcagaggaggaacctaagagatcccctgcccaacaggagtctaatcaagcagaggcctccaaggaagtggcagagtccaactcttgcaagtttccagctgggatcaagattattaaccaccccaccatgcccaacacgcaagtagtggccatccccaacaatgctaatattcacagcatcatcacagcactgactgccaagggaaaagagagtggcagtagtgggcccaacaaattcatcctcatcagctgtgggggagccccaactcagcctccaggactccggcctcaaacccaaaccagctatgatgccaaaaggacagaagtgaccctggagaccttgggaccaaaacctgcagctagggatgtgaatcttcctagaccacctggagccctttgcgagcagaaacgggagacctgtgcagatggtgaggcagcaggctgcactatcaacaatagcctatccaacatccagtggcttcgaaagatgagttctgatggactgggctcccgcagcatcaagcaagagatggaggaaaaggagaattgtcacctggagcagcgacaggttaaggttgaggagccttcgagaccatcagcgtcctggcagaactctgtgtctgagcggccaccctactcttacatggccatgatacaattcgccatcaacagcactgagaggaagcgcatgactttgaaagacatctatacgtggattgaggaccactttccctactttaagcacattgccaagccaggctggaagaactccatccgccacaacctttccctgcacgacatgtttgtccgggagacgtctgccaatggcaaggtctccttctggaccattcaccccagtgccaaccgctacttgacattggaccaggtgtttaagcagcagaaacgaccgaatccagagctccgccggaacatgaccatcaaaaccgaactccccctgggcgcacggcggaagatgaagccactgctaccacgggtcagctcatacctggtacctatccagttcccggtgaaccagtcactggtgttgcagccctcggtgaaggtgccattgcccctggcggcttccctcatgagctcagagcttgcccgccatagcaagcgagtccgcattgcccccaaggtgctgctagctgaggaggggatagctcctctttcttctgcaggaccagggaaagaggagaaactcctgtttggagaagggttttctcctttgcttccagttcagactatcaaggaggaagaaatccagcctggggaggaaatgccacacttagcgagacccatcaaagtggagagccctcccttggaagagtggccctccccggccccatctttcaaagaggaatcatctcactcctgggaggattcgtcccaatctcccaccccaagacccaagaagtcctacagtgggcttaggtccccaacccggtgtgtctcggaaatgcttgtgattcaacacagggagaggagggagaggagccggtctcggaggaaacagcatctactgcctccctgtgtggatgagccggagctgctcttctcagaggggcccagtacttcccgctgggccgcagagctcccgttcccagcagactcctctgaccctgcctcccagctcagctactcccaggaagtgggaggaccttttaagacacccattaaggaaacgctgcccatctcctccaccccgagcaaatctgtcctccccagaacccctgaatcctggaggctcacgcccccagccaaagtagggggactggatttcagcccagtacaaacctcccagggtgcctctgaccccttgcctgaccccctggggctgatggatctcagcaccactcccttgcaaagtgctcccccccttgaatcaccgcaaaggctcctcagttcagaacccttagacctcatctccgtcccctttggcaactcttctccctcagatatagacgtccccaagccaggctccccggagccacaggtttctggccttgcagccaatcgttctctgacagaaggcctggtcctggacacaatgaatgacagcctcagcaagatcctgctggacatcagctttcctggcctggacgaggacccactgggccctgacaacatcaactggtcccagtttattcctgagctacagtagagccctgcccttgcccctgtgctcaagctgtccaccatcccgggcactccaaggctcagtgcaccccaagcctctgagtgaggacagcaggcagggactgttctgctcctcatagctccctgctgcctgattatgcaaaagtagcagtcacaccctagccactgctgggaccttgtgttccccaagagtatctgattcctctgctgtccctgccaggagctgaagggtgggaacaacaaaggcaatggtgaaaagagattaggaaccccccagcctgtttccattctctgcccagcagtctcttaccttccctgatctttgcagggtggtccgtgtaaatagtataaattctccaaattatcctctaattataaatgtaagct

SEQ ID NO: 2 - protein, 748 aa, homo sapiens
MKTSPRRPLILKRRRLPLPVQNAPSETSEEEPKRSPAQQESNQAEASKEVAESNSCKFPAGIKIINHPTMPNTQVVAIPNNANIHSIITALTAKGKESGSSGPNKFILISCGGAPTQPPGLRPQTQTSYDAKRTEVTLETLGPKPAARDVNLPRPPGALCEQKRETCADGEAAGCTINNSLSNIQWLRKMSSDGLGSRSIKQEMEEKENCHLEQRQVKVEEPSRPSASWQNSVSERPPYSYMAMIQFAINSTERKRMTLKDIYTWIEDHFPYFKHIAKPGWKNSIRHNLSLHDMFVRETSANGKVSFWTIHPSANRYLTLDQVFKQQKRPNPELRRNMTIKTELPLGARRKMKPLLPRVSSYLVPIQFPVNQSLVLQPSVKVPLPLAASLMSSELARHSKRVRIAPKVLLAEEGIAPLSSAGPGKEEKLLFGEGFSPLLPVQTIKEEEIQPGEEMPHLARPIKVESPPLEEWPSPAPSFKEESSHSWEDSSQSPTPRPKKSYSGLRSPTRCVSEMLVIQHRERRERSRSRRKQHLLPPCVDEPELLFSEGPSTSRWAAELPFPADSSDPASQLSYSQEVGGPFKTPIKETLPISSTPSKSVLPRTPESWRLTPPAKVGGLDFSPVQTSQGASDPLPDPLGLMDLSTTPLQSAPPLESPQRLLSSEPLDLISVPFGNSSPSDIDVPKPGSPEPQVSGLAANRSLTEGLVLDTMNDSLSKILLDISFPGLDEDPLGPDNINWSQFIPELQ

SEQ ID NO: 3 - RNA, 6 nt, Artificial
  Eukaryotic transcription termination signal
aauaaa

(19) United States
(12) Patent Application Publication
(10) Pub. No.: US 2004/0109844 A1
(43) Pub. Date: Jun. 10, 2004

(54) Methods of treating age-related defects and diseases

(75) Inventors: Robert H. Costa, Oak Park, IL (US); Xinhe Wang, Upper Arlington, OH (US); Yongjun Tan, Arlington Heights, IL (US); Vladimir Kalinichenko, Clarendon Hills, IL (US); Katherine Krupczak-Hollis, Chicago, IL (US); I-Ching Wang, Chicago, IL (US); Michael Major, Chicago, IL (US)

Correspondence Address: Jason J. Derry, McDonnell Boehnen Hulbert & Berghoff, 32nd Floor, 300 S. Wacker Drive, Chicago, IL, 60606, US

(73) Assignee: The Board of Trustees of the University of Illinois

(21) Appl. No.: 10/650,609

(22) Filed: Aug. 28, 2003

Related U.S. Application Data
(60) Provisional application No. 60/406,582, filed on Aug. 28, 2002.
(60) Provisional application No. 60/426,068, filed on Nov. 13, 2002.

(51) Int. Cl. A61K 48/00; A61K 38/18; A61K 38/19; A61K 38/20

(52) U.S. Cl. 424/85.1; 424/85.2; 514/12; 514/44

(57) ABSTRACT

The invention provides a method of preventing or amelioriating age-related proliferation defects or age-related diseases in a patient by stimulating FoxM1B protein expression, nuclear localization, or both protein expression and nuclear localization of FoxM1B or by introducing a recombinant nucleic acid construct that comprises a truncated FoxM1B gene into target cells, thereby restoring proliferative potential of the target cells. The invention also provides a method of treating diseases or disorders associated with premature aging. The invention further provides a method of treating or ameliorating lung damage. Specifically, the methods of the invention comprise inducing expression and nuclear localization of FoxM1B protein in a target cell by contacting the cell with a growth factor or a cytokine.

This application is related to and claims priority to U.S. provisional application Serial No. 60/406,582 filed Aug. 28, 2002 and U.S. provisional application Serial No. 60/426,068 filed Nov. 13, 2002, the disclosure of each of which is incorporated by reference herein.

This application was supported by a Public Service grant from the National Institutes of Diabetes and Digestive and Kidney Diseases, grant number DK54687 and the National Institute on Aging, grant number AG21842. The U.S. government may have certain rights to this invention.

### BACKGROUND OF THE INVENTION

1. Field of the Invention

The invention relates to methods for treating and preventing symptoms associated with aging by inducing expression and/or nuclear localization of protein in select target cells. The invention also relates to methods of treating age-related diseases and age-related proliferation disorders, as well as methods of treating diseases or disorders associated with premature aging by inducing expression and nuclear localization of FoxM1B protein. The invention particularly relates to methods of inducing FoxM1B protein expression and inducing or facilitating translocation of FoxM1B protein to the nucleus of a target cell, where it potentiates transcription of many essential cell cycle promotion genes, thereby restoring cell proliferation. Specifically, the invention relates to methods of preventing or ameliorating age-related disorders or diseases and diseases or disorders associated with premature aging comprising administering to a patient a therapeutically effective amount of growth hormone.

2. Background of the Related Art

An underlying mechanism of the aging process involves reduced cellular proliferation and repair in response to tissue injury. During the aging process, the expression patterns of several genes involved in regulating the cell cycle become altered. These defects in the mitotic machinery contribute to chromosome instability and mutations that lead to many diseases found in the elderly (Ly et al., 2000, Science 287: 2486-2492). The mechanisms involved in the progressive decline in cellular proliferation with aging remain mostly uncharacterized, however.

The Forkhead box transcription factors have been implicated in regulating cellular proliferation and longevity, particularly the Forkhead Box M1B (FoxM1B) transcription factor (also known as Trident and HFH-11B). For example, increased longevity was found in the nematode C. elegans bearing a mutant daf-2 gene, which encodes the worm homolog of the insulin/Insulin-like Growth Factor 1 (IGF1) receptor (Lin et al., 1997, Science 278:1319-1322; Ogg et al., 1997, Nature 389:994-999). Disruption of the daf-2 gene abolishes insulin-mediated activation of the phosphatidylinositol 3-kinase (P13K)-protein kinase B/Akt (Akt) signal transduction pathway and prevents inhibition of the forkhead transcription factor daf-16 (corresponding to mammalian homologs FoxO1 or Fkhr) (Paradis and Ruvkun, 1998, Genes Dev. 12:2488-2498). Activation of the P13K/Akt pathway phosphorylates the C-terminus of the Daf-16 (FoxO1; Fkhr) gene product and mediates its nuclear export into the cytoplasm, thus preventing FoxO1 transcriptional activation of target genes (Biggs et al., 1999, Proc. Natl. Acad. Sci. USA 96:7421-7426; Brunet et al., 1999, Cell 96:857-68; Guo et al., 1999, J. Biol. Chem. 274:17184-17192).

More recent studies of Daf-2° C. elegans mutants have demonstrated that Daf-16 stimulates expression of genes that limit oxidative stress (Barsyte et al., 2001, FASEB J. 15:627-634; Honda et al., 1999, FASEB J. 13:1385-1393; Wolkow et al., 2000, Science 290:147-150) and that the mammalian FoxO1 gene could functionally replace the Daf-16 gene in C. elegans (Lee et al., 2001, Curr. Biol. 11:1950-1957). In proliferating mammalian cells, the P13K/Akt signal transduction pathway is essential for G1 to S-phase progression because it prevents transcriptional activity of the FoxO1 and FoxO3 proteins, which stimulate expression of the CDK inhibitor p27^{kip1} gene (Medema et al., 2000, Nature 404:782-787). Moreover, genetic studies in budding yeast demonstrated that forkhead Fkh1 and Fkh2 proteins are components of a transcription factor complex, which regulates expression of genes critical for progression into mitosis (Hollenhorst et al., 2001, Genes Dev. 15:2445-2456; Koranda et al., 2000, Nature 406:94-98; Kumar et al., 2000, Curr. Biol. 10:896-906; Pic et al., 2000, EMBO J. 19:3750-3761).

FoxM1B is a proliferation-specific transcription factor that shares 39% amino acid homology with the HNF-3 winged helix DNA binding domain. The molecule also contains a potent C-terminal transcriptional activation domain that possesses several phosphorylation sites for M-phase specific kinases as well as PEST sequences that mediate rapid protein degradation (Korver et al., 1997, Nucleic Acids Res. 25:1715-1719; Korver et al., 1997, Genomics 46:435-442; Yao et al., 1997, J. Biol. Chem. 272:19827-19836; Ye et al., 1997, Mol. Cell Biol. 17:1626-1641).

FoxM1B is expressed in several tumor-derived epithelial cell lines and is induced by serum prior to the G₁/S transition (Korver et al., 1997, Nucleic Acids Res. 25: 1715-1719; Korver et al., 1997, Genomics 46: 435-442; Yao et al., 1997, J. Biol. Chem. 272: 19827-19836; Ye et al., 1997, Mol. Cell Biol. 17: 1626-1641). In situ hybridization studies show that FoxM1B is expressed in embryonic liver, intestine, lung, and renal pelvis (Ye et al., 1997, Mol. Cell Biol. 17: 1626-1641). In adult tissue, however, FoxM1B is not expressed in postmitotic, differentiated cells of the liver and lung, although it is expressed in proliferating cells of the thymus, testis, small intestine, and colon (Id). FoxM1B expression is reactivated in the liver prior to hepatocyte DNA replication following regeneration induced by partial hepatectomy (Id).

Micro-array analysis showed that diminished proliferation exhibited by fibroblasts from either elderly patients or genetically aged patients with Hutchinson-Gilford progeria is associated with reduced expression of Cyclin F, Cyclin A, Cyclin B, Cdc25B and p55Cdc expression, as well as a decline in FoxM1B levels. These studies indicated that an underlying mechanism of the aging process involves defective induction of cell cycle promotion genes and dysfunction of the mitotic machinery. These proliferation defects ultimately result in chromosome instability and mutations leading to a variety of diseases found in the elderly population. As described herein, restoring only FoxM1B expression in regenerating liver of old transgenic mice is sufficient to stimulate expression of these diminished cell cycle promoting genes and restores levels of hepatocyte progression into DNA synthesis and mitosis similar to those found in young regenerating mouse liver.

Since all proliferating cells display induced expression of FoxM1B, restoring FoxM1B expression in a variety of distinct cell types is likely to potentiate transcription of the cell cycle promotion genes and alleviate the proliferation defects observed during the aging process. Thus, restoring FoxM1B expression in proliferating cells of elderly patients will be efficacious in alleviating defective cellular proliferation observed with the aging process. In addition, maintaining FoxM1B levels in all cell types will prevent diminished cellular proliferation associated with aging.

### SUMMARY OF THE INVENTION

Diminished expression of the proliferation-specific Forkhead Box M1B (FoxM1B) transcription factor is associated with reduction in both cellular proliferation and expression of cell cycle progression genes during aging. Liver regeneration studies as described herein demonstrate that maintaining FoxM1B expression in hepatocytes from 12-month old (old-aged) transgenic (TG) mice increase hepatocyte proliferation to levels similar to those observed in young regenerating mouse liver. Also, maintaining FoxM1B levels in old-aged proliferating cells is associated with increased expression of numerous genes required for progression into S-phase and mitosis. Collectively, these results suggest that FoxM1B modulates the transcriptional network of genes essential for cellular proliferation and that its reduced expression contributes to the decline in cellular proliferation characteristic of hepatocytes and other cells during aging.

The invention provides a method for treating and preventing symptoms associated with aging comprising the step of inducing nuclear localization or inducing expression and nuclear localization of FoxM1B protein in target cells. In certain aspects, the symptoms can be associated with the effects of normal aging or of premature aging.

The invention also provides a method of preventing or amelioriating the effects of an age-related disease or age-related proliferation disorder in a patient comprising the step of inducing expression and/or nuclear localization of FoxM1B protein in a target cell affected by the age-related disease or disorder. The invention also provides a method of treating diseases or disorders associated with premature aging comprising the step of inducing expression and/or nuclear localization of FoxM1B protein in a target cell affected by the age-related disease or disorder.

In particular aspects, the methods of the invention induce expression and/or nuclear localization of FoxM1B protein in a target cell, the methods comprising the step of contacting the target cell with a growth factor or a cytokine. For example, a growth factor can be human growth hormone, hepatocyte growth factor, epidermal growth factor, transforming growth factor α, or a growth factor that induces Ras-MAP kinase signaling. A cytokine can be, for example, tumor necrosis factor α, interleukin 6 (IL-6), IL-1α, or IL-1β. A target cell can express FoxM1B endogenously or can be engineered to express or over-express FoxM1B protein, or FoxM1B expression can be induced in the cell. Methods for selecting the target cell are described below. Preferably, a target cell is a mammalian cell.

The invention also provides target cells, preferably mammalian target cells, into which have been introduced a recombinant nucleic acid construct of the invention. In a particular aspect, the recombinant nucleic acid construct of the invention comprises SEQ ID NO: 1. In preferred embodiments, the cells are intestinal or colonic epithelial cells, thymocytes in the thymus and lymphocytes in the spleen, or basal cells of the skin. Such cells can be used in therapeutic methods as described herein. For example, a recombinant nucleic acid construct of the invention, preferably comprising SEQ ID NO: 1, can be introduced into a target cell ex vivo or in vivo to restore proliferative potential of the target cell.

In addition, the invention provides a method of preventing or amelioriating the effects of lung injury comprising the step of inducing expression and/or nuclear localization of FoxM1B protein in a lung cell. In a particular aspect, the invention provides methods of stimulating lung regeneration in lung cells that express FoxM1B protein by inducing FoxM1B protein to translocate into the nucleus of the lung cells. In a particular aspect, the methods of the invention comprise inducing expression and/or nuclear localization of FoxM1B protein in lung cells by contacting the cells with a growth factor, a cytokine, or a compound identified in a screening method as described herein.

The invention further provides methods of preventing or ameliorating lung damage in a mammal comprising the steps of introducing target cells, that express FoxM1B protein, into the mammal and thereafter contacting the target cells with a growth factor or a cytokine or another compound identified in a screening method as described herein. In this aspect, target cells are removed from an individual and reintroduced into a recipient individual, most preferably the same individual to minimize immunological complications. In preferred embodiments, the target cells express FoxM1B endogenously. In another preferred embodiment, the target cells are contacted ex vivo with a recombinant nucleic acid construct of the invention whereby the cells express FoxM1B protein. Preferably, the recombinant nucleic acid construct comprises SEQ ID NO: 1. Both allografts and autografts as disclosed herein are contemplated by the invention to protect or ameliorate tissue damage or disease in a patient. The invention provides these methods wherein the target cells removed from an individual are contacted with a growth factor or a cytokine or a compound identified in a screening method as described herein that induces expression, nuclear localization or expression and nuclear localization of FoxM1B protein prior to or after introducing the cells into a recipient.

In another aspect, a method of the invention can be used for treating an individual who suffers from an age-related disease or proliferation disorder, lung damage, or a disorder associated with premature aging. In this aspect, the methods of the invention prevent further damage or disease progression or reverses damage or disease progression. The methods of the invention can also be applied to an individual awaiting an organ or tissue transplant or to an organ or tissue removed from a donor to be transplanted into a recipient.

In addition, the invention provides screening methods for identifying compounds that prevent or amelioriate the effects of an age-related disease or age-related proliferation disorder in a patient. In one aspect, a method of screening for such compounds comprises the steps of: contacting a plurality of cells with a candidate compound, wherein the cells comprise a full-length or less than full-length FoxM1B gene, but do not express FoxM1B protein under conventional culture conditions; assaying FoxM1B localization in the cells; and identifying a candidate compound when FoxM1B is localized in the nuclei of cells contacted with the compound but not localized in the nuclei of cells not contacted with the compound. In another aspect, such methods comprise the steps of: contacting a plurality of cells with a candidate compound, wherein the cells comprise a full-length or less than full-length FoxM1B gene, but do not express FoxM1B protein under conventional culture conditions; assaying expression of cyclin kinase inhibitors p21^{Cip1} (p21) and p27^{Kip1} (p27), and the mitosis promoting cdc25B phosphatase in the cells; and identifying a candidate compound when p21 and p27 protein levels are decreased while cdc25B protein levels are increased in cells contacted with the compound compared with cells not contacted with the compound.

The invention also provides screening methods for identifying compounds that induce lung regeneration. In one aspect, such methods comprise the steps of: contacting a plurality of cells with a candidate compound, wherein the cells comprise the full-length or less than full-length FoxM1B gene, but do not express FoxM1B protein under conventional culture conditions; assaying FoxM1B localization in the cells; selecting a candidate compound when FoxM1B expression is induced and FoxM1B protein is localized in the nuclei of cells contacted with the compound but not localized in the nuclei of cells not contacted with the compound; and identifying a compound as a compound that can induce lung regeneration when lung cells are induced to proliferate when contacted with the compound in vitro or in vivo. In a particular aspect, the lung cells are primary lung cells.

The invention further provides screening methods for identifying compounds that induce nuclear localization of FoxM1B protein. In one aspect, such methods comprise the steps of: contacting a cell with a compound, wherein the cell expresses a green fluorescent protein-FoxM1B (GFP-FoxM1B) fusion protein; detecting localization of the GFP-FoxM1B protein in the cells; and identifying a compound that induces FoxM1B localization if the GFP-FoxM1B protein is localized in the nuclei of the cells. In another aspect, such methods of screening for compounds that induce nuclear localization of FoxM1B protein, comprise the steps of: contacting a transgenic mouse with a compound, wherein at least an identifiable portion of the cells of the transgenic mouse express a green fluorescent protein-FoxM1B (GFP-FoxM1B) fusion protein; detecting localization of the GFP-FoxM1B protein in a cell comprising a GFP-fusion protein-encoding nucleic acid that is removed from the mouse; and identifying a compound that induces FoxM1B localization if the GFP-FoxM1B protein is localized in the nuclei of the cells.

The invention further provides methods for treating or preventing symptoms associated with aging comprising the step of introducing a recombinant nucleic acid construct that comprises SEQ ID NO: 1 into target cells, thereby restoring proliferative potential of the target cells; methods for preventing or amelioriating the effects of an age-related disease or age-related proliferation disorder in a patient comprising the step of introducing a recombinant nucleic acid construct that comprises SEQ ID NO: 1 into target cells, thereby restoring proliferative potential of the target cells; methods for treating diseases or disorders associated with premature aging comprising the step of introducing a recombinant nucleic acid construct that comprises SEQ ID NO: 1 into the target cell, thereby restoring proliferative potential of the target cells; and methods for preventing or ameliorating the effects of lung injury comprising the step of introducing a recombinant nucleic acid construct that comprises SEQ ID NO: 1 into lung cells, thereby restoring proliferative potential of the lung cells. In one aspect, such methods of the invention further comprise the step of inducing FoxM1B expression and/or nuclear localization in the target cell or lung cell by contacting the cells with a growth factor or a cytokine, as described herein.

Specific preferred embodiments of the invention will become evident from the following more detailed description of certain preferred embodiments and the claims.

### DETAILED DESCRIPTION OF THE DRAWINGS

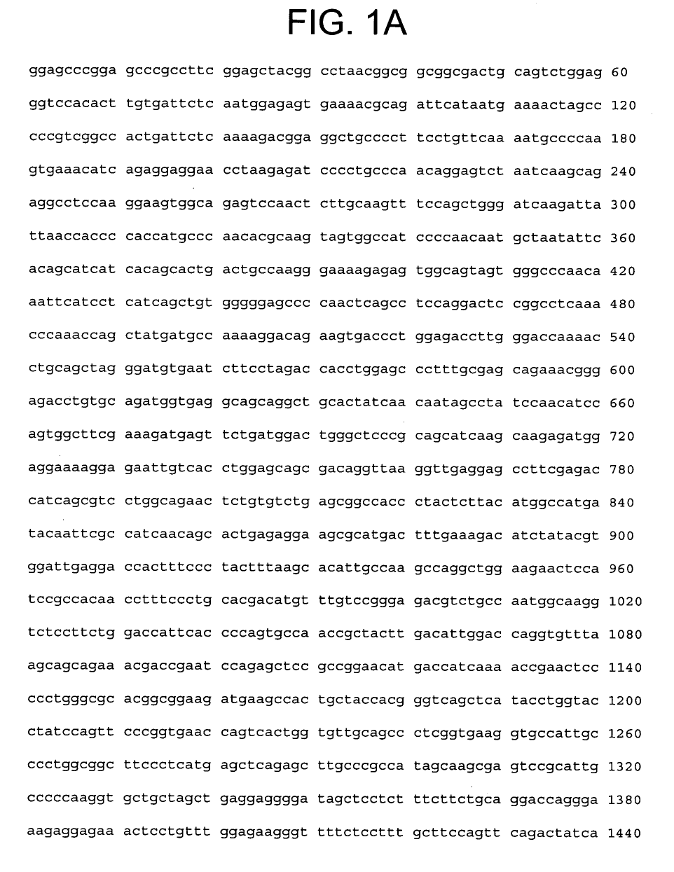

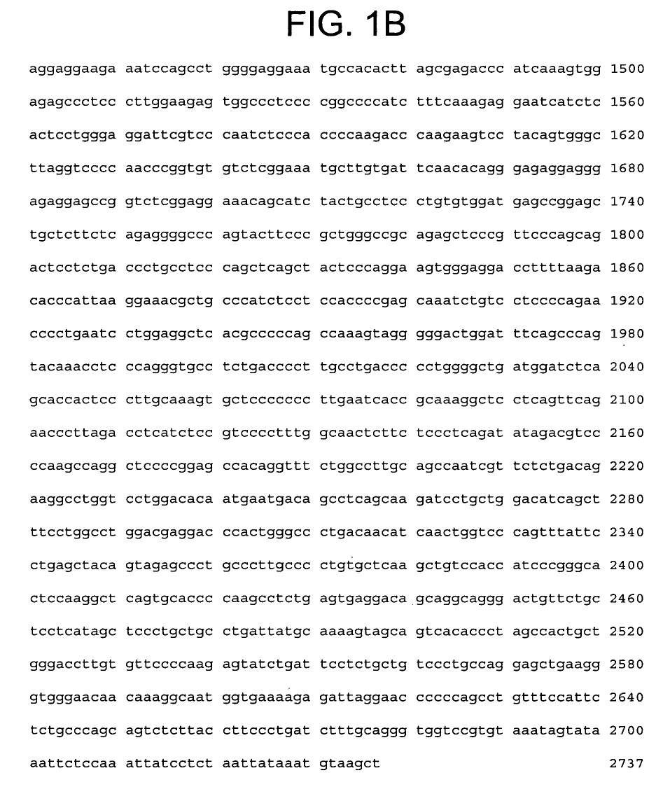

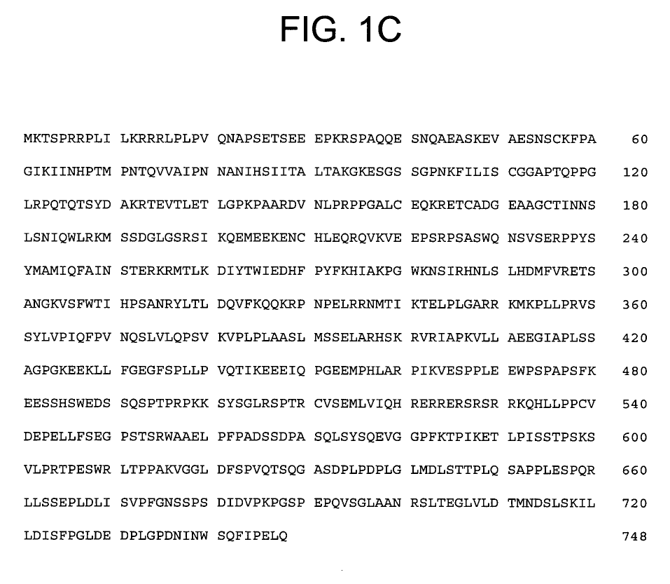

FIGS. 1A-B depicts the human FoxM1B cDNA comprising a deletion of the terminal 972 nucleotides at the 3′ end of the sequence (SEQ ID NO: 1).

FIG. 1C depicts the human FoxM1B protein sequence (SEQ ID NO: 2) encoded by the nucleotide sequence as set forth in SEQ ID NO: 1.

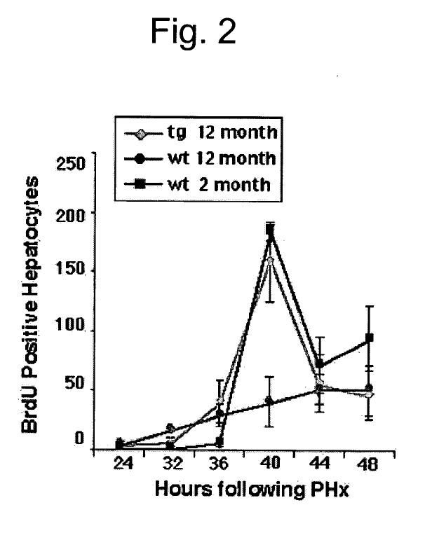

FIG. 2 shows a graph representing 5-bromo-2′-deoxy-uridine (BrdU) incorporation (as a measure of DNA replication) at the indicated hours after partial hepatectomy (PHx) in twelve month old wild type CD-1 mice (WT, solid circles), twelve month old transgenic CD-1 mice (TG, solid diamonds), or two month old wild type CD-1 mice (solid squares).

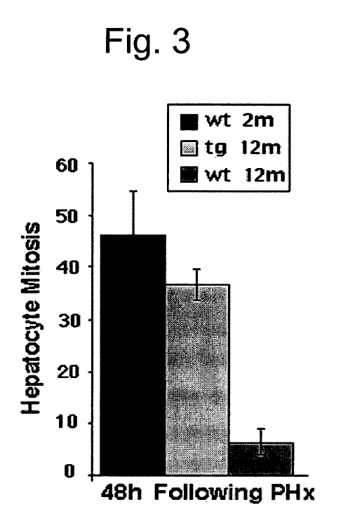

FIG. 3 shows a graph representing increased hepatocyte mitosis in regenerating livers of old-aged TG mice at 48 hours post PHx.

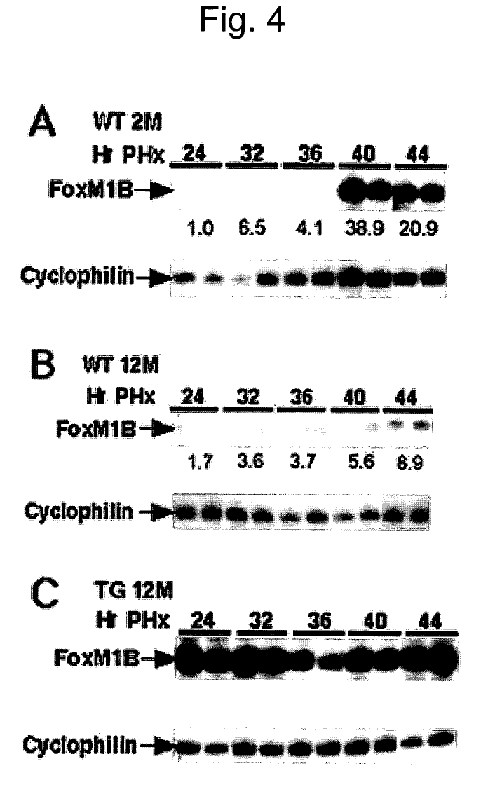

FIGS. 4A through 4C shows RNase protection assays performed using total RNA isolated at the indicated hours post PHx from regenerating liver of two-month-old WT mice (FIG. 4A), twelve-month-old WT mice (FIG. 4B), and twelve month old TG mice (FIG. 4C).

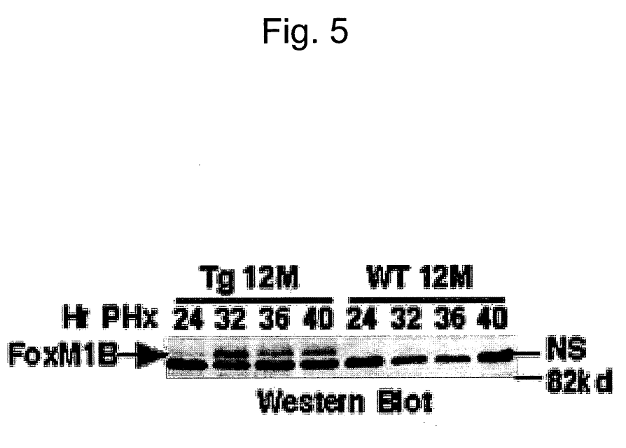

FIG. 5 shows the results of a western blot analysis using anti-FoxM1B antibodies performed with total liver protein extracts isolated from regenerating livers of twelve month old WT and TG mice at the indicated time points. FoxM1B protein migrates more slowly than a non-specific (NS) band also detected.

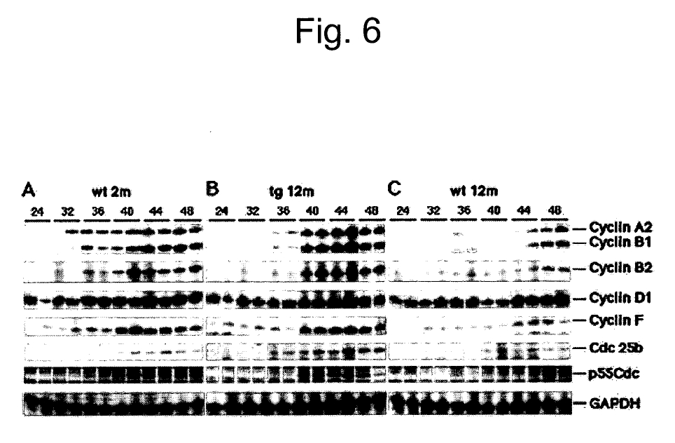

FIG. 6 shows an RNase protection assay demonstrating increased expression of cell cycle promotion genes in regenerating liver of old TG mice compared with WT mice at the indicated hours following PHx.

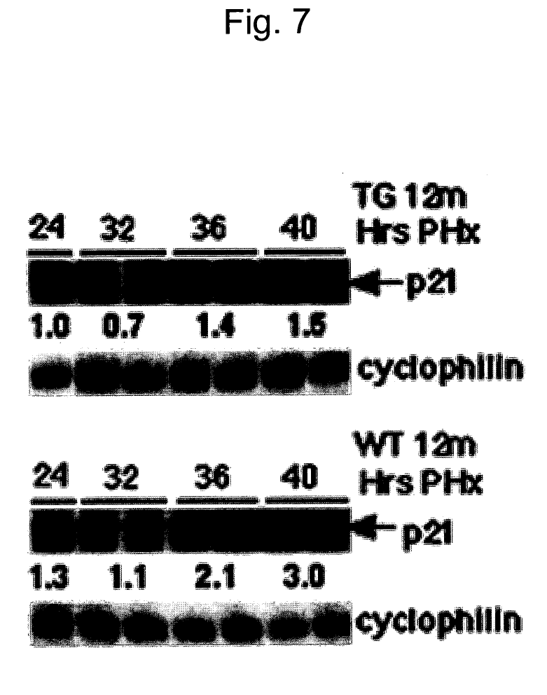

FIG. 7 shows an RNase protection assay of total RNA isolated from regenerating livers of twelve-month-old WT or TG mice using an antisense RNA probe for p21.

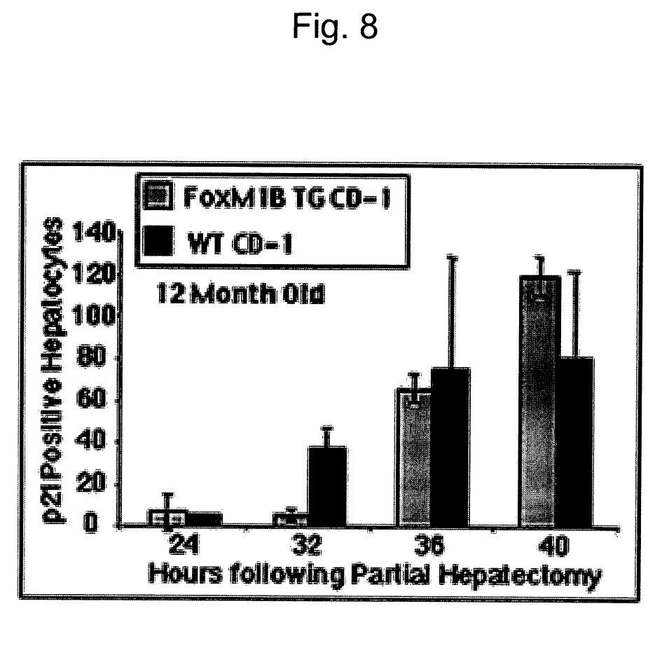

FIG. 8 shows a graph representing the number of p21 positive nuclei per 2500 hepatocytes per regenerating mouse liver, ±the standard deviation (SD).

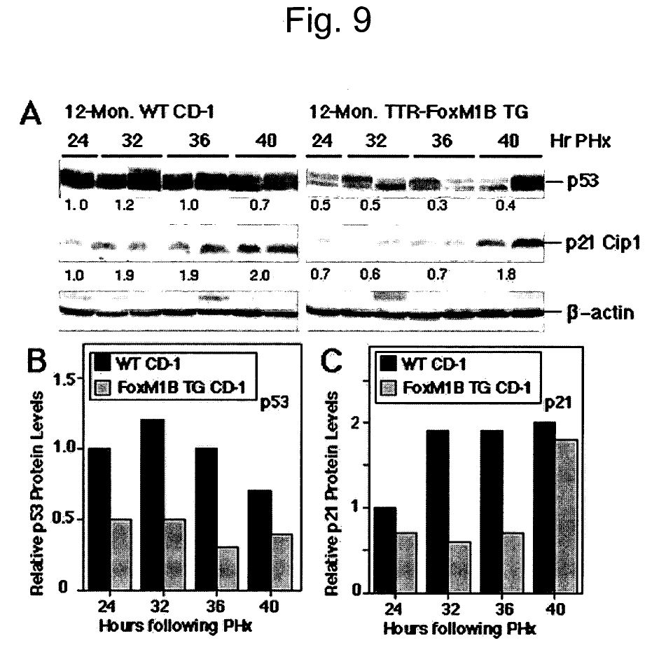

FIG. 9A depicts a Western blot using anti-p53 antibodies showing p53 protein expression in regenerating livers of old-aged TTR-FoxM1B TG mice and old-aged WT mice.

FIGS. 9B-C show graphs depicting relative p53 and p21 protein levels in old aged TTR-FoxM1B transgenic mice compared to levels in old-aged WT mice at various times after PHx.

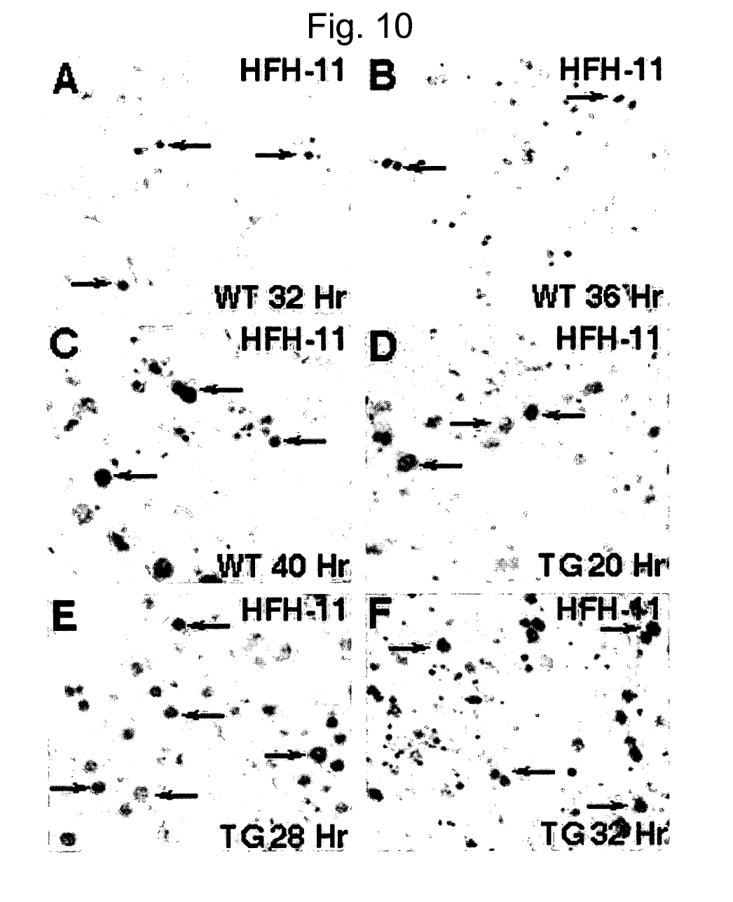

FIGS. 10A through 10F shows immunohistochemical staining of FoxM1B protein with FoxM1B antibody and nuclear expression of FoxM1B protein in CC₁₋₄-treated regenerating liver from WT (FIGS. 10A-C) or TG (FIGS. 10D-F) mice.

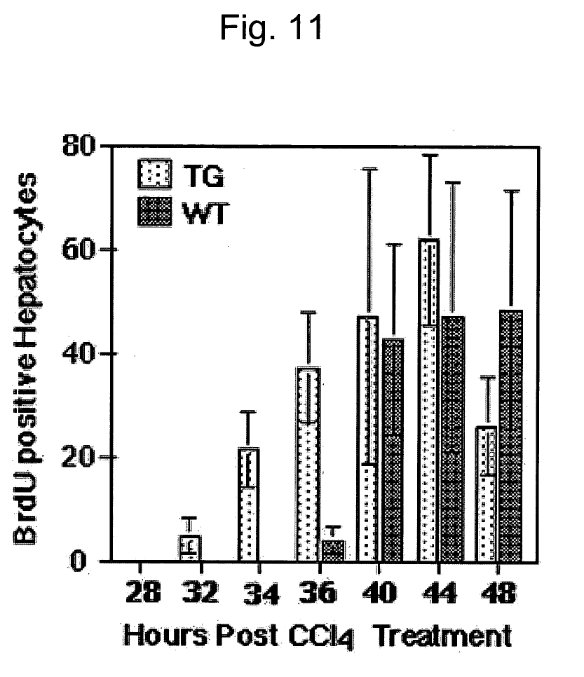

FIG. 11 shows a graph representing BrdU incorporation in hepatocytes at various time points after CCl₄-induced liver damage in WT and TG mice. BrdU positive cells were counted in three viewing fields, each field containing about 250 nuclei.

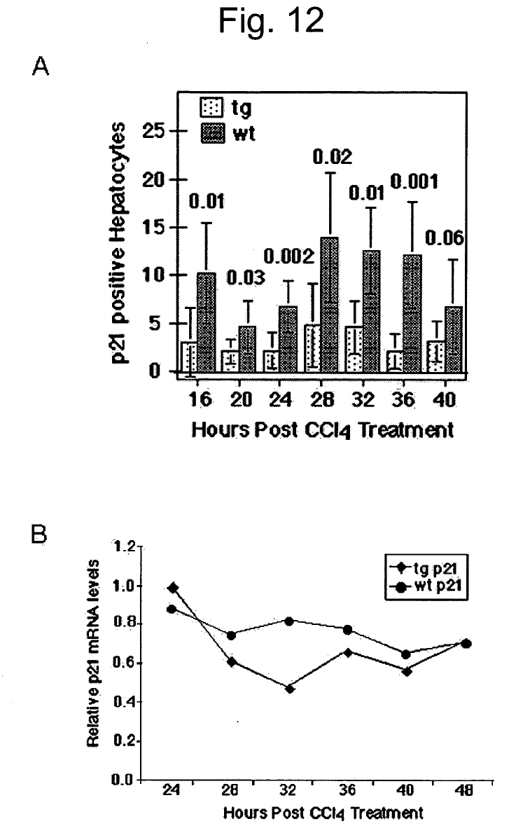

FIG. 12A shows a statistical analysis of p21-staining hepatocytes in WT and TG liver regeneration.

FIG. 12B shows a graph representing levels of p21 mRNA expression in regenerating livers from WT and TG mice, normalized to glyceraldehyde-3-phosphate dehydrogenase (GAPDH) and large ribosomal L32 protein levels.

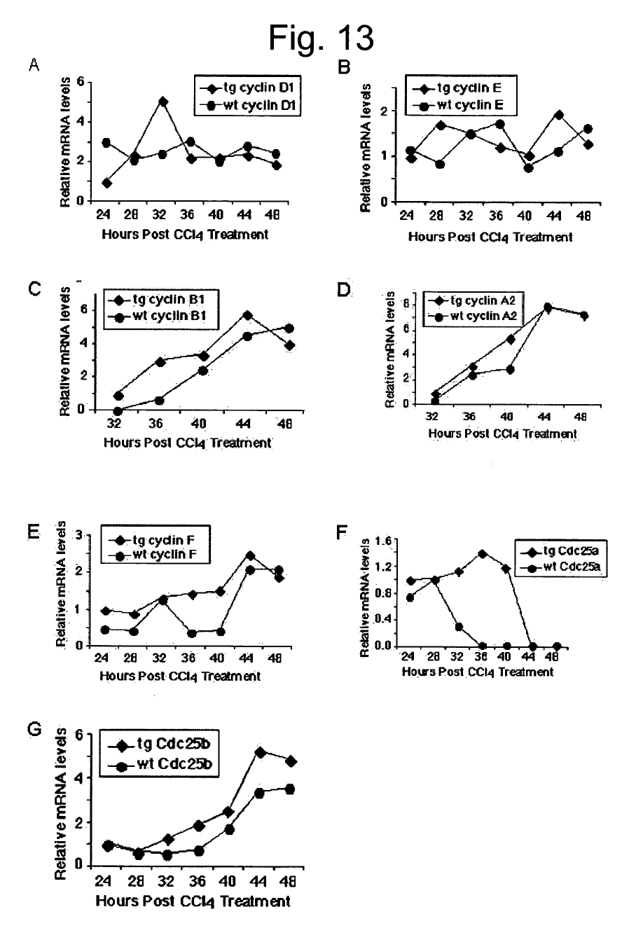

FIG. 13 shows a graph representing Cyclin D1 (A), Cyclin E (B), Cyclin B1 (C), Cyclin A2 (D), Cyclin F (E), Cdc25a (F), and Cdc25b (G) mRNA expression in regenerating WT and TG livers at various times after CCl₄ induced liver damage.

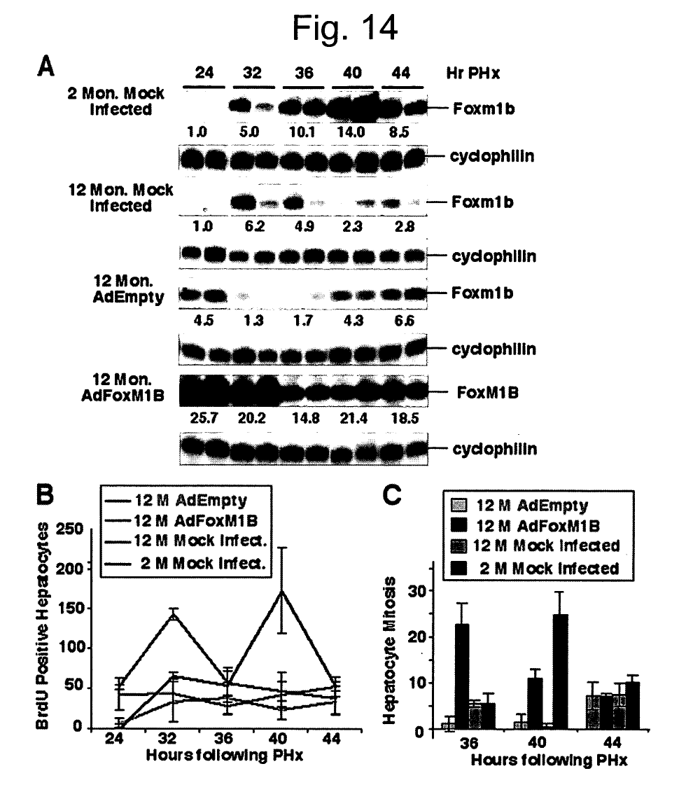

FIG. 14A shows FoxM1B mRNA levels in regenerating livers of old Balb/c mice infected with either AdEmpty (adenovirus control) or AdFoxM1B (adenoviral vector with FoxM1B) two days prior to PHx operation or left uninfected. Expression of FoxM1B mRNA was normalized to cyclophilin levels. Shown below the panel is the fold induction compared to expression levels at the beginning of the experiment (the 0-hour time point).

FIG. 14B shows a graph representing hepatocyte BrdU incorporation during mouse liver regeneration induced by PHx in twelve month-old Balb/c mice infected with either AdFoxM1B or AdEmpty or left uninfected. The mean of the number of BrdU positive nuclei per 1000 hepatocytes and the standard deviation (SD) was calculated for each time point.

FIG. 14C shows a graph representing increased hepatocyte mitosis in regenerating livers of old mice infected with AdFoxM1B between 36 to 44 hours post PHx. Two regenerating livers were used for each time point post PHx. Hepatocyte mitosis is expressed as the mean of the number of mitotic figures found per 1000 hepatocytes±SD.

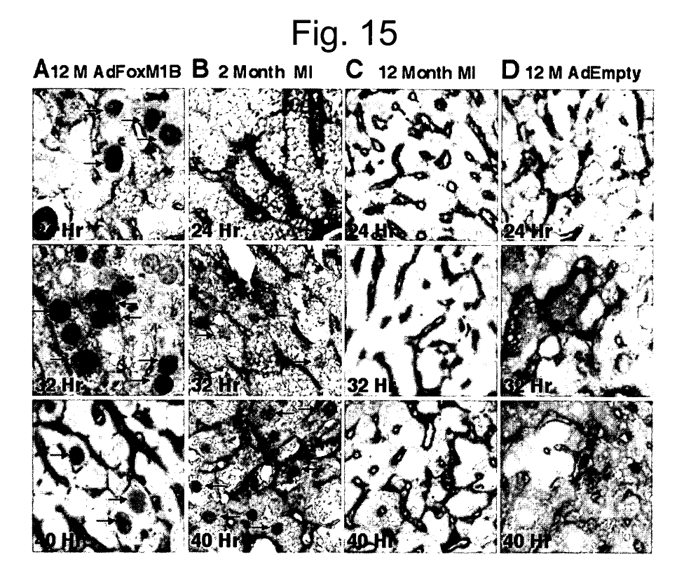

FIG. 15 shows immunohistochemical staining using FoxM1B antibody showing nuclear expression of FoxM1B protein in hepatocytes from regenerating liver of old mice (12 month-old; 12M) infected with AdFoxM1B but not with AdEmpty or mock infected (MI) old aged mice and young (2 month old, 2M) mice.

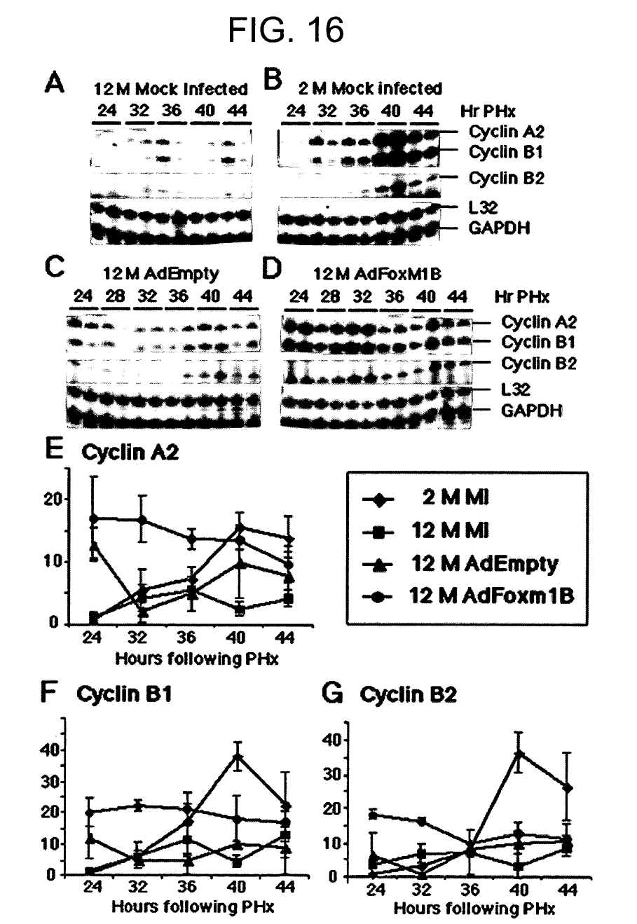

FIGS. 16A through 16D show Northern blot analyses of cyclin gene expression in 12-month and 2-month old mock infected mice and 12 month old mice infected with either AdEmpty or AdFoxM1 B.

FIGS. 16E through 16G are graphs representing stimulated expression of cyclin A2, cyclin B1, and cyclin B2 genes in regenerating liver of old mice infected with AdFoxM1B. Cyclin expression levels were normalized to glyceraldehydes-6-phosphate dehydrogenase (GAPDH) and ribosome large subunit L32 protein mRNA levels.

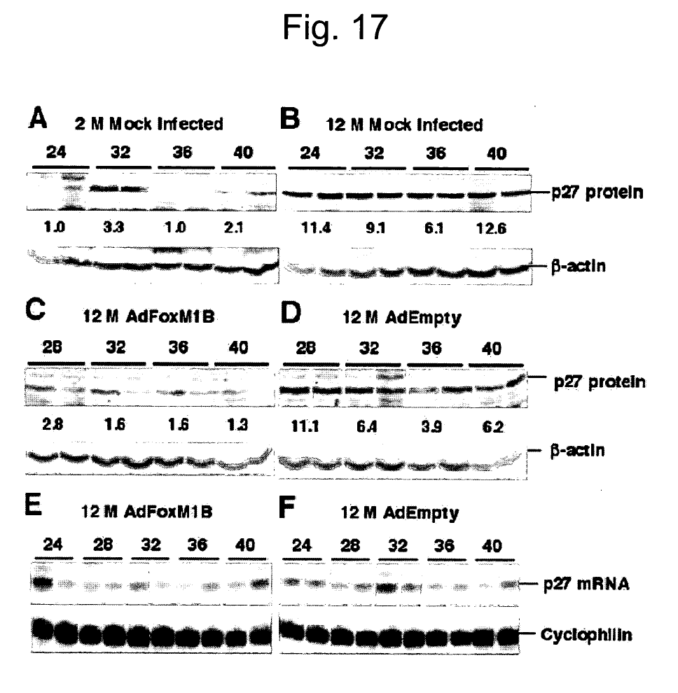

FIGS. 17A-D shows p27 protein expression in regenerating livers from mock-infected (MI) 2-month old (FIG. 17A), MI 12-month old (FIG. 17B), AdFoxM1B infected 12-month old (FIG. 17C), or AdEmpty-infected 12-month old Balb/c mice (FIG. 17D).

FIGS. 17E-F show RNase protection assays demonstrating that p27 mRNA levels are unaffected by AdFoxM1B infection.

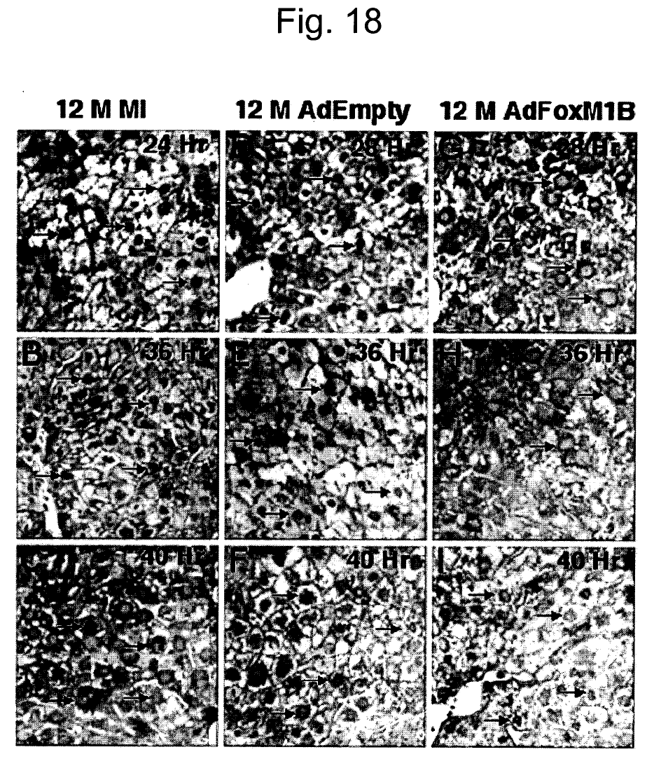

FIG. 18 shows p27 protein localization as detected by immunohistochemical staining with anti-p27 antibodies. Arrows indicate representative p27 staining.

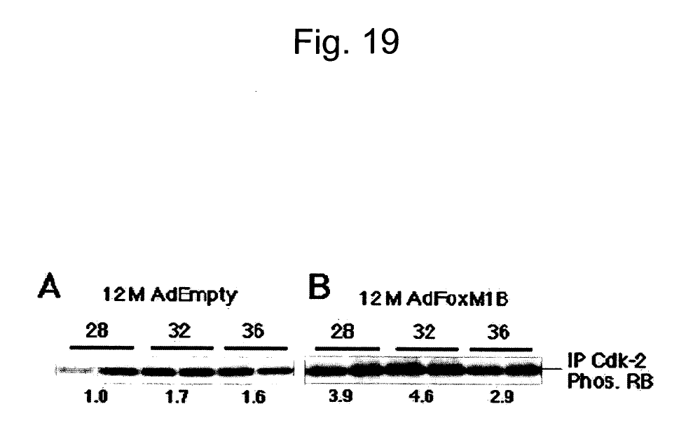

FIG. 19 depicts increased Cdk2 kinase activity in AdFoxM1B-infected 12-month old Balb/c mice compared to AdEmpty infected controls.

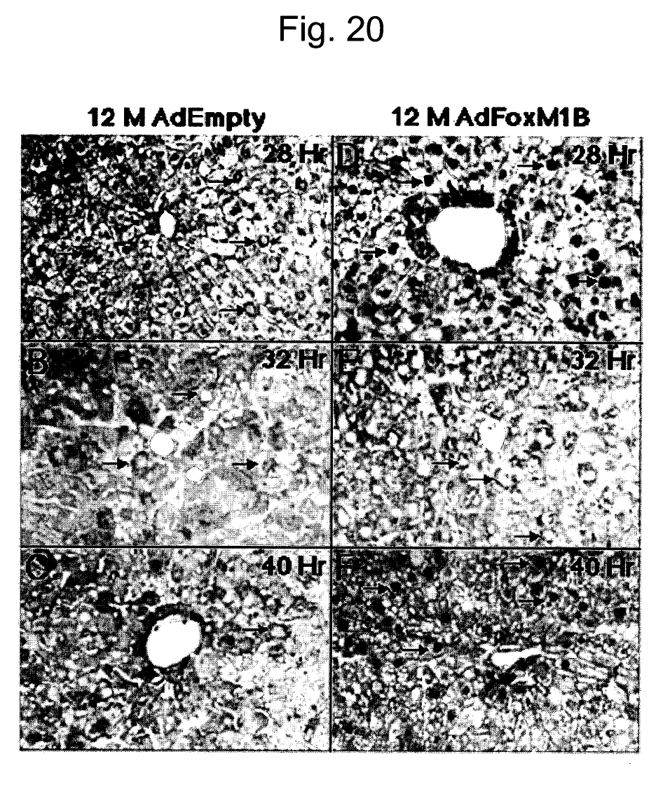

FIG. 20 depicts increased hepatocyte nuclear staining of Cdc25B protein prior to S-phase in liver regeneration of AdFoxM1B-infected 12-month old mice. Arrows indicate representative Cdc25B nuclear staining.

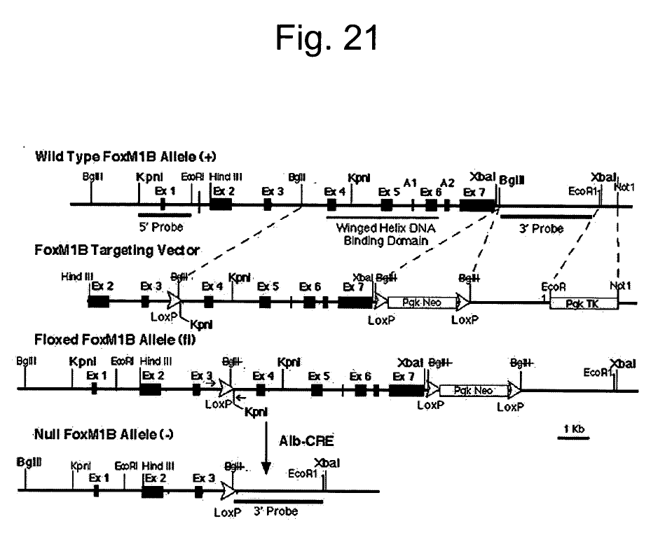

FIG. 21 is a schematic representation of triple-LoxP FoxM1B targeting vector used to generate conditional FoxM1B knockout mice.

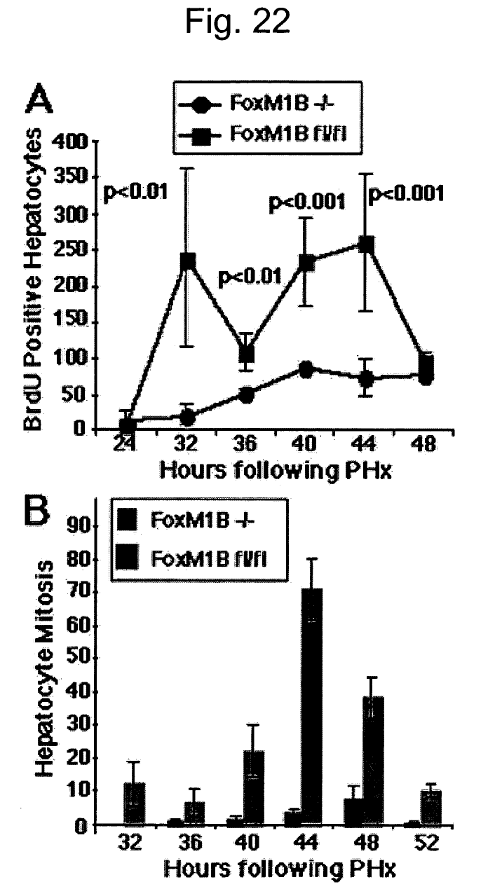

FIG. 22A depicts a graph showing BrdU incorporation in FoxM1B deficient hepatocytes after partial hepatectomy.

FIG. 22B depicts a graph showing hepatocyte mitosis at various times after partial hepatectomy in FoxM1B −/− and FoxM1B fl/fl mice.

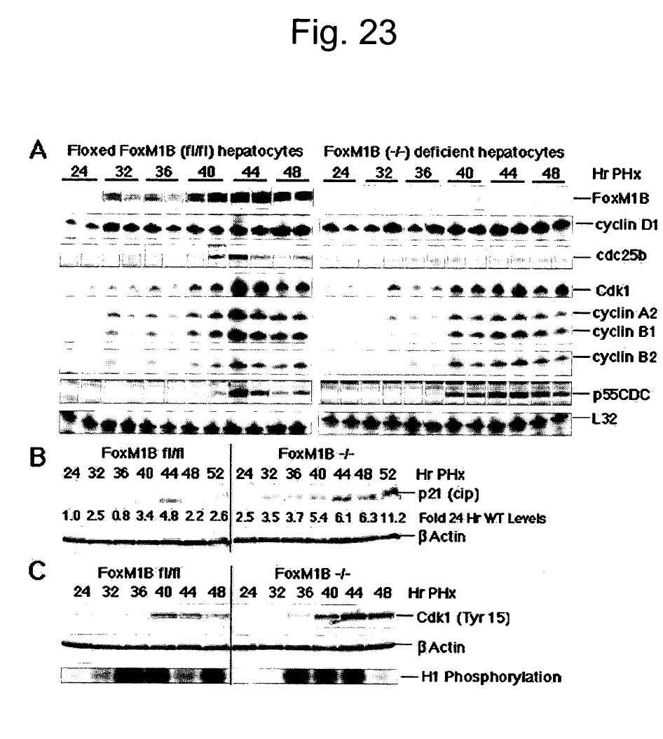

FIG. 23A depicts RNase protection assays performed in duplicate showing expression of cell cycle regulatory genes in regenerating liver of FoxM1B −/− and FoxM1B fl/fl mice.

FIG. 23B depicts a Western blot analysis showing p21 protein levels in regenerating FoxM1B −/− and FoxM1B fl/fl hepatocytes.

FIG. 23C depicts a Western blot analysis with cdk-1 specific phospho-Tyrosine 15 antibodies and kinase assays using H1 protein as a substrate in FoxM1B −/− and FoxM1B fl/fl hepatocytes during liver regeneration.

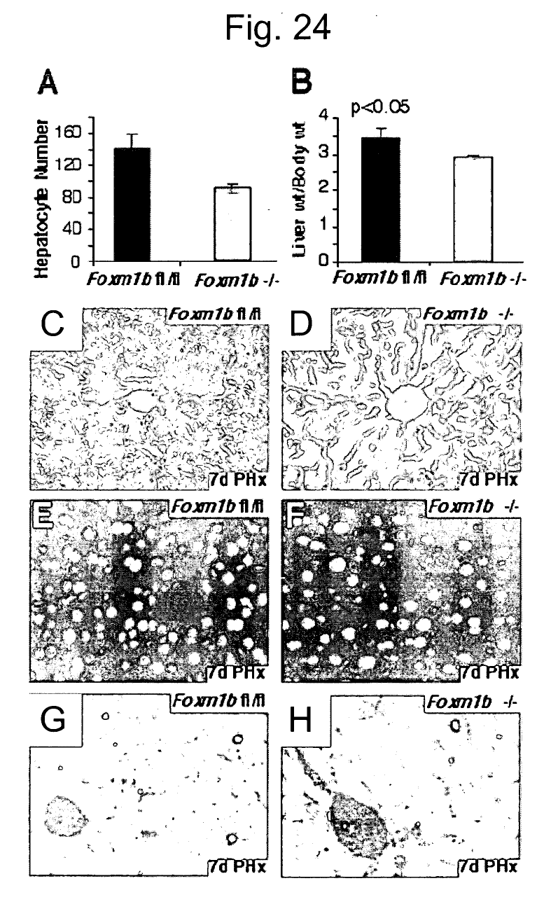

FIG. 24A depicts a graphic representation of a diminished number of hepatocytes in regenerating Alb-Cre FoxM1B −/− liver compared to FoxM1B fl/fl liver. The mean number of hepatocytes was calculated from three regenerating mouse livers±SD. The number of hepatocytes was counted from three liver micrographs under 200× magnification.

FIG. 24B depicts a graphic representation of the liver weight (wt)/body wt at 7 days post PHx showing a compensatory weight increase in the regenerating Alb-Cre FoxM1B −/− liver.

FIGS. 24C-D show hypertrophy of Alb-Cre FoxM1B −/− hepatocytes compared to FoxM1B fl/fl hepatocytes as detected by histological staining with Hematoxylin and Eosin.

FIGS. 24E-F show diminished number of Alb-Cre FoxM1B −/− hepatocyte nuclei compared with FoxM1B fl/fl hepatocyte nuclei at 7 days after PHx as detected by DAPI staining.

FIGS. 24G-H show a TUNEL assay that was conducted on regenerating livers from Alb-Cre FoxM1B −/− and FoxM1B fl/fl mice showing no significant increase in apoptosis in the Alb-Cre FoxM1B −/− liver.

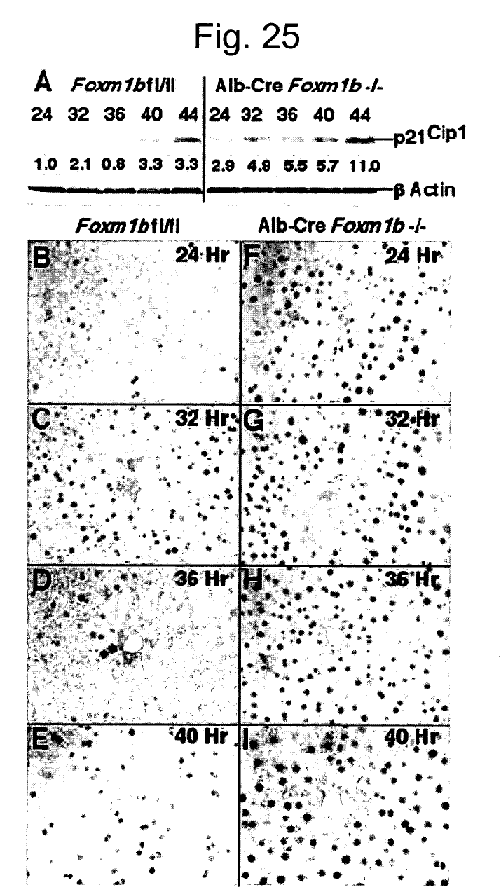

FIG. 25A shows that p21 protein levels detected by Western blot analysis are increased in regenerating Alb-Cre FoxM1B −/− liver.

FIGS. 25B-I shows immunohistochemical staining of regenerating liver sections with anti-p21 antibodies demonstrating nuclear staining of p21 protein in Alb-Cre FoxM1B −/− liver (FIGS. 25F-I) compared to Foxm1b fl/fl littermates (FIGS. 25B-E).

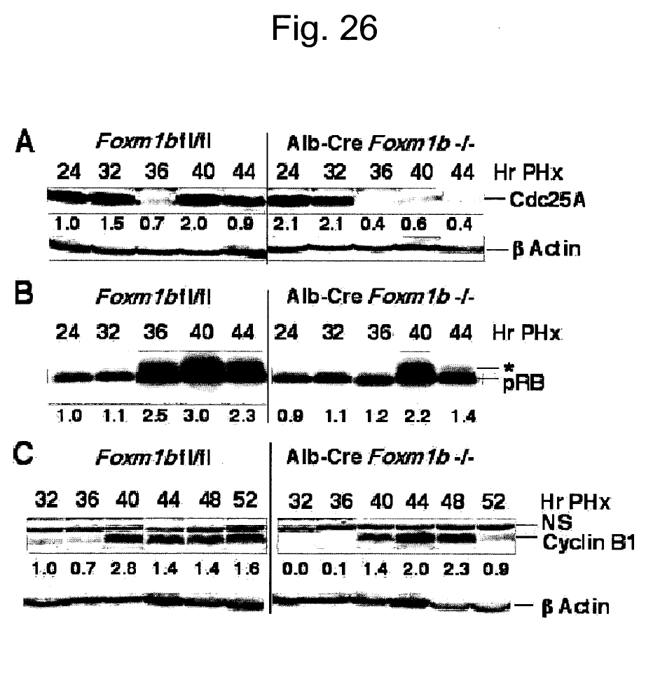

FIGS. 26A-C shows that Cdc25A protein levels and Cdk2 activity are decreased in regenerating Alb-Cre FoxM1B −/− liver as detected by Western blot analysis and kinase assays. In FIG. 26C, a non-specific band that reacted with the anti-Cyclin BI antibodies is labeled NS. In FIGS. 26A and C, anti-β-actin antibodies were used to stain for β-actin protein as a loading control. The numbers below the panels represent the fold increase in expression levels with respect to regenerating FoxM1B fl/fl liver at the 24 or 32 hour time point.

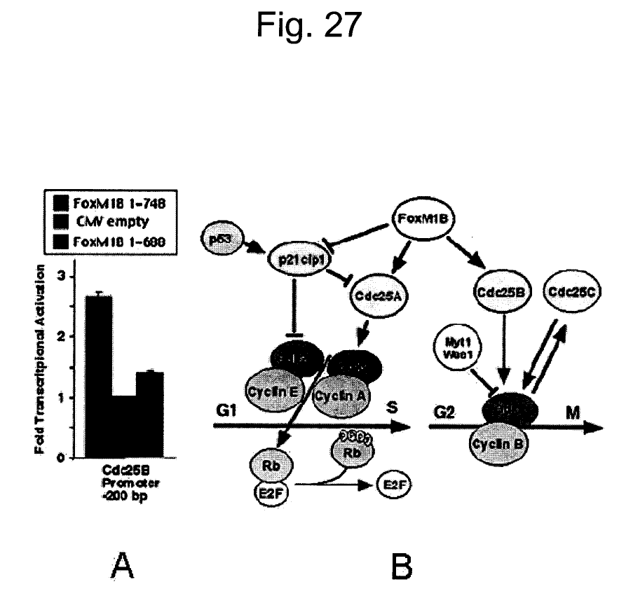

FIG. 27A is a graph depicting the ability of FoxM1B to activate transcription of the Cdc25B promoter in cotransfection assays. The CMV-empty vector control was set at 1.0. Two transfection experiments were performed in duplicate and used to determine the mean fold induction±standard deviation.

FIG. 27B is a diagram depicting FoxM1B regulation of cell cycle genes. The diagonally oriented arrows represent positive regulation and the lines represent negative regulation.

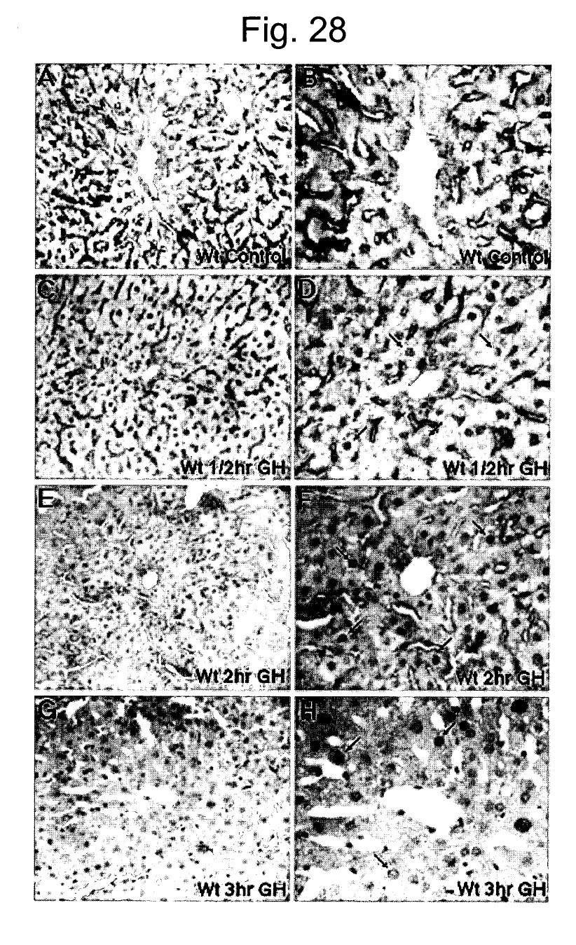

FIG. 28 shows hepatocyte nuclear expression of FoxM1B protein in young CD-1 mice stimulated by growth hormone. Shown are micrographs (200×, left panel and 400×, right panel) of wild-type liver sections displayed FoxM1B nuclear staining (indicated by arrows) between 30 minutes (C-D), 2 hours (E-F) and 3 hours (G-H) following growth hormone administration but not in control mice (A-B).

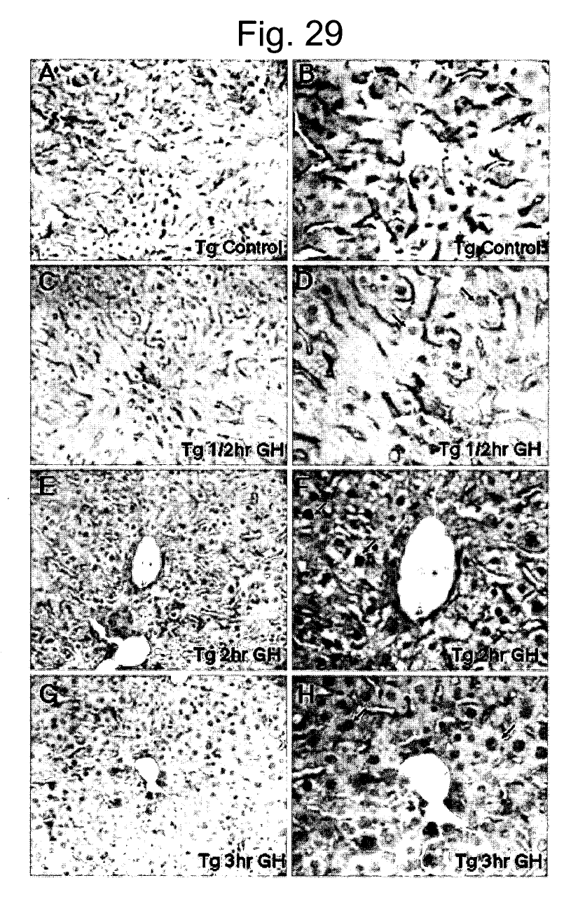

FIG. 29 shows hepatocyte nuclear expression of FoxM1B protein in young TTR-FoxM1B transgenic mice stimulated by growth hormone. Shown are micrographs (200×, left panel and 400×, right panel) of TTR-FoxM1B liver sections displayed FoxM1B nuclear staining (indicated by arrows) between 30 minutes (C-D), 2 hours (E-F) and 3 hours (G-H) following growth hormone administration but not in control transgenic mice (A-B).

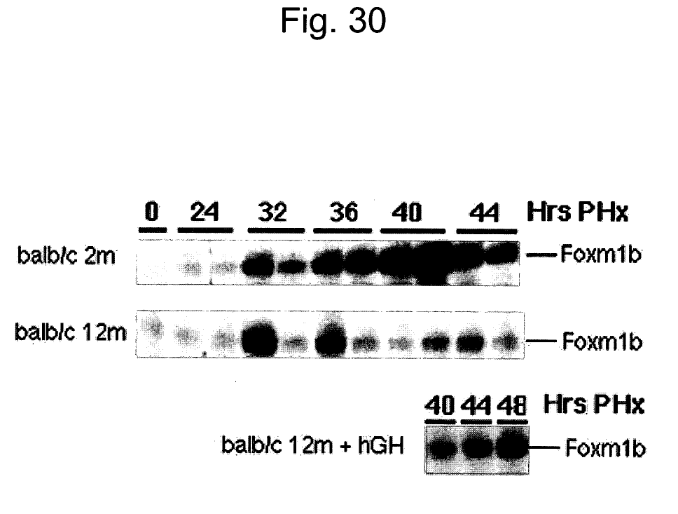

FIG. 30 shows a time course of FoxM1B mRNA levels in regenerating liver of untreated 2-month old (young) and 12-month old Balb/c mice as well as 12-month old Balb/c mice treated with human growth hormone.

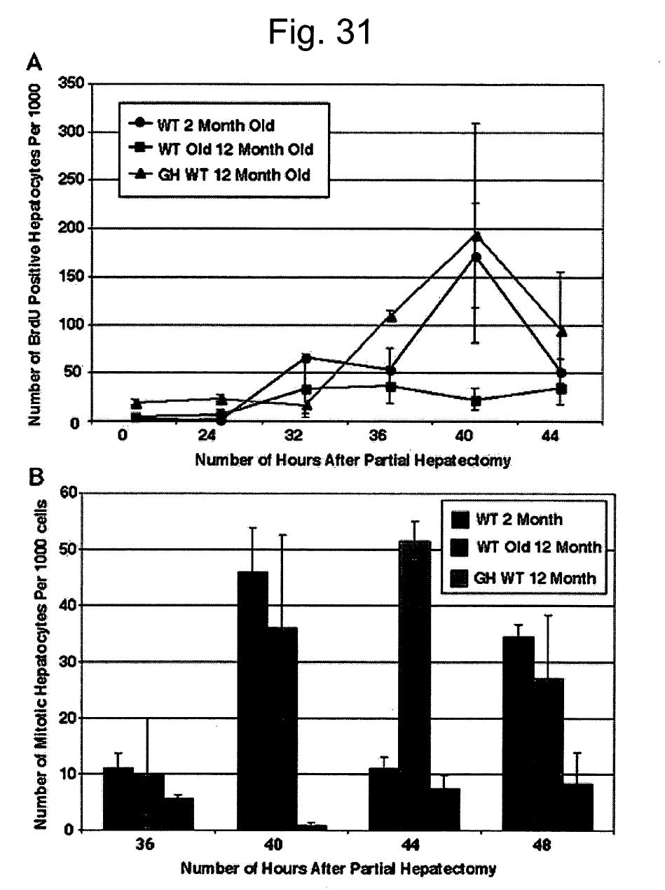

FIG. 31A shows a graph representing number of BrdU positive hepatocytes from regenerating livers in mice treated with growth hormone.

FIG. 31B shows a graph representing number of mitotic hepatocytes from regenerating livers in mice treated with growth hormone.

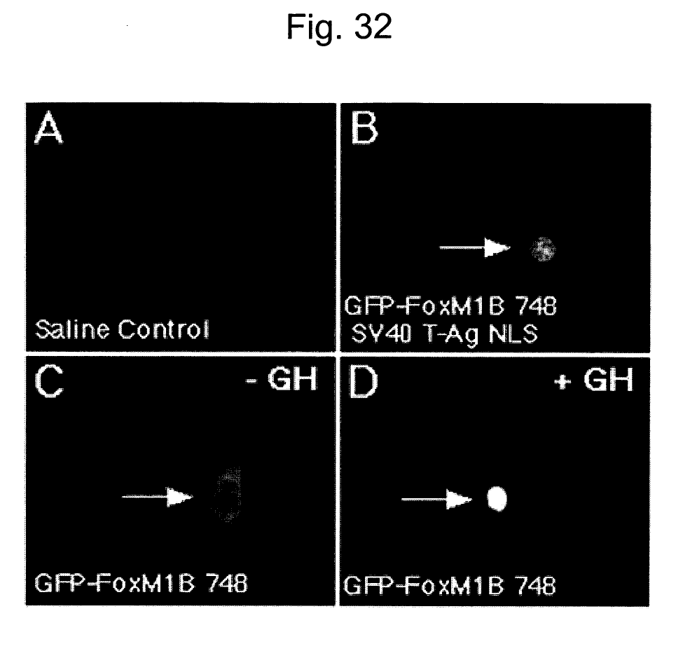

FIGS. 32A-D depicts immunohistochemical staining with FoxM1B antibody showing localization of GFP-FoxM1B-NLS (FIG. 32B) and GFP-FoxM1B in the presence and absence of growth hormone (FIGS. 32C and D). FIG. 32A is a control.

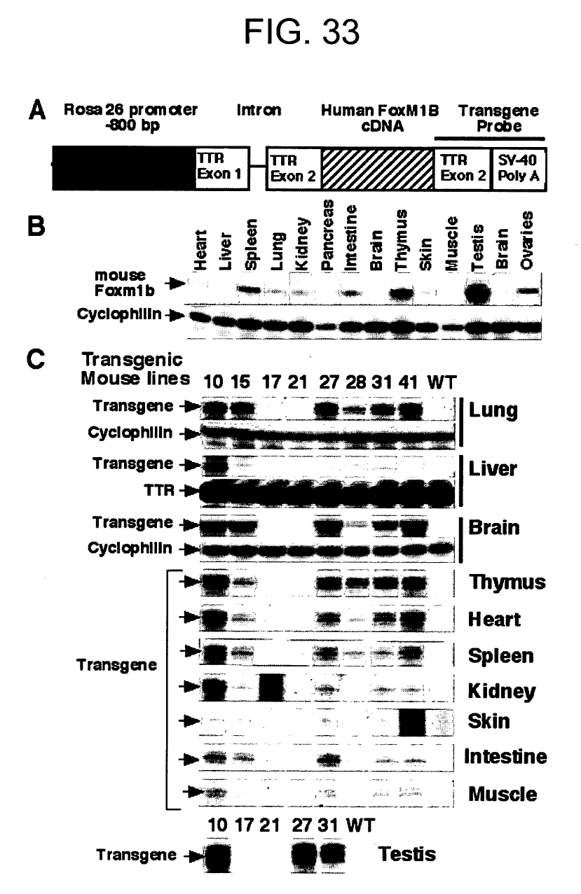

FIG. 33A shows a diagram of the −800 bp mouse Rosa 26 promoter driving expression of the FoxM1B cDNA that is placed within the Transthyretin (TTR) minigene construct. TG mice were created with the Rosa26 promoter region (solid black box) driving expression of the human FoxM1B cDNA (striped box), which was cloned into the TTR second exon that contains the SV40 polyadenylation signal. Also depicted on the diagram is the position of the TTR transgene probe.

FIG. 33B shows RNase protection assays demonstrating that FoxM1B is abundantly expressed in adult thymus and testis with lower levels in spleen, lung, kidney, intestine and ovaries. Total RNA was prepared from different tissues of WT mice and analyzed for mouse FoxM1B and cyclophilin mRNA.

FIG. 33C shows RNase protection assays of transgene expression in Rosa 26 transgenic mice. Total RNA was prepared from different tissues of eight TG mouse lines and analyzed for FoxM1B transgene, mouse TTR and cyclophilin mRNA by RNase protection assays. Transgenic mouse line #10 exhibited high levels of the FoxM1B transgene in lung, liver, brain, thymus, heart, spleen, kidney, intestine, muscle and testis and displayed lower levels in skin.

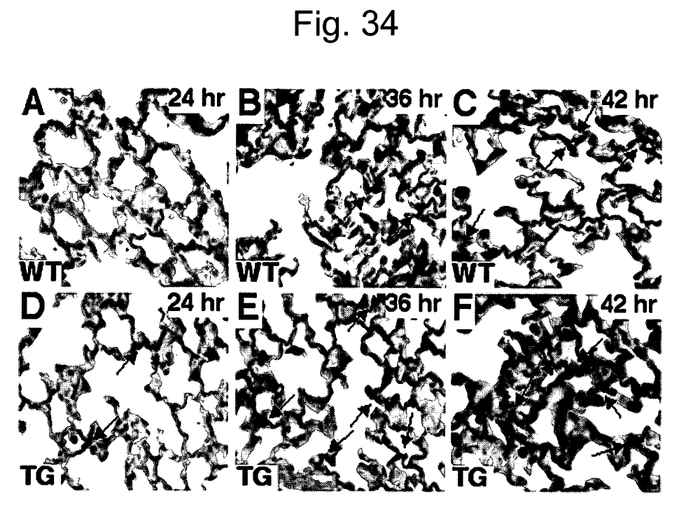

FIGS. 34A-F show immunohistochemical staining of Rosa26-FoxM1B transgenic mice lungs (FIGS. 34A-C) or wild type mice lungs (FIGS. 34D-F) with FoxM1B antibody following BHT injury.

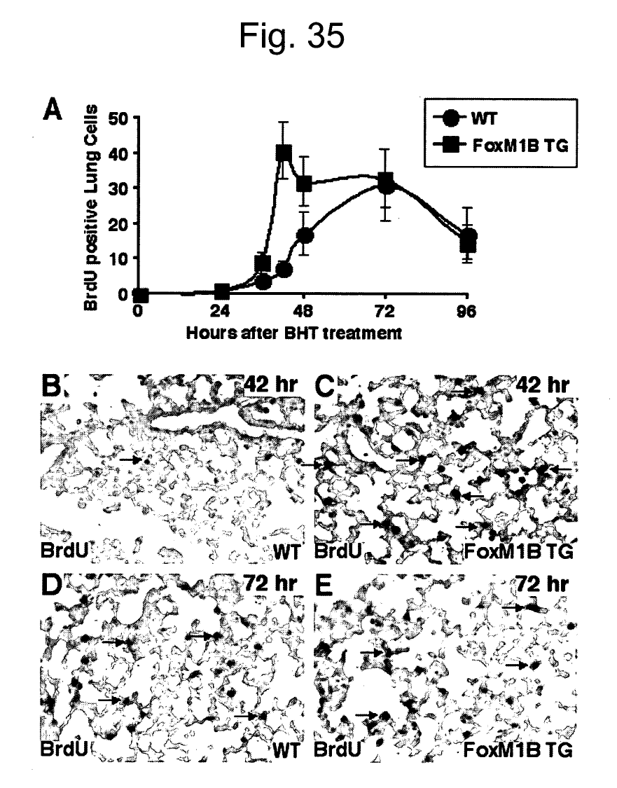

FIGS. 35A-E show that premature expression of FoxM1B accelerates the onset of lung DNA replication after BHT injury.

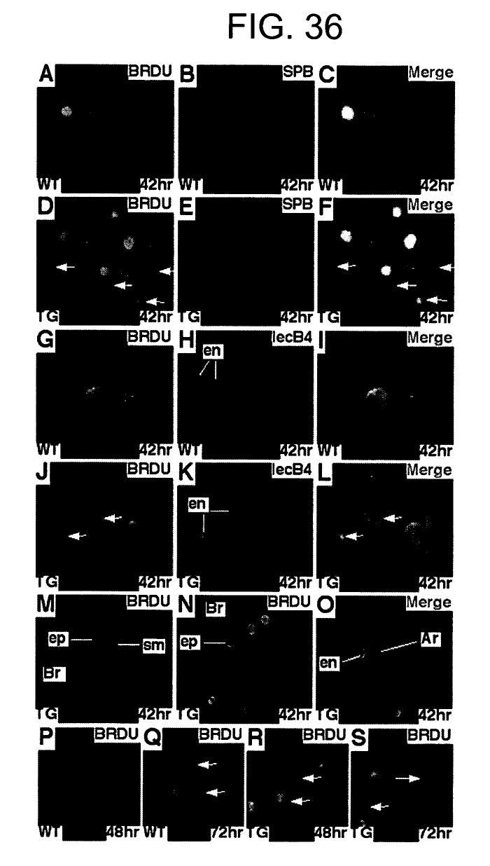

FIG. 36 shows premature expression of FoxM1B causes earlier DNA replication of pulmonary epithelial, endothelial and smooth muscle cells following BHT injury. Paraffin sections were prepared from lungs of Rosa26-FoxM1B transgenic (TG) and wild type (WT) mice following BHT lung injury. DNA replication was detected with BrdU monoclonal antibody and an anti-mouse antibody conjugated to TRITC (FIGS. 36A, D, G, J and M). Type II epithelial cells were stained with SPB antibody detected by anti-rabbit antibody conjugated to FITC (FIGS. 36B, E) or endothelial cells were visualized using FITC-conjugated isolectin B4 from Griffonia Simplicifolia (lecB4; FIGS. 36H and K). (FIGS. 36A-F) Regenerating TG lungs exhibit a greater number of BrdU positive epithelial cells (dark arrows) than regenerating WT lungs. Only regenerating TG lungs exhibited BrdU positive small nuclei, which are likely endothelial cells (white arrows) at 42 hours following BHT injury. BrdU staining is shown for WT (FIG. 36A) and TG (FIG. 36D) lungs, SPB staining for WT (FIG. 36B) and TG (FIG. 36E) lungs and merging of this staining (FIGS. 36C and F). (FIGS. 36G-L) Regenerating TG lungs displayed premature proliferation of endothelial capillary cells (lecB4; white arrows). BrdU staining is shown for WT (FIG. 36G) and TG (FIG. 36J) lungs, lecB4 endothelial cell staining for WT (FIG. 36H) and TG (FIG. 36K) lungs and merging of this staining (FIGS. 36I and L). FIGS. 36M-O show BrdU staining of TG lungs depicts earlier proliferation in peribronchiolar smooth muscle cells (FIG. 36M), bronchial epithelial cells (FIG. 36N) and arteriolar endothelial cells (FIG. 36O) at 42 hours following BHT injury. (FIGS. 36P-S) TG lungs display BrdU positive small nuclei at 48 and 72 hours following BHT injury (R-S, white arrows) whereas WT lungs show this only at 72 hours (FIG. 36Q). Abbreviations are as follows: en, endothelial cells; ep, epithelial cells; sm, smooth muscle cells; Br, bronchiole; and Ar, artery. Magnification: A-F and M-S is 400×; G-L is 630×.

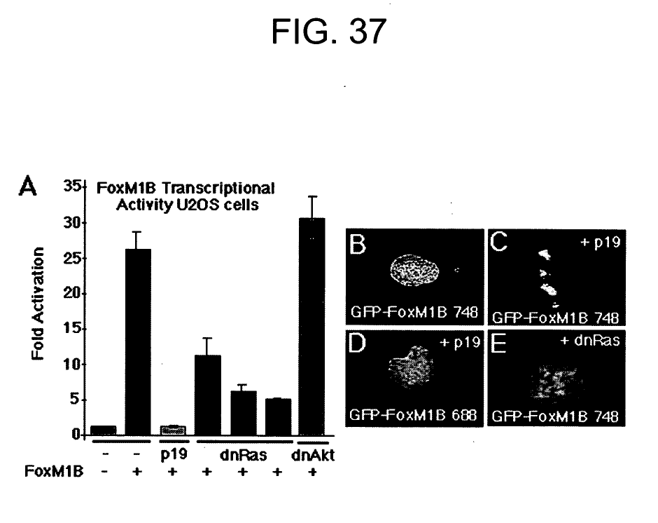

FIG. 37A shows a graph representing the fold FoxM1B transcriptional induction with cotransfection of p19, CMV-dominant negative Ras (dnRas), or CMV-dominant negative AKT (dnAKT) {CMV-empty vector control (−) set at 1.0}. Three distinct transfections were used to determine mean fold induction ±SD.

FIGS. 37B-E show that cotransfection of p19 mediates targeting of the GFP-FoxM1B fusion protein to the nucleolus as detected by immunofluorescence assays of expression constructs comprising CMV promoter driven green fluorescent protein (GFP) fused to either full length FoxM1B protein (amino acid 1 to 748; B, C, and E) or the transcriptionally inactive C-terminal deletion FoxM1B protein (amino acids 1 to 688; D).

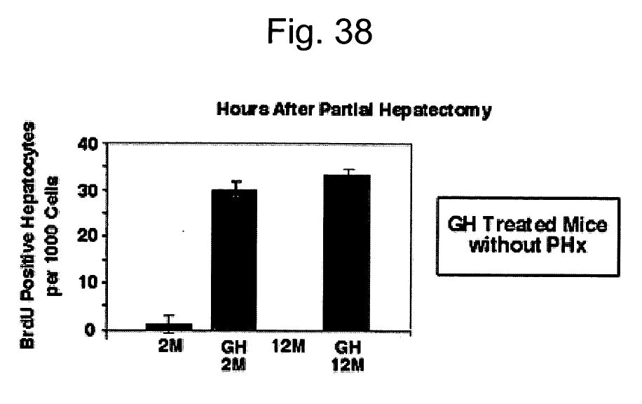

FIG. 38 shows a graph depicting hepatocyte proliferation in both young and old-aged mice treated with growth hormone but not subjected to PHx. The mean of the number of BrdU-positive nuclei per 1,000 hepatocytes±SD was calculated at 41 hours following the first growth hormone injection from three distinct mouse livers.

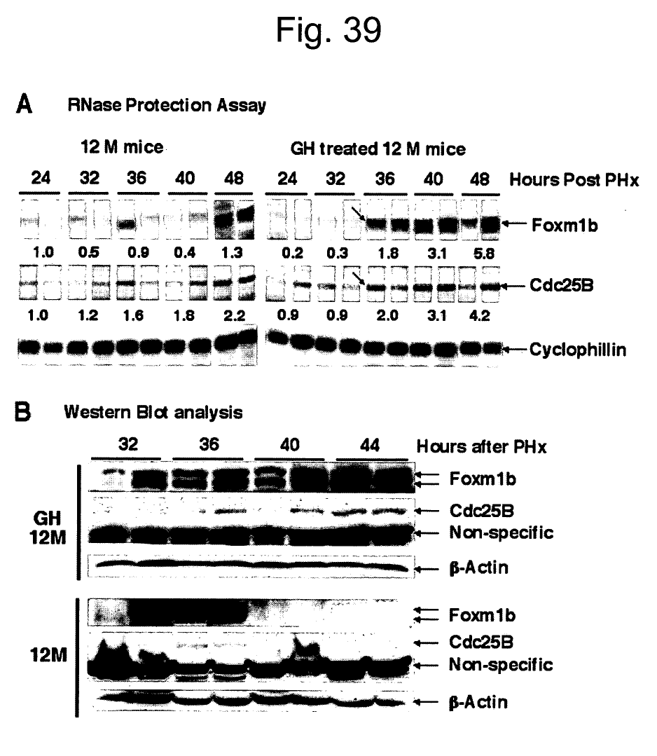

FIG. 39A shows an RNase protection assay for FoxM1B and Cdc25B mRNA expression in regenerating liver of old-aged (12 month-old) untreated or growth hormone (GH) treated Balb-c mice. Shown below each panel is the average fold induction of mRNA levels compared with that of untreated 12 month-old mice at 24 hours after PHx.

FIG. 39B shows Western blots depicting Foxm1b and Cdc25B protein expression in regenerating liver of GH treated 12 month-old Balb-c mice. β-Actin was used as a loading control.

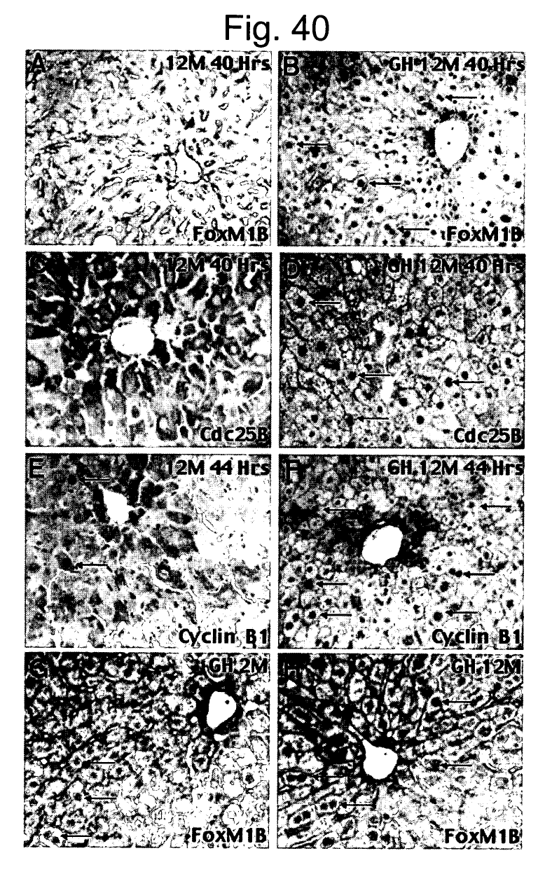

FIGS. 40A-H shows immunohistochemical staining of paraffin-embedded regenerating liver sections with FoxM1B (FIGS. 40A, B, G, and H), Cdc25B (FIGS. 40C and D) or Cyclin B1 (FIGS. 40E and F) antibodies. Panels show regenerating liver sections from either untreated 12 month-old mice (FIGS. 40A, C and E) or human growth hormone (GH) treated 12 month-old mice (FIGS. 40B, D and F) at the indicated times after PHx. FIGS. 40G and H show FoxM1B expression in 2 month or 12 month-old livers not subjected to PHx. Arrows indicate representative nuclear staining.

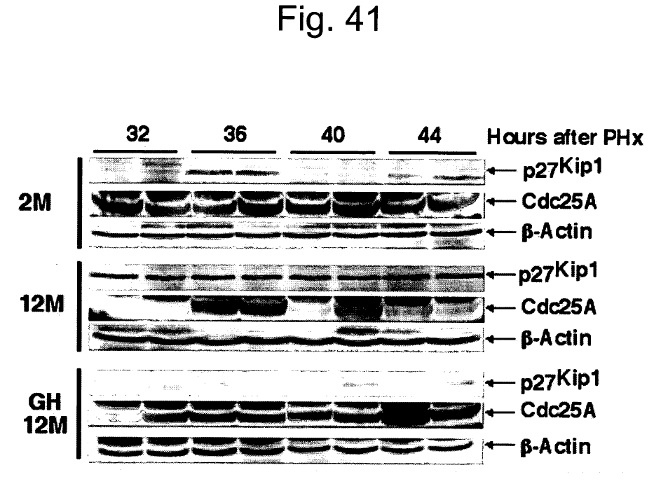

FIG. 41 shows Western blot analysis depicting p27^{kip1} (p27) and Cdc25A protein expression in regenerating liver of untreated two month-old (2M), untreated 12 month-old (12M) and growth hormone treated 12 month-old (GH 12M) Balb/c mice. β-Actin was used as a loading control.

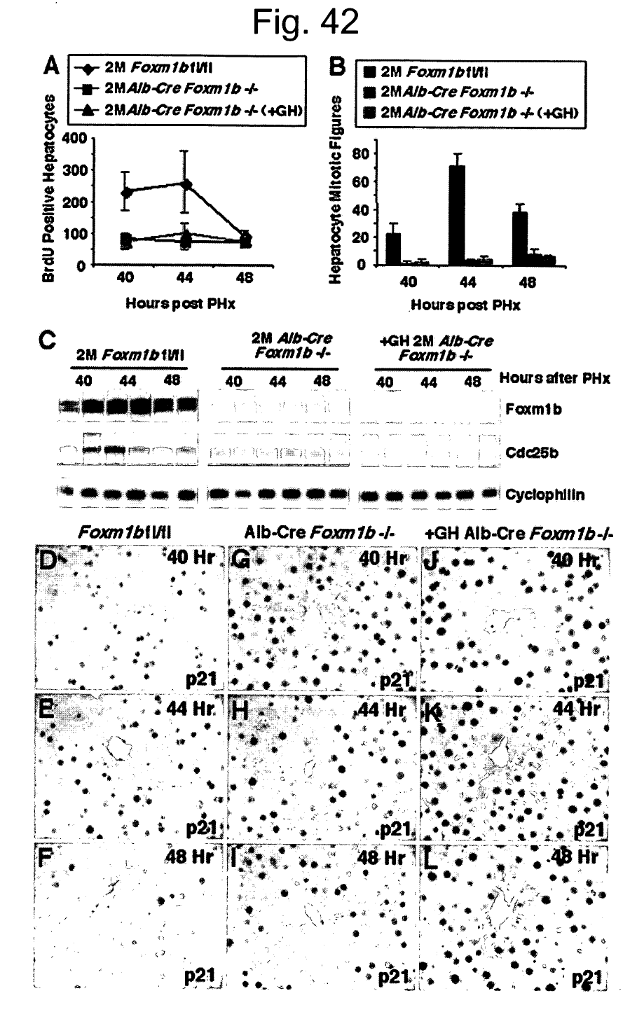

FIG. 42A is a graphic representation of hepatocyte BrdU incorporation during liver regeneration in Foxm1b fl/fl and untreated or growth hormone (GH) treated Alb-Cre Foxm1b −/− mice.

FIG. 42B is a graphic representation of hepatocyte mitosis during liver regeneration in untreated or growth hormone treated Alb-Cre Foxm1b −/− mice and Foxm1b fl/fl littermates. Three mice are represented for each time point in all calculations.

FIG. 42C shows RNase protection assays depicting FoxM1B and Cdc25B mRNA expression in regenerating liver of GH treated Alb-Cre Foxm1b −/− mice.

FIGS. 42D-L shows immunohistochemical staining of regenerating liver sections with p21^{Cip1} antibody from untreated or GH treated Alb-Cre Foxm1b −/− mice compared to Foxm1b fl/fl control mice.

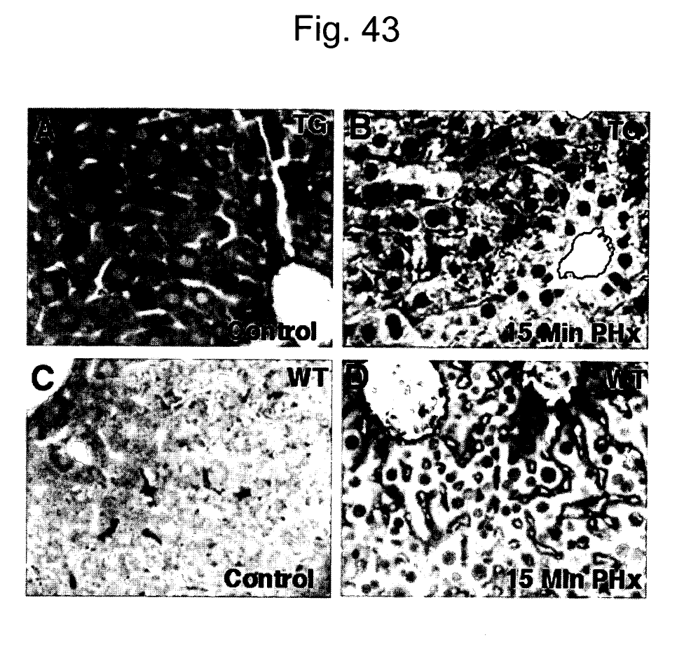

FIG. 43 shows immunohistochemical staining demonstrating rapid nuclear localization of FoxM1B transgene protein in regenerating transgenic hepatocytes immediately following partial hepatectomy. Eight-week-old wild type (WT) and TTR-FoxM1B transgenic (TG) CD1 mice were subjected to two-thirds partial hepatectomy (PHx) and regenerating livers were harvested 15 minutes after beginning the PHx surgery (B and D) or without surgery (control, A and C). Shown is regenerating or hepatocyte staining of FoxM1B protein in liver sections from either TG (A-B) or WT (C-D) mice. Magnification is 400×.

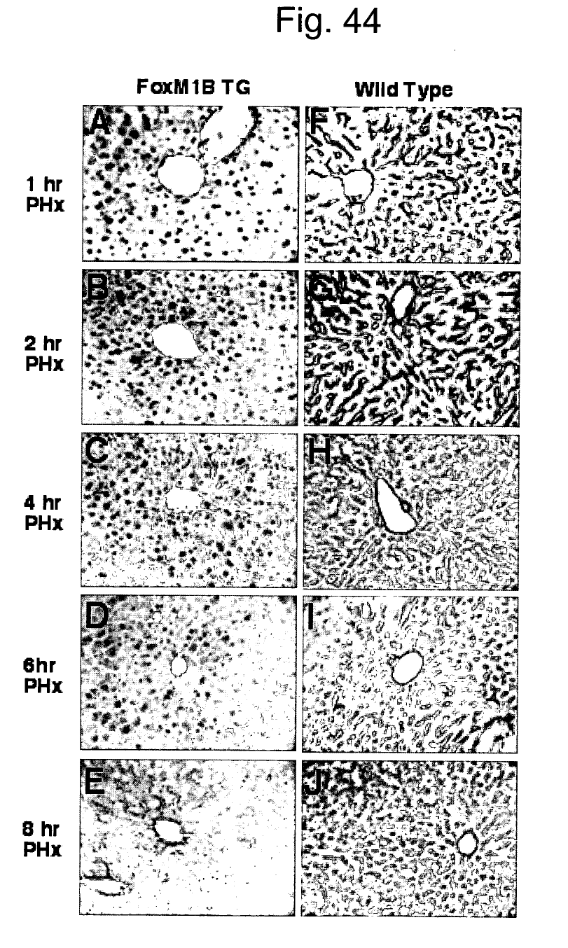

FIGS. 44A-J shows immunohistochemical staining demonstrating nuclear translocation of the FoxM1B transgene protein within the first six hours following partial hepatectomy. Eight-week-old WT (FIGS. 44F-J) and TG (FIGS. 44A-E) CD1 mice were subjected to two-thirds partial hepatectomy (PHx) and regenerating livers were harvested at either 1, 2, 4, 6 or 8 hours (hrs) following PHx and used for immunohistochemical staining with affinity-purified FoxM1B specific antibody. Magnification is 200×.

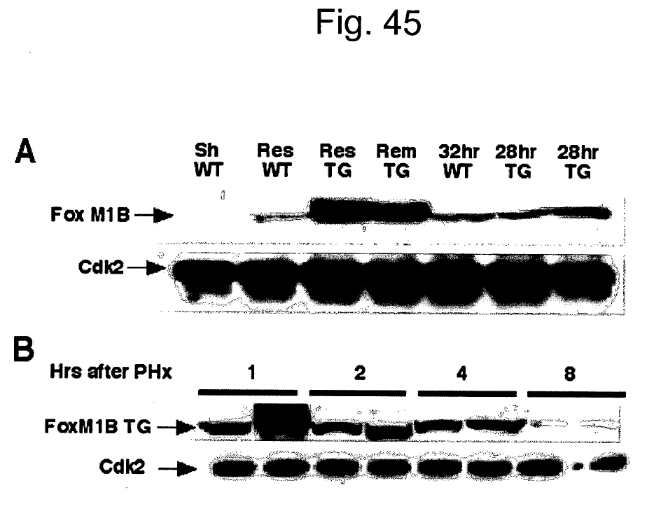

FIG. 45A shows a FoxM1B Western Blot analysis of regenerating liver nuclear extracts from WT and transgenic mice immediately after PHx. Nuclear extracts were prepared from regenerating liver tissue 15 minutes after PHx, sham operated (Sh) or regenerating wild type (WT) and transgenic (TG) liver at the indicated times following PHx as described previously. Lanes marked Res (Resected) were nuclear extracts prepared from the liver tissue removed during partial hepatectomy (PHx). Lanes marked Rem (Remnant) were nuclear extracts were prepared immediately after PHx from the remaining liver tissue. Nuclear protein extracts from regenerating WT liver at 32 hours following PHx and duplicate regenerating TG liver at 28 hrs after PHx were included for comparison. Two hundred μg of liver nuclear extract was analyzed by Western blot analysis with polyclonal antibodies against either FoxM1B or Cdk2 (loading control).

FIG. 45B shows a Western blot analysis of nuclear extracts prepared from regenerating TG liver prepared from two distinct mice (1, 2, 4 or 8 hours following PHx) with FoxM1B antibody.

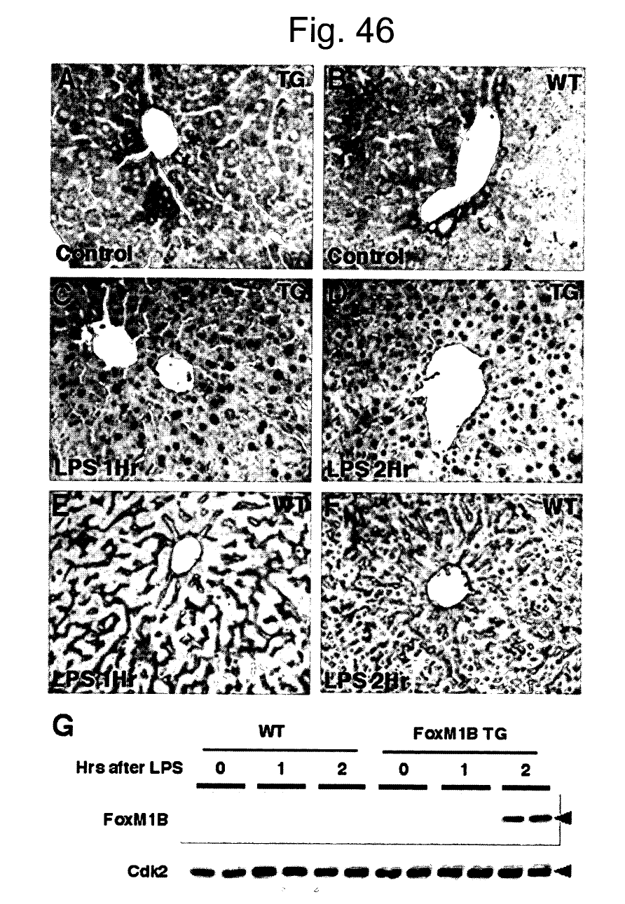

FIGS. 46A-G shows increased hepatocyte nuclear staining of the FoxM1B transgene protein during the hepatic acute phase response. Eight-week-old WT (FIGS. 46B and E-F) and TG (FIGS. 46A and C-D) CD1 mice were subjected to lipopolysaccharide (LPS) injection and livers were harvested at either 0 (control; FIGS. 46A-B), 1 hour (FIGS. 46C and E) or 2 hours (FIGS. 46D and F) following LPS treatment and used for immunohistochemical staining with affinity-purified FoxM1B antibody. (FIG. 46G) Western blot analysis of nuclear extracts prepared from WT and TG liver following LPS treatment with FoxM1B antibody. Nuclear extracts were prepared from WT and TG liver isolated at 0, 1, or 2 hrs following LPS treatment and used for Western blot analysis with either FoxM1B or Cdk2 (loading control) specific antibodies. The numbers above the panels refer to the hours following LPS treatment (Hrs after LPS). Magnification is 200×.

### DETAILED DESCRIPTION OF THE PREFERRED EMBODIMENTS

Conventional techniques were used for recombinant DNA production, oligonucleotide synthesis, and tissue culture and cell transformation (e.g., electroporation, lipofection) procedures. Enzymatic reactions and purification techniques were performed according to manufacturers' specifications or as commonly accomplished in the art or as described herein. The techniques and procedures were generally performed according to methods well known in the art and as described in various general and more specific references that are cited and discussed throughout the present specification. See e.g., Sambrook et al., 2001, MOLECULAR CLONING: A LABORATORY MANUAL, 3d ed., Cold Spring Harbor Laboratory Press, Cold Spring Harbor, N.Y., which is incorporated herein by reference for any purpose. Unless specific definitions are provided, the nomenclature utilized in connection with, and the laboratory procedures and techniques of, molecular biology, genetic engineering, analytical chemistry, synthetic organic chemistry, and medicinal and pharmaceutical chemistry described herein are those well known and commonly used in the art. Standard techniques can be used for chemical syntheses, chemical analyses, pharmaceutical preparation, formulation, and delivery, and treatment of patients.

Unless otherwise required by context, singular terms shall include pluralities and plural terms shall include the singular.

Definitions

As utilized in accordance with the present disclosure, the following terms, unless otherwise indicated, shall be understood to have the following meanings:

The term “isolated protein” referred to herein means a protein encoded by genomic DNA, cDNA, recombinant DNA, recombinant RNA, or synthetic origin or some combination thereof, which (1) is free of at least some proteins with which it would normally be found, (2) is essentially free of other proteins from the same source, e.g., from the same cell or species, (3) is expressed by a cell from a different species, (4) has been separated from at least about 50 percent of polynucleotides, lipids, carbohydrates, or other materials with which it is naturally found when isolated from the source cell, (5) is not linked (by covalent or noncovalent interaction) to all or a portion of a polypeptide to which the “isolated protein” is linked in nature, (6) is operatively linked (by covalent or noncovalent interaction) to a polypeptide with which it is not linked in nature, or (7) does not occur in nature. Preferably, the isolated protein is substantially free from other contaminating proteins or polypeptides or other contaminants that are found in its natural environment that would interfere with its therapeutic, diagnostic, prophylactic or research use.

The terms “polypeptide” or “protein” is used herein to refer to native proteins, that is, proteins produced by naturally-occurring and specifically non-recombinant cells, or genetically-engineered or recombinant cells, and comprise molecules having the amino acid sequence of the native protein, or sequences that have deletions, additions, and/or substitutions of one or more amino acids of the native sequence. The terms “polypeptide” and “protein” specifically encompass FoxM1B, or species thereof that have deletions, additions, and/or substitutions of one or more amino acids of FoxMIB having at least one functional property of the FoxM1B protein.

The term “naturally-occurring” as used herein refers to an object that can be found in nature, for example, a polypeptide or polynucleotide sequence that is present in an organism (including a virus) that can be isolated from a source in nature and which has not been intentionally modified by man. The term “naturally occurring” or “native” when used in connection with biological materials such as nucleic acid molecules, polypeptides, host cells, and the like, refers to materials which are found in nature and are not manipulated by man. Similarly, “recombinant,” “non-naturally occurring” or “non-native” as used herein refers to a material that is not found in nature or that has been structurally modified or synthesized by man.

As used herein, the twenty conventional amino acids and their abbreviations follow conventional usage. See IMMUNOLOGY—A SYNTHESIS, 2nd Edition, (E. S. Golub and D. R. Gren, Eds.), 1991, Sinauer Associates, Sunderland, Mass., which is incorporated herein by reference for any purpose. According to certain embodiments, single or multiple amino acid substitutions (in certain embodiments, conservative amino acid substitutions) may be made in the naturally-occurring sequence (in certain embodiments, in the portion of the polypeptide outside the domain(s) forming intermolecular contacts or comprising functional domains). In certain embodiments, a conservative amino acid substitution does not substantially change the structural characteristics of the parent sequence (e.g., a replacement amino acid should not disrupt secondary structure that characterizes the parent or native protein, such as a helix). Examples of art-recognized polypeptide secondary and tertiary structures are described in PROTEINS, STRUCTURES AND MOLECULAR PRINCIPLES (Creighton, Ed.), 1984, W. H. New York: Freeman and Company; INTRODUCTION TO PROTEIN STRUCTURE (Branden and Tooze, eds.), 1991, New York: Garland Publishing; and Thornton et at., 1991, Nature 354: 105, which are each incorporated herein by reference.

Naturally occurring residues may be divided into classes based on common side chain properties: 1) hydrophobic: norleucine, Met, Ala, Val, Leu, Ile; 2) neutral hydrophilic: Cys, Ser, Thr, Asn, Gln; 3) acidic: Asp, Glu; 4) basic: His, Lys, Arg; 5) residues that influence chain orientation: Gly, Pro; and 6) aromatic: Trp, Tyr, Phe.

Conservative amino acid substitutions may encompass non-naturally occurring amino acid residues, which are typically incorporated by chemical peptide synthesis rather than by synthesis in biological systems. These include peptidomimetics and other reversed or inverted forms of amino acid moieties.

In contrast, non-conservative substitutions may involve the exchange of a member of one of these classes for a member from another class. Such substituted residues may be introduced into regions of a protein or polypeptide that are homologous with non-human orthologs thereof, or into the non-homologous regions of the molecule.

In making such changes, according to certain embodiments, the hydropathic index of amino acids may be considered. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5) (Kyte et al., 1982, J. Mol. Biol. 157:105-131).

The importance of the hydropathic amino acid index in conferring interactive biological function on a protein is understood in the art (see, for example, Kyte et al., 1982, ibid.). It is known that certain amino acids may be substituted for other amino acids having a similar hydropathic index or score and still retain a similar biological activity. In making changes based upon the hydropathic index, in certain embodiments, the substitution of amino acids whose hydropathic indices are within ±2 is included. In certain embodiments, those that are within ±1 are included, and in certain embodiments, those within ±0.5 are included.

It is also understood in the art that the substitution of like amino acids can be made effectively on the basis of hydrophilicity, particularly where the biologically functional protein or peptide thereby created is intended for use in immunological embodiments, as in the present case. In certain embodiments, the greatest local average hydrophilicity of a protein, as governed by the hydrophilicity of its adjacent amino acids, correlates with its immunogenicity and antigen-binding or immunogenicity, i.e., with a biological property of the protein.

As described in U.S. Pat. No. 4,554,101, the following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in certain embodiments, the substitution of amino acids whose hydrophilicity values are within ±2 is included, in certain embodiments, those that are within ±1 are included, and in certain embodiments, those within ±0.5 are included.

Exemplary amino acid substitutions are set forth in Table 1.

TABLE 1
Amino Acid Substitutions

| Original | Exemplary | Preferred |
|---|---|---|
| Residues | Substitutions | Substitutions |
| Ala | Val, Leu, Ile | Val |
| Arg | Lys, Gln, Asn | Lys |
| Asn | Gln | Gln |
| Asp | Glu | Glu |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, | Leu |
| | Phe, Norleucine | |
| Leu | Norleucine, Ile, | Ile |
| | Val, Met, Ala, Phe | |
| Lys | Arg, Gln, Asn, | Arg |
| | 1,4 Diamine-butyric Acid | |
| Met | Leu, Phe, Ile | Leu |
| Phe | Leu, Val, Ile, Ala, | Leu |
| | Tyr | |
| Pro | Ala | Gly |
| Ser | Thr, Ala, Cys | Thr |
| Thr | Ser | Ser |
| Trp | Tyr, Phe | Tyr |
| Tyr | Trp, Phe, Thr, Ser | Phe |
| Val | Ile, Met, Leu, Phe, | Leu |
| | Ala, Norleucine | |

A skilled artisan can determine suitable variants of the polypeptide as set forth herein using well-known techniques. In certain embodiments, one skilled in the art can identify suitable areas of the molecule that can be changed without destroying activity by targeting regions not believed to be important for activity. In certain embodiments, one can identify residues and portions of the molecules that are conserved among similar polypeptides. In certain embodiments, even areas that are important for biological activity or for structure can be subject to conservative amino acid substitutions without destroying the biological activity or without adversely affecting the polypeptide structure.

Additionally, one skilled in the art can review structure-function studies identifying residues in similar polypeptides that are important for activity or structure. In view of such a comparison, one can predict the importance of amino acid residues in a protein that correspond to amino acid residues important for activity or structure in similar proteins. One skilled in the art may opt for chemically similar amino acid substitutions for such predicted important amino acid residues.

One skilled in the art can also analyze the three-dimensional structure and amino acid sequence in relation to that structure in similar polypeptides. In view of such information, one skilled in the art can predict the alignment of amino acid residues of a polypeptide with respect to its three dimensional structure. In certain embodiments, one skilled in the art may choose not to make radical changes to amino acid residues predicted to be on the surface of the protein, since such residues may be involved in important interactions with other molecules. Moreover, one skilled in the art may generate test variants containing a single amino acid substitution at each desired amino acid residue. The variants can then be screened using activity assays known to those skilled in the art. Such variants can be used to gather information about suitable variants. For example, if it was discovered that a change to a particular amino acid residue resulted in destroyed, undesirably reduced, or unsuitable activity, variants with such a change can be avoided. In other words, based on information gathered from such routine experiments, one skilled in the art can readily determine the amino acids where further substitutions should be avoided either alone or in combination with other mutations.

Stereoisomers (e.g., D-amino acids) of the twenty conventional amino acids, non-naturally occurring amino acids such as α-,α-disubstituted amino acids, N-alkyl amino acids, lactic acid, and other unconventional amino acids may also be suitable components for polypeptides of the present invention. Examples of unconventional amino acids include but are not limited to: 4-hydroxyproline, γ-carboxyglutamate, ε-N,N,N-trimethyllysine, α-N-acetyllysine, O-phosphoserine, N-acetylserine, N-formylmethionine, 3-methylhistidine, 5-hydroxylysine, σ-N-methylarginine, and other similar amino acids and imino acids (e.g., 4-hydroxyproline). In the polypeptide notation used herein, the left-hand direction is the amino terminal direction and the right-hand direction is the carboxy-terminal direction, in accordance with standard usage and convention.

Peptide analogs are commonly used in the pharmaceutical industry as non-peptide drugs with properties analogous to those of the template peptide. These types of non-peptide compound are termed “peptide mimetics” or “peptidomimetics.” (See Fauchere, 1986, Adv. Drug Res. 15: 29; Veber and Freidinger, 1985, TINS p.392; and Evans et al., 1987, J. Med. Chem. 30: 1229, which are incorporated herein by reference for any purpose.) Such compounds are often developed with the aid of computerized molecular modeling. Peptide mimetics that are structurally similar to therapeutically useful peptides may be used to produce a similar therapeutic or prophylactic effect. Generally, peptidomimetics are structurally similar to a paradigm polypeptide (i.e., a polypeptide that has a biochemical property or pharmacological activity), but have one or more peptide linkages optionally replaced by a linkage such as: —CH₂NH—, —CH₂S—, —CH₂—CH₂—, —CH═CH—(cis and trans), —COCH₂—, —CH(OH)CH₂—, and —CH₂SO—, by methods well known in the art. Systematic substitution of one or more amino acids of a consensus sequence with a D-amino acid of the same type (e.g., D-lysine in place of L-lysine) may be used in certain embodiments to generate more stable peptides. In addition, conformationally-constrained peptides comprising a consensus sequence or a substantially identical consensus sequence variation may be generated by methods known in the art (Rizo and Gierasch, 1992, Ann. Rev. Biochem. 61: 387), incorporated herein by reference for any purpose); for example, by adding internal cysteine residues capable of forming intramolecular disulfide bridges which cyclize the peptide.

As used herein, the term “isolated polynucleotide” as used herein means a polynucleotide of genomic, cDNA, or synthetic origin or a combination thereof, which by virtue of its source the “isolated polynucleotide” (1) is not associated with all or a portion of a polynucleotide in which the “isolated polynucleotide” is found in nature, (2) is linked to a polynucleotide which it is not linked to in nature, or (3) does not occur in nature as part of a larger sequence.

Unless specified otherwise, the left-hand end of single-stranded polynucleotide sequences is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA and which are 5′ to the 5′ end of the RNA transcript are referred to as “upstream sequences”; sequence regions on the DNA strand having the same sequence as the RNA and which are 3′ to the 3′ end of the RNA transcript are referred to as “downstream sequences”.

The term “polynucleotide” as used herein means a polymeric form of nucleotides that are at least 10 bases in length. In certain embodiments, the bases may be ribonucleotides or deoxyribonucleotides or a modified form of either type of nucleotide. The term includes single and double stranded forms of DNA.

The term “oligonucleotide” as used herein includes naturally occurring, and modified nucleotides linked together by naturally occurring, and/or non-naturally occurring oligonucleotide linkages. Oligonucleotides are a polynucleotide subset generally comprising no more than 200 nucleotides. In certain embodiments, oligonucleotides are 10 to 60 nucleotides in length. In certain embodiments, oligonucleotides are 12, 13, 14, 15, 16, 17, 18, 19, or 20 to 40 bases in length. Oligonucleotides are single stranded, e.g. for use in the construction of a gene mutant using site directed mutagenesis techniques. Oligonucleotides of the invention may be sense or antisense oligonucleotides.

The term “naturally occurring nucleotides” includes deoxyribonucleotides and ribonucleotides. The term “modified nucleotides” includes nucleotides with modified or substituted sugar groups and the like. The term “oligonucleotide linkages” includes oligonucleotides linkages such as phosphate, phosphorothioate, phosphorodithioate, phosphoroselenoate, phosphorodiselenoate, phosphoroanilothioate, phoshoraniladate, phosphoroamidate, and the like. See, e.g., LaPlanche et al., 1986, Nucl. Acids Res. 14: 9081; Stec et al., 1984, J. Am. Chem. Soc. 106: 6077; Stein et al., 1988, Nucl. Acids Res. 16: 3209; Zon et al, 1991, Anti-Cancer Drug Design 6: 539; Zon et al., 1991, OLIGONUCLEOTIDES AND ANALOGUES: A PRACTICAL APPROACH, (F. Eckstein, ed.), Oxford University Press, Oxford England, pp. 87-108; Stec et al., U.S. Pat. No. 5,151,510; Uhlmann and Peyman, 1990, Chemical Reviews 90: 543, the disclosures of each of which are hereby incorporated by reference for any purpose. An oligonucleotide can include a detectable label, such as a radiolabel, a fluorescent label, an antigenic label or a hapten.

The term “agent” is used herein to denote a chemical compound, a mixture of chemical compounds, a biological macromolecule, or an extract made from biological materials.

As used herein, the terms “label” or “labeled” or “detectably labeled” refers to incorporation of a detectable marker, e.g., by incorporation of a radiolabeled amino acid or attachment to a polypeptide of biotin moieties that can be detected by marked avidin (e.g., streptavidin containing a fluorescent marker or enzymatic activity that can be detected by optical or colorimetric methods). In certain embodiments, the label or marker can also be therapeutic. Various methods of labeling polypeptides and glycoproteins can be used that are known in the art. Examples of labels for polypeptides include, but are not limited to, the following: radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I), fluorescent labels (e.g., FITC, rhodamine, lanthamide phosphors), enzymatic labels (e.g., horseradish peroxidase, β-galactosidase, luciferase, alkaline phosphatase), chemiluminescent groups, biotin, and predetermined polypeptide epitopes recognized by a secondary reporter (e.g., leucine zipper pair sequences, binding sites for secondary antibodies, metal binding domains, epitope tags). In certain embodiments, labels are attached by spacer arms of various lengths (such as —(CH₂)ₙ—, n=1-50, more preferably 1-20) to reduce steric hindrance.

The phrase “recombinant nucleic acid construct” as used herein refers to a DNA or RNA sequence that comprises a coding sequence that is operatively linked to a control sequence. A recombinant nucleic acid construct of the invention is capable of expressing a protein that is encoded by the coding sequence when introduced into a cell. A recombinant nucleic acid construct of the invention preferably comprises the nucleic acid sequence that encodes a protein as set forth in SEQ ID NO: 2, such as the nucleic acid sequence as set forth in SEQ ID NO: 1, whereby a cell contacted with the recombinant nucleic acid construct expresses FoxM1B protein. The term “operatively linked” as used herein refers to components that are in a relationship permitting them to function in their intended or conventional manner. For example, a control sequence “operatively linked” to a coding sequence is ligated thereto in such a way that expression of the coding sequence is achieved under conditions compatible with the control sequences.

In some embodiments of the invention, a recombinant nucleic acid construct is used to introduce a full-length or less than full-length FoxM1B gene into a cell. As used herein, a “full-length” FoxM1B gene refers to the wild type FoxM1B gene or a variant thereof that encodes a functional FoxM1B protein. A “less than full-length” FoxM1B gene, as used herein, refers to a truncated version of a full-length FoxM1B gene as defined herein, wherein the less than full-length FoxM1B gene encodes a functional FoxM1B protein. In a preferred embodiment, the less than full-length FoxM1B gene has a nucleotide sequence as set forth in SEQ ID NO: 1. As used herein, a “functional FoxM1B protein” is a wild type FoxM1B protein as described herein, or a variant thereof (e.g. a wild type FoxM1B protein comprising an addition, deletion, and/or substitution of at least one amino acid) that can restore proliferative potential to a cell when expressed in the cell.

The term “control sequence” as used herein refers to polynucleotide sequences that can effect the expression and processing of coding sequences to which they are ligated. The nature of such control sequences may differ depending upon the host organism. According to certain embodiments, control sequences for prokaryotes may include promoters, repressors, operators, ribosomal binding sites, and transcription termination sequences and antisense mRNA. According to certain embodiments, control sequences for eukaryotes may include promoters, enhancers and transcription termination sequences, or sequences that regulate protein degradation, mRNA degradation, nuclear localization, nuclear export, cytoplasmic retention, protein phosphorylation, protein acetylation, protein sumolation, or RNA inhibition (RNAi). In certain embodiments, “control sequences” can include leader sequences and/or fusion partner sequences. “Control sequences” are “operatively linked” to a coding sequence when the “control sequence” effects expression and processing of coding sequences to which they are ligated.

As used herein, the phrase “tissue specific promoters” refers to nucleic acid sequences that are capable of directing transcription of a coding sequence and that are activated specifically within a specific cell type. Various tissue specific promoters are known and used in the art for various types of tissues. For example, liver specific promoters that drive expression of genes in liver cells include, but are not limited to, human or mouse α1-antitrypsin, albumin promoter, serum amyloid A, transthyretin, hepatocyte nuclear factor 6, and major urinary protein (MUP).

The term “vector” is used to refer to any molecule (e.g., nucleic acid, plasmid, or virus) used to transfer coding information to a host cell or a target cell. Viral vectors suitable for the methods of the invention include those derived from, for example, an adenovirus, an adeno-associated virus, a retrovirus, a herpes simplex virus, or a vaccinia virus.

The term “expression vector” refers to a vector that is suitable for transformation of a host cell or a target cell and contains nucleic acid sequences that direct and/or control the expression of inserted heterologous nucleic acid sequences. Expression includes, but is not limited to, processes such as transcription, translation, and RNA splicing, if introns are present.

The term “host cell” is used to refer to a cell into which has been introduced, or that is capable of having introduced, a nucleic acid sequence and then of expressing a gene of interest. The term includes the progeny of the parent cell, whether or not the progeny is identical in morphology or in genetic make-up to the original parent, so long as the gene is present.

The term “transduction” is used to refer to the transfer of genes from one bacterium to another, usually by a phage. “Transduction” also refers to the acquisition and transfer of eukaryotic cellular sequences by viruses such as retroviruses.

The term “transfection” is used to refer to the uptake of foreign or exogenous DNA by a cell, and a cell has been “transfected” when the exogenous DNA has been introduced inside the cell membrane. A number of transfection techniques are well known in the art and are disclosed herein. See, e.g., Graham et al., 1973, Virology 52: 456; Sambrook et al., 2001, MOLECULAR CLONING: A LABORATORY MANUAL, 3d ed., Cold Spring Harbor Laboratory Press, Cold Spring Harbor, N.Y.; Davis et al., 1986, BASIC METHODS IN MOLECULAR BIOLOGY (Elsevier); and Chu et al., 1981, Gene 13: 197. Such techniques can be used to introduce one or more exogenous DNA moieties into suitable host cells.

The term “transformation” as used herein refers to a change in a cell's genetic characteristics, and a cell has been transformed when it has been modified to contain a new DNA. For example, a cell is transformed where it is genetically modified from its native state. Following transfection or transduction, the transforming DNA may recombine with that of the cell by physically integrating into a chromosome of the cell, may be maintained transiently as an episomal element without being replicated, or may replicate independently as a plasmid. A cell is stably transformed when the DNA is replicated with the division of the cell.

The term “pharmaceutical composition” as used herein refers to a chemical compound or composition capable of inducing a desired therapeutic effect when properly administered to a patient.

The term “therapeutically effective amount” refers to the amount of growth hormone or a compound identified in a screening method of the invention determined to produce a therapeutic response in a mammal. Such therapeutically effective amounts are readily ascertained by one of ordinary skill in the art.

As used herein, “substantially pure” means an object species that is the predominant species present (i.e., on a molar basis it is more abundant than any other individual species in the composition). In certain embodiments, a substantially purified fraction is a composition wherein the object species comprises at least about 50 percent (on a molar basis or on a weight or number basis) of all macromolecular species present. In certain embodiments, a substantially pure composition will comprise more than about 80%, 85%, 90%, 95%, or 99% of all macromolar species present in the composition. In certain embodiments, the object species is purified to essential homogeneity (wherein contaminating species cannot be detected in the composition by conventional detection methods) wherein the composition consists essentially of a single macromolecular species.

The term “patient” includes human and animal subjects.

The term “target cell” as used herein refers to a cell of particular interest that is associated with aging, premature aging, or any other condition or disease wherein the induction of FoxM1B protein expression and/or FoxM1B nuclear localization will restore proliferation potential in the cell. Preferred target cells of the invention include, but are not limited to skin cells, lung cells, intestinal epithelial cells, colon cells, testes cells, and thymus cells that undergo atrophy with aging resulting in reduction in the immune response and cancer, all of which may be associated with tissue damage, or with a disease or condition associated with aging. Target cells that specifically need restoration of proliferative potential include, but are not limited to, lung cells of patients with emphysema or respiratory distress syndrome, which cause severe problems with breathing in the elderly; proliferative basal cells of the skin, which exhibit diminished proliferation during aging; epithelial cells of the intestinal crypts that exhibit diminished proliferation during aging and whose proliferation is required to replenish the epithelial cells of the villus, which undergo programmed cell death every three days; and stem cells in the brain that exhibit diminished proliferation.

As used herein, the phrase “lung damage” refers to damage occurring to the lung tissue of a mammal that is caused by a disease or pulmonary disorder, such as emphysema, respiratory distress syndrome, or asthma, or caused by chronic or acute environmental insult, including damage due to air pollution and smoking.

As used herein, the term “autograft” refers to removal of part of an organism and its replacement in the body of the same individual. An autograft can be the introduction of autologous organs, tissue, or cells in an individual.

As used herein the term “allograft” refers to the removal of part of one individual and its replacement in the body of a different individual. An allograft is also referred to as a xenograft, heterograft, or heterologous graft. Allografts can be obtained, for example, from organ donation.

The phrase “liver cells” as used herein refers to the cells that make up a mammalian liver. Liver cells include, for example, hepatocytes, Kupffer cells, biliary epithelial cells, fenestrated endothelial cells, and cells of Ito.

The phrase “lung cells” as used herein refers to the cells that make up a mammalian lung. Lung cells include, for example, type I and type II alveolar epithelial cells, alveolar macrophages, vascular endothelium, fibroblasts, bronchiolar epithelium, clara cells, goblet cells, neuroendocrine cells, bronchiolar and vascular smooth muscle cells, and ciliated epithelial cells.

As used herein, the term “regeneration” refers to the growth or proliferation of new tissue. Regenerated tissue of the invention will have cytological, histological, and functional characteristics of normal tissue. Such characteristics can be examined by any method known in the art. For example, regenerated tissue of the invention can be examined for expression of common markers indicative of a function of a particular tissue type.

The term “cytokine” as used herein refers to molecules, such as small proteins or other biological factors, which are released by cells and have specific effects on cell-cell interaction, communication, and behavior of other cells. For example, a cytokine can be tumor necrosis factor α, interleukin 6 (IL-6), IL-1α, or IL-1β.

The term “growth factor” as used herein refers to any substance, whether made by the body or synthetically, that can function to regulate cell division and cell survival. For example, a growth factor can be growth hormone, hepatocyte growth factor, epidermal growth factor, transforming growth factor α, or a growth factor that induces Ras-MAP kinase signaling.

The term “growth hormone” refers to growth hormone from any species, including bovine, ovine, porcine, equine, and preferably human, in native-sequence or in variant form, and from any source, whether natural, synthetic, or recombinant. Preferred herein for human use is human native-sequence, mature growth hormone with or without a methionine at its N-terminus. Also preferred is recombinant human growth hormone (hGH), produced, for example, by means of recombinant DNA technology.

Human growth hormone is commercially available and known as somatrem and somatropin. Somatrem is typically used to treat children with growth failure caused by hGH deficiency. The usual weekly dosage of somatrem for children is 0.3 milligram (mg) per kilogram (kg) of body weight. Somatropin is used to treat growth failure caused by Turner's syndrome, kidney disease, or a lack of hGH. The usual weekly dosage of somatropin for children is 0.16 to 0.375 mg per kg of body weight. For adults, 0.006 mg per kg is usually taken daily and increased gradually as needed. AIDS patients experiencing dramatic weight loss are given up to 6 mg of somatropin per day depending on body weight. Somatropin and somatrem are typically administered by injection under the skin or directly into a muscle. Forms of orally administered growth hormone are also known in the art (see, for example, U.S. Pat. No. 6,239,105).

The aging process affects all of the body's cells and, consequently, all the tissues and organ systems of an individual. As used herein, “symptoms” associated with aging refers to any change caused by the aging process. The major changes associated with aging include, but are not limited to, changes in overall body shape, hair and nails, hormone production, immune responses, skin, sleep patterns, bones, muscles, joints, breast, facial features, female and male reproductive systems, heart and blood vessels, kidney function, lungs, nervous system, senses, and vital signs. Such changes can occur in response to normal aging, premature aging, or age-related diseases or disorders.

An “age-related disease” or “age-related proliferation disorder” referred to herein includes, but is not limited to emphysema and respiratory distress syndrome, cancer, stomach and intestinal ulcers, degenerative diseases of the brain, liver, lung, and intestine.

The phrase “premature aging” as used herein refers to any process that accelerates the aging process in a mammal. Premature aging can be caused by, for example, a disease, lifestyle, or environment. Diseases that cause premature aging include, but are not limited to, Hutchinson-Gilford Progeria and Werner Syndrome.

Mouse genetic studies have demonstrated that increased p53 activity results in premature aging and early aging-associated phenotypes (Tyner et al., 2002, Nature 415: 45-53). The potential for increased FoxM1B expression to mediate diminished p53 protein levels in regenerating hepatocytes of old-aged TTR-FoxM1B TG mice was examined as described herein. Prior to hepatocyte DNA replication (24 to 36 hours post PHx), Western blot analysis revealed a 50-70% reduction in p53 protein levels in regenerating livers of old-aged TTR-FoxM1B TG mice compared to old-aged WT mice. Coincident with the reduction of p53 protein levels, a 50% reduction in p21^{Cip1} protein expression prior to S-phase in regenerating livers of old-aged TTR-FoxM1B TG mice was observed. These liver regeneration studies indicate that maintaining FoxM1B levels caused diminished expression of p53 and p21^{Cip1} proteins during the G1 to S-phase transition in old-aged TTR FoxM1B TG mice, which is consistent with preventing reduced proliferating associated with an aging phenotype.

Proliferation defects during aging leads to diminished muscle mass and thinning of the skin, which is associated with a progressive decline in growth hormone (GH) secretion and serum GH binding protein. GH treated old aged mice exhibited an increase in regenerating hepatocyte DNA replication and mitosis to levels found in young regenerating liver. Furthermore, as demonstrated herein, GH restores hepatocyte proliferation in regenerating liver of old aged mice by increasing expression and nuclear localization of FoxM1B. This suggests that GH mediates increased hepatocyte proliferation by restoring FoxM1B expression in regenerating livers of old aged mice.

As discussed herein, short term GH administration can be used to stimulate FoxM1B expression and cell proliferation in diseased tissues that exhibit defects in tissue regeneration. Also, short term GH administration can be effective in live donor transplants of organs, tissues, or cells to a recipient. These are donors that give a recipient an organ, a portion of an organ, tissue, or cells that require regeneration of the organ or tissue in the recipient. Regeneration of a portion of the organ or the tissue may also be required in the donor. Advantageously, GH can be administered to donor and recipient several days prior to a transplantation procedure, thereby stimulating regeneration in the organ of the live donor and in the recipient and allow better prognosis for both patients. The Examples herein demonstrate that GH administration is a useful therapeutic intervention that enhances organ and tissue regeneration through increased expression and nuclear localization of FoxM1B.

The invention provides methods for treating patients diagnosed with an age-related disease or proliferation disorder, a disease or condition associated with premature aging, or tissue damage. The invention also provides methods for treating and preventing symptoms associated with normal aging and premature aging. In these aspects of the invention, patients are treated with growth hormone in a medically acute manner rather than a medically chronic manner, that is, the treatment has a duration that is limited by the nature and extent of the disease, injury or damage and terminates upon detection of positive response in the patient. Preferably, the invention provides transient nuclear localization of FoxM1B protein in the patients treated with a growth factor or a cytokine in a medically acute manner. As used herein, “transient nuclear localization” refers to non-permanent localization of FoxM1B protein in the nucleus of a cell. For example, FoxM1B protein can be induced to localize in the nucleus of a hepatocyte by exposure to growth hormone, while the FoxM1B protein is not detectable in the nucleus once exposure to growth hormone is discontinued.

For human growth hormone (hGH), a suitable dosage for human administration ranges from 0.001 mg to about 0.2 mg per kg of body weight per day. Generally, therapeutically effective daily dosages of hGH will be from about 0.05 mg to about 0.2 mg per kg of body weight per day. For most patients, doses of from 0.07 to 0.15 mg/kg, in one or more applications per day, is effective to obtain the desired result. In an alternative approach, hGH may be administered less frequently, particularly where formulated in a timed-release form, e.g., every other day or every third day for certain indications.

During treatment with hGH, patients can be monitored by the assays described and known in the art for improvement in organ, tissue, or cell function. When function is restored to a level that resembles that of a healthy organ, tissue, or cell, suggesting that the regeneration process is sufficient, growth hormone administration is discontinued. Thus, it is an advantage of the invention that patients are not chronically exposed to growth hormone.

In certain embodiments, the invention provides methods for treating an age-related disease or proliferation disorder, a disease or condition associated with premature aging, or tissue damage in mammals by inducing FoxM1B protein to translocate from the cytoplasm to the nucleus in target cells, where it potentiates transcription of many cell cycle promotion genes and stimulates cellular proliferation. In a particular embodiment, the mammal is treated with growth hormone to induce nuclear localization of FoxM1B protein. One of skill in the art will recognize that the methods of the invention will be useful when practiced in vivo, in vitro, or ex vivo.

In particular embodiments, the invention provides methods for restoring proliferative potential of target cells comprising introducing a recombinant nucleic acid construct that comprises SEQ ID NO: 1 into the target cells. As used herein, “proliferative potential” refers to the ability of a cell to proliferate in response to certain conditions or factors that typically induce the cell to divide.

The nucleic acid sequence set forth in SEQ ID NO: 1 is a 2737 nucleotide FoxM1B cDNA fragment that encodes the entire FoxM1B protein. Expression of FoxM1B increases during cellular proliferation through stabilization of the FoxM1B mRNA. Unlike the endogenous FoxM1B mRNA, when the 2737 nucleotide FoxM1B cDNA fragment transgene (SEQ ID NO: 1, as shown in FIG. 1) is expressed as RNA in non-dividing cells it is stable in non-dividing cells and will accumulate in non-dividing cells (Ye et al., 1999, Mol. Cell Biol., 19: 8570-8580). The deletion of the terminal 972 nucleotides at the 3′ end of the FoxM1B cDNA therefore contains sequences that mediate RNA degradation of FoxM1B mRNA in non-dividing cells. As described herein, expression of FoxM1B is reduced in cells during the aging process. Introducing SEQ ID NO: 1 into cells in which endogenous FoxM1B protein expression is reduced or absent will restore the cells' ability to respond to proliferation conditions or factors, such as injuries, growth factors, and cytokines.

In some embodiments, the invention provides methods for treating or preventing symptoms associated with aging comprising the step of introducing a recombinant nucleic acid construct that comprises SEQ ID NO: 1 into target cells, thereby restoring proliferative potential of the target cells; methods for preventing or amelioriating the effects of an age-related disease or age-related proliferation disorder in a patient comprising the step of introducing a recombinant nucleic acid construct that comprises SEQ ID NO: 1 into target cells, thereby restoring proliferative potential of the target cells; methods for treating diseases or disorders associated with premature aging comprising the step of introducing a recombinant nucleic acid construct that comprises SEQ ID NO: 1 into the target cell, thereby restoring proliferative potential of the target cells; and methods for preventing or ameliorating the effects of lung injury comprising the step of introducing a recombinant nucleic acid construct that comprises SEQ ID NO: 1 into lung cells, thereby restoring proliferative potential of the lung cells. In one aspect, FoxM1B protein expression and/or nuclear localization in the target cell or lung cell by contacting the cells with a growth factor or a cytokine, as described herein.

In other embodiments, the invention provides methods of screening for compounds that induce expression of FoxM1B protein, induce nuclear localization of FoxM1B protein, or induce both expression and nuclear localization of FoxM1B protein. Compounds identified in these screens can be used in the methods of treating an age-related disease or proliferation disorder, a disease or condition associated with premature aging, or tissue damage as discussed herein. Alternatively, compounds identified in these screens can be used in the methods of treating or preventing symptoms associated with normal aging or premature aging.

Screening for compounds that induce expression of FoxM1B protein can be accomplished, for example, with cells that comprise a full-length or less than full-length FoxM1B gene but do not express FoxM1B protein under normal culture conditions. Such cells can include, for example, hepatocytes from aged individuals, host cells comprising a FoxM1B gene as discussed below, or quiescent cells that do not express FoxM1B protein.

The method of screening for compounds that induce expression of FoxM1B in mammalian cells can be accomplished as follows: (a) contacting a plurality of cells that comprise a full-length or less than full-length FoxM1B gene, wherein the FoxM1B protein is not expressed under normal culture conditions, with a candidate compound in the presence of human growth hormone; (b) contacting a plurality of cells that comprise the FoxM1B gene, wherein the FoxM1B protein is not expressed under normal culture conditions, with the candidate compound in the absence of human growth hormone; and (c) assaying FoxM1B expression and localization in the cells from step (a) and step (b); wherein a candidate compound is selected if FoxM1B is localized in the nuclei of cells from step (a) and in the cytoplasm of cells from step (b). Said assay can be a direct assay for nuclear localization of FoxM1B, or can be an indirect assay for the presence or activity of a gene product expressed as a consequence of FoxM1B translocation into the nucleus from the cytoplasm.

The inventive methods of screening for compounds that induce nuclear localization of FoxM1B protein can be accomplished by contacting a cell with a candidate compound, wherein the cell expresses FoxM1B protein, and examining localization of FoxMIB protein in the cell. The candidate compound is selected if FoxM1B protein is localized in the nucleus of the cell. In certain embodiments, the Fox M1B is endogenous, i.e., it comprises the genomic DNA complement of the cell. In other embodiments, the FoxM1B is exogenous and is experimentally introduced, most preferably as a recombinant nucleic acid construct of the invention encoding most preferably a heterologous Fox M1B gene, i.e., from a mammalian species different from the host cell species.

The methods of screening for compounds that induce both expression and nuclear localization of FoxM1B protein in a manner similar to that of growth hormone, can be accomplished as follows: (a) contacting a plurality of cells that comprise a full-length or less than full-length FoxM1B gene, wherein the FoxM1B protein is not expressed under normal culture conditions, with a candidate compound; and (b) assaying FoxM1B expression and FoxM1B localization in the cells from step (a); wherein a candidate compound is selected if FoxM1B is expressed and localized in the nuclei of cells contacted with the compound in a manner similar to the pattern observed in cells contacted with growth hormone. In alternative embodiments, the cells of step (a) can be contacted with growth hormone prior to assay in step (b).

Assaying for nuclear localization and expression of FoxM1B protein can be accomplished by any method known the art. For example, immunohistochemistry using detectably-labeled primary anti-FoxM1B antibodies, or unlabeled primary anti-FoxM1B and detectably-labeled secondary antibodies (for example, labeled with fluorescent markers, such as fluorescein isothiocyanate, FITC), can be used to visualize FoxM1B protein localization, inter alia, by fluorescence microscopy. Alternative labels, such as radioactive, enzymatic and hapten labels, are within the scope of this invention.

In certain embodiments, methods of the invention comprise the step of expressing FoxM1B protein in a host cell or a target cell by introducing into the cell a recombinant nucleic acid construct of the invention. According to such embodiments, the cells are transformed with the recombinant nucleic acid construct using any method for introducing polynucleotides into a host cell or a target cell, including, for example packaging the polynucleotide in a virus (or into a viral vector) and transducing a host cell or a target cell with the virus (or vector), or by transfection procedures known in the art, as exemplified by U.S. Pat. Nos. 4,399,216, 4,912,040, 4,740,461, and 4,959,455 (which patents are hereby incorporated herein by reference for any purpose). In certain embodiments, the transformation procedure used may depend upon the cell to be transformed. Methods for introduction of heterologous polynucleotides into mammalian cells are well known in the art and include, but are not limited to, dextran-mediated transfection, calcium phosphate precipitation, polybrene mediated transfection, protoplast fusion, electroporation, encapsulation of the polynucleotide(s) in liposomes, mixing nucleic acid with positively-charged lipids, and direct microinjection of the DNA into cells and cell nuclei.

Recombinant nucleic acid constructs of the invention typically comprise a nucleic acid molecule encoding all or a functional portion of the amino acid sequence of FoxM1B protein that is inserted into an appropriate expression vector using conventional recombinant genetic techniques. Preferably, the recombinant nucleic acid construct of the invention comprises a nucleic acid sequence that encodes a protein as set forth in SEQ ID NO: 2. The vector is typically selected to be functional in the particular host cell or target cell employed (i.e., the vector is compatible with the host cell or the target cell machinery, permitting amplification and/or expression of the gene). For a review of expression vectors, see Nolan and Shatzman, 1998, Curr. Opin. Biotechnol. 9:447-450.

Typically, expression vectors used in any of the host cells or target cells contain sequences for vector maintenance and for cloning and expression of exogenous nucleotide sequences. Such sequences, collectively referred to as “flanking sequences” in certain embodiments will typically include one or more of the following nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation signal sequence, a polylinker region comprising one or a plurality of restriction endonuclease sites for inserting nucleic acid encoding the polypeptide to be expressed, and a selectable marker element. Each of these sequences is discussed below.

Flanking sequences may be homologous (i.e., from the same species and/or strain as the host cell or the target cell), heterologous (i.e., from a species other than the host cell or the target cell species or strain), hybrid (i.e., a combination of flanking sequences from more than one source), synthetic or native. As such, the source of a flanking sequence may be any prokaryotic or eukaryotic organism, any vertebrate or invertebrate organism, or any plant, provided that the flanking sequence is functional in, and can be activated by, the host cell or the target cell machinery.

Flanking sequences useful in the vectors of this invention may be obtained by any of several methods well known in the art. Typically, flanking sequences useful herein will have been previously identified by mapping and/or by restriction endonuclease digestion and can thus be isolated from the proper tissue source using the appropriate restriction endonucleases. In some cases, the full nucleotide sequence of a flanking sequence may be known. The flanking sequence also may be synthesized using the methods described herein for nucleic acid synthesis or cloning.

Where all or only a portion of the flanking sequence is known, it may be obtained using in vitro amplification methods such as polymerase chain reaction (PCR) and/or by screening a genomic library with a suitable oligonucleotide and/or flanking sequence fragment from the same or another species. Where the flanking sequence is not known, a fragment of DNA containing a flanking sequence may be isolated from a larger piece of DNA that may contain, for example, a coding sequence or even another gene or genes. Isolation may be accomplished by restriction endonuclease digestion to produce the proper DNA fragment followed by isolation using agarose gel purification, Qiagen® column chromatography (Chatsworth, Calif.), or other methods known to the skilled artisan. The selection of suitable enzymes to accomplish this purpose is readily apparent to one of ordinary skill in the art.

Optionally, the vector may contain a “tag”-encoding sequence, i.e., an oligonucleotide molecule located at the 5′ or 3′ end of the FoxM1B polypeptide coding sequence, wherein such an oligonucleotide sequence encodes polyhis (such as hexaHis), or another “tag” for which commercially available antibodies exist, such as FLAG, HA (hemaglutinin influenza virus), or myc. This tag oligonucleotide is typically ligated to the coding sequence “in frame” so that the tag is fused to the polypeptide upon expression of the polypeptide, and can serve as a means for affinity purification of the FoxM1B polypeptide from the host cell or the target cell. Affinity purification can be accomplished, for example, by column chromatography using antibodies against the tag as an affinity matrix. Optionally, the tag can subsequently be removed from the purified FoxM1B polypeptide by various means such as using certain peptidases for cleavage. In preferred embodiments of such vectors permitting removal of the tag, a protease cleavage site is included in the tag sequence in a position between the tag and polypeptide amino acid sequences when the tagged polypeptide is expressed.

In some cases, for example where glycosylation is desired in a eukaryotic host cell expression system, various presequences can be manipulated to improve glycosylation or yield. For example, the peptidase cleavage site of a particular signal peptide can be altered, or pro-sequences added, which also may affect glycosylation. The final protein product may have, in the −1 position (relative to the first amino acid of the mature protein) one or more additional amino acids incident to expression, which may not have been totally removed. For example, the final protein product may have one or two amino acid residues found in the peptidase cleavage site, attached to the amino-terminus. Alternatively, use of some enzyme cleavage sites may result in a slightly truncated yet active form of the desired polypeptide, if the enzyme cuts at such area within the mature polypeptide.

A transcription termination sequence is typically located 3′ to the end of a polypeptide-coding region and serves to terminate transcription. Usually, a transcription termination sequence in prokaryotic cells is a G-C rich fragment followed by a poly-T sequence. While the sequence is easily cloned from a library or even purchased commercially as part of a vector, it can also be readily synthesized using methods for nucleic acid synthesis such as those described herein. In eukaryotes, the sequence AAUAAA (SEQ ID NO: 3) functions both as a transcription termination signal and as a poly A signal required for endonuclease cleavage followed by the addition of poly A residues (usually consisting of about 200 A residues).

A selectable marker gene element encodes a protein necessary for the survival and growth of a host cell or a target cell grown in a selective culture medium. Typical selection marker genes encode proteins that (a) confer resistance to antibiotics or other toxins, e.g., ampicillin, tetracycline, or kanamycin for prokaryotic host cells; (b) complement auxotrophic deficiencies of the cell; or (c) supply critical nutrients not available from complex media. Preferred selectable markers are the kanamycin resistance gene, the ampicillin resistance gene, and the tetracycline resistance gene. A bacterial neomycin resistance gene can also be used most advantageously for selection in both prokaryotic and eukaryotic cells.

The expression and cloning vectors of the present invention will typically contain a promoter that is recognized by the host organism and operatively linked to nucleic acid encoding the FoxM1B protein. Promoters are untranscribed sequences located upstream (i.e., 5′) to the start codon of a structural gene (generally within about 100 to 1000 bp) that control transcription of the structural gene. Promoters are conventionally grouped into one of two classes: inducible promoters and constitutive promoters. Inducible promoters initiate increased levels of transcription from DNA under their control in response to some change in culture conditions, such as the presence or absence of a nutrient or a change in temperature. Constitutive promoters, on the other hand, initiate continual gene product production; that is, there is little or no experimental control over gene expression. A large number of promoters, recognized by a variety of potential host cells or target cells, are well known. A suitable promoter is operatively linked to the DNA encoding FoxM1B protein by removing the promoter from the source DNA by restriction enzyme digestion or amplifying the promoter by polymerase chain reaction and inserting the desired promoter sequence into the vector.

Suitable promoters for use with mammalian cells are well known and include, but are not limited to, those obtained from the genomes of eukaryotic viruses such as polyoma virus, fowlpox virus, adenovirus (such as Adenovirus 2), bovine papilloma virus, avian sarcoma virus, cytomegalovirus, retroviruses, hepatitis-B virus and most preferably Simian Virus 40 (SV40). Other suitable mammalian promoters include heterologous mammalian promoters, for example, heat-shock promoters and the actin promoter.

Particular promoters useful in the practice of the recombinant expression vectors of the invention include, but are not limited to: the SV40 early promoter region (Bernoist and Chambon, 1981, Nature 290: 304-10); the CMV promoter; the promoter contained in the 3′ long terminal repeat of Rous sarcoma virus (Yamamoto, et al., 1980, Cell 22: 787-97); the herpes thymidine kinase promoter (Wagner et al., 1981, Proc. Natl. Acad. Sci. U.S.A. 78: 1444-45); and the regulatory sequences of the metallothionine gene (Brinster et al., 1982, Nature 296: 39-42). Also of interest are the following animal transcriptional control regions, which exhibit tissue specificity and have been utilized in transgenic animals: the elastase I gene control region that is active in pancreatic acinar cells (Swift et al., 1984, Cell 38: 639-46; Ornitz et al., 1986, Cold Spring Harbor Symp. Quant. Biol. 50: 399-409; MacDonald, 1987, Hepatology 7: 425-515); the insulin gene control region that is active in pancreatic beta cells (Hanahan, 1985, Nature 315: 115-22); the mouse mammary tumor virus control region that is active in testicular, breast, lymphoid and mast cells (Leder et al., 1986, Cell 45: 485-95); the beta-globin gene control region that is active in myeloid cells (Mogram et al., 1985, Nature 315: 338-40; Kollias et al., 1986, Cell 46: 89-94); the myelin basic protein gene control region that is active in oligodendrocyte cells in the brain (Readhead et al., 1987, Cell 48: 703-12); the myosin light chain-2 gene control region that is active in skeletal muscle (Sani, 1985, Nature 314: 283-86); the gonadotropic releasing hormone gene control region that is active in the hypothalamus (Mason et al., 1986, Science 234: 1372-78); and most particularly the immunoglobulin gene control region that is active in lymphoid cells (Grosschedl et al., 1984, Cell 38: 647-58; Adames et al., 1985, Nature 318: 533-38; Alexander et al., 1987, Mol. Cell Biol. 7:1436-44).

Preferably, the promoter of a recombinant nucleic acid construct of the invention is active in the tissue from which a target or host cell is derived. For example, if the cell is a liver cell, one could advantageously use the albumin gene control region (Pinkert et al., 1987, Genes and Devel. 1: 268-76); the alpha-feto-protein gene control region (Krumlauf et al., 1985, Mol. Cell Biol. 5: 1639-48; Hammer et al., 1987, Science 235: 53-58); or the alpha 1-antitrypsin gene control region (Kelsey et al., 1987, Genes and Devel. 1: 161-71), all of which are active in the liver.

The vectors of the invention can also contain an enhancer sequence that increases transcription in higher eukaryotic cells of nucleic acid encoding FoxM1B protein. Enhancers are cis-acting elements of DNA, are usually about 10-300 bp in length, and act on promoters to increase transcription. Enhancers are relatively orientation- and position-independent. They have been found within introns as well as within several kilobases both 5′ and 3′ to the transcription unit. Several enhancer sequences available from mammalian genes are known (e.g., enhancers from globin, elastase, albumin, alpha-feto-protein, insulin, transthyretin, and HNF-6 genes). An enhancer from a virus also can be used to increase expression of a gene. The SV40 enhancer, the cytomegalovirus early promoter enhancer, the polyoma enhancer, and adenovirus enhancers are exemplary enhancing elements for the activation of eukaryotic promoters. While an enhancer may be spliced into the vector at a position 5′ or 3′ to a nucleic acid molecule, it is typically located at a site 5′ from the promoter.

An origin of replication is typically a part of prokaryotic expression vectors, particularly those that are commercially available, and the origin aids in replication and amplification of the vector in a host cell or a target cell. If the vector of choice does not contain an origin of replication site, one may be chemically synthesized based on a known sequence, and ligated into the vector. For example, the origin of replication from the plasmid pBR322 (New England Biolabs, Beverly, Mass.) is suitable for most gram-negative bacteria, and various replication origins (e.g., from viruses of eukaryotes such as SV40, polyoma, adenovirus, vesicular stomatitus virus (VSV), or papillomaviruses such as HPV or BPV) are useful for cloning vectors in mammalian cells. Generally, a mammalian origin of replication is not needed for mammalian expression vectors (for example, the SV40 origin is often used only because it also contains the early promoter).

Expression vectors of the invention may be constructed from a convenient starting vector such as a commercially available vector. Such vectors may or may not contain all of the desired flanking sequences. Where one or more of the flanking sequences described herein are not already present in the vector, they may be individually obtained and ligated into the vector. Methods used for obtaining each of the flanking sequences are well known to one skilled in the art.

After the vector has been constructed and a nucleic acid molecule encoding FoxM1B protein has been inserted into the proper site of the vector, the completed vector may be inserted into a suitable host cell or a target cell for amplification and/or polypeptide expression. The transformation of an expression vector encoding FoxM1B protein into a selected host cell or target cell may be accomplished by well-known methods including methods such as transfection, infection, calcium chloride, electroporation, microinjection, lipofection, DEAE-dextran method, or other known techniques as described above. The method selected will in part be a function of the type of host cell or target cell to be used. These methods and other suitable methods are well known to the skilled artisan, and are set forth, for example, in Sambrook et al., 2001, MOLECULAR CLONING: A LABORATORY MANUAL, 3d ed., Cold Spring Harbor Laboratory Press, Cold Spring Harbor, N.Y.

A host cell or target cell, when cultured under appropriate conditions, synthesizes a FoxM1B protein that can subsequently be collected from the culture medium (if the host cell or target cell secretes it into the medium) or directly from the host cell or target cell producing it (if it is not secreted) if collection of the protein is desired. Selection of an appropriate host cell will depend upon a number of different factors, such as desired expression levels, polypeptide modifications that are desirable or necessary for activity (such as glycosylation or phosphorylation) and ease of folding into a biologically-active molecule.

Mammalian cell lines available as hosts for expression are well known in the art and include, but are not limited to, many immortalized cell lines available from the American Type Culture Collection (ATCC), such as Chinese hamster ovary (CHO) cells, HeLa cells, baby hamster kidney (BHK) cells, monkey kidney cells (COS), human hepatocellular carcinoma cells (e.g., Hep G2), and a number of other cell lines. In certain embodiments, cell lines may be selected through determining which cell lines have high expression levels of FoxM1B protein.

Selection of an appropriate target cell will also depend on the various factors discussed above for selection of an appropriate host cell. In addition, a target cell can be selected based on the disease or condition that affects a patient who is to be treated by methods of the invention. For example, if a patient has a lung injury, a lung cell can be chosen as the appropriate target cell. A target cell can be, for example, a cell from or in the patient himself or a cell from a genetically suitable donor. A “genetically suitable donor” is a donor whose tissues present a low likelihood of being rejected by the recipient once introduced or transplanted.

Alternatively, expression of FoxM1B polypeptide in a cell can be increased, or caused, by increasing, or causing, expression of a gene or genes (e.g., transcription factors) and/or decreasing the expression of a gene or genes (e.g., transcriptional repressors) in a manner which results in de novo or increased FoxM1B polypeptide production from the cell's endogenous FoxM1B gene. This method includes introducing a non-naturally occurring polypeptide (e.g., a polypeptide comprising a site-specific DNA binding domain fused to a transcriptional factor domain) into the cell such that de novo or increased FoxM1B polypeptide production from the cell's endogenous FoxM1B gene results.

The present invention further relates to DNA constructs useful in the method of altering expression of a target gene. In certain embodiments, the exemplary DNA constructs comprise: (a) one or more targeting sequences, (b) a regulatory sequence, (c) an exon, and (d) an unpaired splice-donor site. The targeting sequence in the DNA construct directs the integration of elements (a)-(d) into a target gene in a cell such that the elements (b)-(d) are operatively linked to sequences of the endogenous target gene. In another embodiment, the DNA constructs comprise: (a) one or more targeting sequences, (b) a regulatory sequence, (c) an exon, (d) a splice-donor site, (e) an intron, and (f) a splice-acceptor site, wherein the targeting sequence directs the integration of elements (a)-(f) such that the elements of (b)-(f) are operatively linked to the endogenous gene. The targeting sequence is homologous to the preselected site in the cellular chromosomal DNA with which homologous recombination is to occur. In the construct, the exon is generally 3′ of the regulatory sequence and the splice-donor site is 3′ of the exon.

If the sequence of a particular gene is known, such as the nucleic acid sequence of FoxM1B polypeptide presented herein, a DNA fragment that is complementary to a selected region of the gene can be synthesized or otherwise obtained, such as by appropriate restriction of the native DNA at specific recognition sites bounding the region of interest. This fragment serves as a targeting sequence upon insertion into the cell and will hybridize to its homologous region within the genome. If this hybridization occurs during DNA replication, this DNA fragment, and any additional sequence attached thereto, will be incorporated into the newly synthesized daughter strand of DNA. The present invention, therefore, includes nucleotides encoding a FoxM1B polypeptide, which nucleotides may be used as targeting sequences.

In certain embodiments, the invention provides pharmaceutical compositions comprising a therapeutically effective amount of a compound that induces FoxM1B expression, nuclear localization, or both expression and nuclear localization in mammalian cells together with a pharmaceutically acceptable diluent, carrier, solubilizer, emulsifier, preservative and/or adjuvant. In other embodiments, the invention provides pharmaceutical compositions that comprise a therapeutically effective amount of a compound that induces FoxM1B expression in mammalian cells and also induces FoxM1B protein to translocate into the nucleus of mammalian cells together with a pharmaceutically acceptable diluent, carrier, solubilizer, emulsifier, preservative and/or adjuvant. Such compounds are preferably identified in screening methods of the invention.

Acceptable formulation materials preferably are nontoxic to recipients at the dosages and concentrations employed. The pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. Suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides, disaccharides, and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20 and polysorbate 80, Triton, trimethamine, lecithin, cholesterol, or tyloxapal); stability enhancing agents (such as sucrose or sorbitol); tonicity enhancing agents (such as alkali metal halides, preferably sodium or potassium chloride, mannitol, or sorbitol); delivery vehicles; diluents; excipients and/or pharmaceutical adjuvants. See, for example, REMINGTON'S PHARMACEUTICAL SCIENCES, 18^{th} Edition, (A. R. Gennaro, ed.), 1990, Mack Publishing Company.

Optimal pharmaceutical compositions can be determined by one skilled in the art depending upon, for example, the intended route of administration, delivery format and desired dosage. See, for example, REMINGTON'S PHARMACEUTICAL SCIENCES, Id. Such compositions may influence the physical state, stability, rate of in vivo release and rate of in vivo clearance of the antibodies of the invention.

The primary vehicle or carrier in a pharmaceutical composition may be either aqueous or non-aqueous in nature. For example, a suitable vehicle or carrier may be water for injection, physiological saline solution or artificial cerebrospinal fluid, possibly supplemented with other materials common in compositions for parenteral administration. Neutral buffered saline or saline mixed with serum albumin are further exemplary vehicles. Pharmaceutical compositions can comprise Tris buffer of about pH 7.0-8.5, or acetate buffer of about pH 4.0-5.5, which may further include sorbitol or a suitable substitute therefor. Pharmaceutical compositions of the invention may be prepared for storage by mixing the selected composition having the desired degree of purity with optional formulation agents (REMINGTON'S PHARMACEUTICAL SCIENCES, Id.) in the form of a lyophilized cake or an aqueous solution. Further, the FoxM1B-inducing product may be formulated as a lyophilizate using appropriate excipients such as sucrose.

Formulation components are present in concentrations that are acceptable to the site of administration. Buffers are advantageously used to maintain the composition at physiological pH or at a slightly lower pH, typically within a pH range of from about 5 to about 8.

The pharmaceutical compositions of the invention can be delivered parenterally. When parenteral administration is contemplated, the therapeutic compositions for use in this invention may be in the form of a pyrogen-free, parenterally acceptable aqueous solution comprising FoxM1B protein or the desired compound identified in a screening method of the invention in a pharmaceutically acceptable vehicle. A particularly suitable vehicle for parenteral injection is sterile distilled water in which the compound identified in a screening method of the invention or FoxM1B protein is formulated as a sterile, isotonic solution, appropriately preserved. Preparation can involve the formulation of the desired molecule with an agent, such as injectable microspheres, bio-erodible particles, polymeric compounds (such as polylactic acid or polyglycolic acid), beads or liposomes, that may provide controlled or sustained release of the product which may then be delivered via a depot injection. Formulation with hyaluronic acid has the effect of promoting sustained duration in the circulation. Implantable drug delivery devices may be used to introduce the desired molecule.

Administering FoxM1B protein to a patient can be used for short-term stimulation of target cell proliferation, for example, in a recipient of a tissue or organ transplant. In addition, FoxM1B protein can be administered to a tissue or organ donor after the tissue, organ, or a portion thereof is removed to stimulate tissue or organ regeneration to reestablish organ function.

The compositions may be formulated for inhalation. In these embodiments, a compound identified in a screening method of the invention or FoxM1B protein is formulated as a dry powder for inhalation, or inhalation solutions may also be formulated with a propellant for aerosol delivery, such as by nebulization. Pulmonary administration is further described in PCT Application No. PCT/US94/001875, which describes pulmonary delivery of chemically modified proteins and is incorporated by reference.

The pharmaceutical compositions of the invention can be delivered through the digestive tract, such as orally. The preparation of such pharmaceutically acceptable compositions is within the skill of the art. FoxM1B protein or compounds of the invention that are administered in this fashion may be formulated with or without those carriers customarily used in the compounding of solid dosage forms such as tablets and capsules. A capsule may be designed to release the active portion of the formulation at the point in the gastrointestinal tract when bioavailability is maximized and pre-systemic degradation is minimized. Additional agents can be included to facilitate absorption of the FoxM1B protein or compound identified in a screening method of the invention. Diluents, flavorings, low melting point waxes, vegetable oils, lubricants, suspending agents, tablet disintegrating agents, and binders may also be employed.

A pharmaceutical composition may involve an effective quantity of FoxM1B protein or a compound identified in a screening method of the invention in a mixture with non-toxic excipients that are suitable for the manufacture of tablets. By dissolving the tablets in sterile water, or another appropriate vehicle, solutions may be prepared in unit-dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions are evident to those skilled in the art, including formulations involving FoxM1B protein or compounds of the invention in sustained- or controlled-delivery formulations. Techniques for formulating a variety of other sustained- or controlled-delivery means, such as liposome carriers, bio-erodible microparticles or porous beads and depot injections, are also known to those skilled in the art. See, for example, PCT Application No. PCT/US93/00829, which describes the controlled release of porous polymeric microparticles for the delivery of pharmaceutical compositions. Sustained-release preparations may include semipermeable polymer matrices in the form of shaped articles, e.g. films, or microcapsules, polyesters, hydrogels, polylactides (U.S. Pat. No. 3,773,919 and EP 058,481), copolymers of L-glutamic acid and gamma ethyl-L-glutamate (Sidman et al., 1983, Biopolymers 22: 547-556), poly (2-hydroxyethyl-methacrylate) (Langer et al., 1981, J. Biomed. Mater. Res. 15: 167-277) and Langer, 1982, Chem. Tech. 12: 98-105), ethylene vinyl acetate (Langer et al., id.) or poly-D(−)-3-hydroxybutyric acid (EP 133,988). Sustained release compositions may also include liposomes, which can be prepared by any of several methods known in the art. See e.g., Eppstein et al., 1985, Proc. Natl. Acad. Sci. USA 82: 3688-3692; EP 036,676; EP 088,046 and EP 143,949.

The pharmaceutical composition to be used for in vivo administration typically is sterile. In certain embodiments, this may be accomplished by filtration through sterile filtration membranes. In certain embodiments, where the composition is lyophilized, sterilization using this method may be conducted either prior to or following lyophilization and reconstitution. In certain embodiments, the composition for parenteral administration may be stored in lyophilized form or in a solution. In certain embodiments, parenteral compositions generally are placed into a container having a sterile access port, for example, an intravenous solution bag or vial having a stopper pierceable by a hypodermic injection needle.

Once the pharmaceutical composition of the invention has been formulated, it may be stored in sterile vials as a solution, suspension, gel, emulsion, solid, or as a dehydrated or lyophilized powder. Such formulations may be stored either in a ready-to-use form or in a form (e.g., lyophilized) that is reconstituted prior to administration.

The present invention is directed to kits for producing a single-dose administration unit. Kits according to the invention may each contain both a first container having a dried protein compound identified in a screening method of the invention and a second container having an aqueous formulation, including for example single and multi-chambered pre-filled syringes (e.g., liquid syringes, lyosyringes or needle-free syringes).

The effective amount of a pharmaceutical composition of the invention to be employed therapeutically will depend, for example, upon the therapeutic context and objectives. One skilled in the art will appreciate that the appropriate dosage levels for treatment, according to certain embodiments, will thus vary depending, in part, upon the molecule delivered, the indication for which the pharmaceutical composition is being used, the route of administration, and the size (body weight, body surface or organ size) and/or condition (the age and general health) of the patient. A clinician may titer the dosage and modify the route of administration to obtain the optimal therapeutic effect. Typical dosages range from about 0.1 μg/kg to up to about 100 mg/kg or more, depending on the factors mentioned above. In certain embodiments, the dosage may range from 0.1 μg/kg up to about 100 mg/kg; or 1 μg/kg up to about 100 mg/kg; or 5 μg/kg up to about 100 mg/kg.

The dosing frequency will depend upon the pharmacokinetic parameters of the FoxM1B protein or compound identified in a screening method of the invention in the formulation. For example, a clinician administers the composition until a dosage is reached that achieves the desired effect. The composition may therefore be administered as a single dose, or as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. Appropriate dosages may be ascertained through use of appropriate dose-response data.

Administration routes for the pharmaceutical compositions of the invention include orally, through injection by intravenous, intraperitoneal, intracerebral (intra-parenchymal), intracerebroventricular, intramuscular, intra-ocular, intraarterial, intraportal, or intralesional routes; by sustained release systems or by implantation devices. The pharmaceutical compositions may be administered by bolus injection or continuously by infusion, or by implantation device. The pharmaceutical composition also can be administered locally via implantation of a membrane, sponge or another appropriate material onto which the desired molecule has been absorbed or encapsulated. Where an implantation device is used, the device may be implanted into any suitable tissue or organ, and delivery of the desired molecule may be via diffusion, timed-release bolus, or continuous administration.

In certain embodiments, it may be desirable to use FoxM1B protein, FoxM1B encoding recombinant nucleic acid constructs or pharmaceutical compositions of compounds identified in a screening method of the invention in an ex vivo manner. In such instances, cells, tissues or organs that have been removed from the patient are exposed to pharmaceutical compositions of the invention or a recombinant nucleic acid construct of the invention encoding FoxM1B protein after which the cells, tissues and/or organs are subsequently implanted back into the patient.

In certain embodiments, FoxM1B protein, FoxM1B protein-encoding recombinant nucleic acid constructs or pharmaceutical compositions of compounds identified in a screening method of the invention can be delivered by implanting certain cells that have been genetically engineered, using methods such as those described herein, to express and secrete the polypeptide. Such cells may be animal or human cells, and may be autologous, heterologous, or xenogeneic, or may be immortalized. In order to decrease the chances of an immunological response, the cells may be encapsulated to avoid infiltration of surrounding tissues. Encapsulation materials are typically biocompatible, semi-permeable polymeric enclosures or membranes that allow the release of the protein product(s) but prevent the destruction of the cells by the patient's immune system or by other detrimental factors from the surrounding tissues.

Pharmaceutical compositions of the invention can be administered alone or in combination with other therapeutic agents, in particular, in combination with other cancer therapy agents. Such agents generally include radiation therapy or chemotherapy. Chemotherapy, for example, can involve treatment with one or more of the following agents: anthracyclines, taxol, tamoxifene, doxorubicin, 5-fluorouracil, and other drugs known to one skilled in the art.

FoxM1B polypeptide cell therapy, e.g., the implantation of cells producing FoxM1B polypeptides, is also contemplated. This embodiment of the invention involves implanting cells capable of synthesizing and secreting a biologically active form of FoxM1B polypeptide. Such FoxM1B polypeptide-producing cells can be cells that are natural producers of FoxM1B polypeptides or may be recombinant cells whose ability to produce FoxM1B polypeptides has been augmented by transformation with a gene encoding the desired FoxM1B polypeptide or with a gene augmenting the expression of FoxM1B polypeptide. Such a modification may be accomplished by means of a vector suitable for delivering the gene as well as promoting its expression and secretion. In order to minimize potential immunological reaction in patients being administered an FoxM1B polypeptide, as may occur with the administration of a polypeptide of a foreign species, it is preferred that natural cells producing FoxM1B polypeptide be of human origin, most preferably autologous to the individual in whom they are implanted, and produce human FoxM1B polypeptide. Likewise, it is preferred that the recombinant cells, most preferably cells autologous to the individual in whom they are implanted, that produce FoxM1B polypeptide be transformed with an expression vector containing a gene encoding a human FoxM1B polypeptide.

Implanted cells may be encapsulated to avoid infiltration of surrounding tissue. Human or non-human animal cells may be implanted in patients in biocompatible, semipermeable polymeric enclosures or membranes that permit release of FoxM1B polypeptide, but that prevent destruction of the cells by the patient's immune system or by other detrimental factors from surrounding tissue. Alternatively, autologous cells, i.e., the patient's own cells, transformed to produce FoxM1B polypeptides ex vivo, may be implanted directly into the patient without such encapsulation.

Techniques for the encapsulation of living cells are known in the art, and preparation of encapsulated cells and their implantation in patients may be routinely accomplished. For example, Baetge et al. (PCT Pub. No. WO 95/05452 and PCT/US94/09299) describe membrane capsules containing genetically engineered cells for effective delivery of biologically active molecules. The capsules are biocompatible and are easily retrievable. The capsules encapsulate cells transfected with recombinant DNA molecules comprising DNA sequences encoding biologically active molecules operatively linked to promoters that are not subject to down-regulation in vivo upon implantation into a mammalian host. Such devices provide for the delivery of the molecules from living cells to specific sites within a recipient. See U.S. Pat. Nos. 4,892,538; 5,011,472; and 5,106,627. A system for encapsulating living cells is described in PCT Pub. No. WO 91/10425 (Aebischer et al.). See also, PCT Pub. No. WO 91/10470 (Aebischer et al.); Winn et al., 1991, Exper. Neurol. 113:322-29; Aebischer et al., 1991, Exper. Neurol. 111:269-75; and Tresco et al., 1992, ASAIO 38:17-23 for art-recognized systems for encapsulating living cells.

In vivo, ex vivo and in vitro gene delivery of FoxM1B polypeptides is also provided herein. One example of a gene therapy technique is to use a full-length or less than full-length FoxM1B gene (either genomic DNA, cDNA, and/or synthetic DNA) encoding a FoxM1B polypeptide that can be operatively linked to a constitutive or inducible promoter to form a “gene therapy DNA construct.” The promoter may be homologous or heterologous to the endogenous FoxM1B gene, provided that it is active in the cell or tissue type into which the construct is inserted. Other components of the gene therapy DNA construct may optionally include DNA molecules designed for site-specific integration (e.g., endogenous sequences useful for homologous recombination), tissue-specific promoters, enhancers or silencers, DNA molecules capable of providing a selective advantage over the parent cell, DNA molecules useful as labels to identify transformed cells, negative selection systems, cell specific binding agents (for example, for cell targeting), cell-specific internalization factors, transcription factors enhancing expression from a vector, and factors enabling vector production.

A gene therapy DNA construct can then be introduced into cells (either ex vivo or in vivo) using viral or non-viral vectors. One means for introducing the gene therapy DNA construct is by means of viral vectors as described herein. Certain vectors, such as retroviral vectors, will deliver the DNA construct to the chromosomal DNA of the cells, and the gene can integrate into the chromosomal DNA. Other vectors will function as episomes, and the gene therapy DNA construct will remain unintegrated, for example, in the cell cytoplasm.

In yet other embodiments, regulatory elements can be included for controlled expression of a full-length or less than full-length FoxM1B gene in a target cell. Such elements are activated in response to an appropriate effector. In this way, a therapeutic polypeptide can be expressed when desired. One conventional control means involves the use of small molecule dimerizers or rapalogs to dimerize chimeric proteins which contain a small molecule-binding domain and a domain capable of initiating a biological process, such as a DNA-binding protein or transcriptional activation protein (see PCT Pub. Nos. WO 96/41865, WO 97/31898, and WO 97/31899). The dimerization of the proteins can be used to initiate transcription of the transgene.

In vivo gene therapy may be accomplished by introducing the gene encoding FoxM1B polypeptide into cells via local delivery of a FoxM1B nucleic acid molecule, by direct injection or by other appropriate viral or non-viral delivery vectors. (Hefti, 1994, Neurobiology 25:1418-35.) For example, a nucleic acid molecule encoding a FoxM1B polypeptide may be contained in an adeno-associated virus (AAV) vector for delivery to the targeted cells (see, e.g., Johnson, PCT Pub. No. WO 95/34670; PCT App. No. PCT/US95/07178). The recombinant AAV genome used according to the teachings of the invention typically contains AAV inverted terminal repeats flanking a DNA sequence encoding a FoxM1B polypeptide operatively linked to functional promoter and polyadenylation sequences.

Alternative suitable viral vectors include, but are not limited to, retrovirus, adenovirus, herpes simplex virus, lentivirus, hepatitis virus, parvovirus, papovavirus, poxvirus, alphavirus, coronavirus, rhabdovirus, paramyxovirus, and papilloma virus vectors. U.S. Pat. No. 5,672,344 describes an in vivo viral-mediated gene transfer system involving a recombinant neurotrophic HSV-1 vector. U.S. Pat. No. 5,399,346 provides examples of a process for providing a patient with a therapeutic protein by the delivery of human cells that have been treated in vitro to insert a DNA segment encoding a therapeutic protein. Additional methods and materials for the practice of gene therapy techniques are described in U.S. Pat. No. 5,631,236 (involving adenoviral vectors), U.S. Pat. No. 5,672,510 (involving retroviral vectors), and U.S. Pat. No. 5,635,399 (involving retroviral vectors expressing cytokines).

Nonviral delivery methods include, but are not limited to, liposome-mediated transfer, naked DNA delivery (e.g., by direct injection), receptor-mediated transfer (ligand-DNA complex), electroporation, calcium phosphate precipitation, and microparticle bombardment (e.g., gene gun). Gene therapy materials and methods may also include inducible promoters, tissue-specific enhancer-promoters, DNA sequences designed for site-specific integration, DNA sequences capable of providing a selective advantage over the parent cell, labels to identify transformed cells, negative selection systems and expression control systems (safety measures), cell-specific binding agents (for cell targeting), cell-specific internalization factors, and transcription factors to enhance expression by a vector as well as methods of vector manufacture. Such additional methods and materials for the practice of gene therapy techniques are described in U.S. Pat. No. 4,970,154 (involving electroporation techniques), U.S. Pat. No. 5,679,559 (describing a lipoprotein-containing system for gene delivery), U.S. Pat. No. 5,676,954 (involving liposome carriers), U.S. Pat. No. 5,593,875 (describing methods for calcium phosphate transfection), and U.S. Pat. No. 4,945,050 (describing a process wherein biologically active particles are propelled at cells at a speed whereby the particles penetrate the surface of the cells and become incorporated into the interior of the cells), and PCT Pub. No. WO 96/40958 (involving nuclear ligands).

It is also contemplated that FoxM1B gene therapy or cell therapy can further include the delivery of one or more additional polypeptide(s) in the same or a different cell(s). Such cells may be separately introduced into the patient, or the cells may be contained in a single implantable device, such as the encapsulating membrane described above, or the cells may be separately modified by means of viral vectors.

Another means of increasing endogenous FoxM1B polypeptide expression in a cell via gene therapy is to insert one or more enhancer elements into the FoxM1B polypeptide promoter, where the enhancer elements can serve to increase transcriptional activity of a full-length or less than full-length FoxM1B gene. The enhancer elements used are selected based on the tissue in which one desires to activate the gene-enhancer elements that are known to confer promoter activation in that tissue are preferred. For example, if a gene encoding a FoxM1B polypeptide is to be “turned on” in T-cells, the ick promoter enhancer element may be used. Here, the functional portion of the transcriptional element to be added may be inserted into a fragment of DNA containing the FoxM1B polypeptide promoter (and optionally, inserted into a vector and/or 5′ and/or 3′ flanking sequences) using standard cloning techniques. This construct, known as a “homologous recombination construct,” can then be introduced into the desired cells either ex vivo or in vivo.

The following Examples are provided for the purposes of illustration and are not intended to limit the scope of the present invention. The present invention is not to be limited in scope by the exemplified embodiments, which are intended as illustrations of individual aspects of the invention. Indeed, various modifications of the invention in addition to those shown and described herein will become apparent to those skilled in the art from the foregoing description and accompanying drawings. Such modifications are intended to fall within the scope of the appended claims.

### EXAMPLES

### Example 1

Effects of Increased FoxM1B Expression on DNA Replication and Mitosis in Regenerating Liver of Aged Transgenic Mice

Transgenic CD-1 mice were generated using the −3 kb transthyretin (TTR) promoter to constitutively express the FoxM1B transgene (SEQ ID NO: 1 as shown in FIG. 1) in hepatocytes as described (Ye et al., 1999, Mol. Cell Biol., 19: 8570-8580). The human FoxM1B cDNA used for the FoxM1B transgene cDNA is comprised of a deletion of the terminal 972 nucleotides at the 3′ end of the FoxM1B cDNA leaving a 2737 nucleotide FoxM1B cDNA fragment transgene (SEQ ID NO: 1 as shown in FIG. 1) that encodes the entire FoxM1B protein. Expression of FoxM1B increases during cellular proliferation through stabilization of the FoxM1B mRNA. Unlike the endogenous FoxM1B mRNA, when the 2737 nucleotide FoxM1B cDNA fragment transgene (SEQ ID NO: 1 as shown in FIG. 1) is expressed as RNA in non-dividing cells it is stable in non-dividing cells and will accumulate in non-dividing cells (Ye et al., 1999, Mol. Cell Biol., 19: 8570-8580). The deletion of the terminal 972 nucleotides at the 3′ end of the FoxM1B cDNA therefore contains sequences that mediate RNA degradation of FoxM1B mRNA in non-dividing cells. The FoxM1B transgene cDNA (SEQ ID NO: 1 as shown in FIG. 1) therefore provides advantages in the ability to deliver stabilized FoxM1B mRNA to non-dividing cells and expression of FoxM1B protein to non-dividing cells. Expression of the FoxM1B transgene protein in non-dividing cells will remain cytoplasmic unless the hepatocyte or other cell type is stimulated to undergo cell division (Ye et al., 1999, Mol. Cell Biol., 19: 8570-8580). However, the FoxM1B transgene mRNA primes the hepatocytes for DNA replication because FoxM1B transgene mRNA and protein is available to facilitate entry into the cell cycle once the cell receives proliferative signaling from growth factors.

Twelve-month old wild type CD-1 (WT) and TTR-FoxM1B (TG) mice were anesthetized with methoxyflurane (Metofane; Schering-Plough Animal Health Corp., Union, N.J.) and the left lateral, left median, and right median lobes of the liver were removed following midventral laparotomy to induce liver regeneration (Higgins et al., 1931, Arch. Pathol. 12:186-202). Removal of the gallbladder, located between the left and right median lobes was carefully avoided. Following surgery, animals were given one subcutaneous injection of ampicillin (50 μg/g body weight) in saline. Two hours prior to harvesting the remnant liver, animals were injected intraperitoneally with 10 mg/mL of 5-bromo-2′-deoxyuridine (BrdU; 50 μg/g body weight) in phosphate-buffered saline (PBS). Two mice were sacrificed by CO₂ asphyxiation at 24, 32, 36, 40, 44, and 48 hours after partial hepatectomy (PHx) surgery and their livers were removed. The dissected livers were divided into three portions: one for paraffin embedding, one for total RNA isolation, and one for total protein isolation.

Liver portions for paraffin embedding were fixed in 4% paraformaldehyde overnight and embedded in paraffin. Tissues were cut into 5 μm sections with a microtome and fixed onto slides. Sections were dewaxed with xylenes, rehydrated with decreasing graded ethanol washes, and placed in PBS with 0.25% Triton X-100 (PBT). A microwave antigen-retrieval method was used to enhance antigenic reactivity of the antibodies as previously described (Zhou et al., 1996, J Histochem. Cytochem. 44:1183-1193). Sections were immunohistochemically stained with anti-BrdU monoclonal antibodies according to the manufacturer's instructions (Boehringer Mannheim). The number of BrdU positive nuclei per 1000 hepatocytes was counted and the mean BrdU positive cells and standard deviation (SD) were calculated using two regenerating liver samples from each time point. Regenerating livers from 2 month old (young) CD-1 mice were examined and included as a comparison. The 2 month old livers display an S-phase peak at 40 hours after PHx (FIG. 2). A much smaller 40-hour S-phase peak was observed in the regenerating livers from 12 month old WT mice (FIG. 2). The regenerating livers of 12 month old TG mice exhibited a sharp S-phase peak at 40 hours similar to that observed in the 2 month old livers (FIG. 2). Immunohistochemical staining with anti-BrdU antibodies shows the increase in BrdU incorporation in the TG livers compared with the WT livers at 40 hours. In addition, at 48 hours post PHx, the regenerating hepatocytes of the old WT mice displayed fewer mitotic figures compared with those of the TG mice (FIG. 3).

These studies demonstrated that increased hepatocyte expression of FoxM1B in regenerating livers of old-aged transgenic mice stimulated hepatocyte DNA replication and mitosis to levels found in young regenerating mouse liver.

### Example 2

The Effects of PHx on the Levels of FoxM1B mRNA and Protein Expression in Young and Old WT Mice and Old TG Mice

Total RNA from regenerating livers of wild type (WT) and transgenic (TG) mice was extracted 24, 32, 36, 40, and 44 hours post partial hepatectomy (PHx) by an acid guanidium thiocyanate-phenol-chloroform extraction method with RNA-STAT-60 (Tel-Test “B” Inc., Friendswood, Tex.). Antisense RNase protection probes for the human and mouse FoxM1B transgene and for mouse cyclophilin were generated as described (Ye et al., 1997, Mol. Cell Biol.17:1626-1641; Wang et al., 2001, Hepatology 33:1404-1414). RNase protection assays were performed by hybridizing 20 to 40 μg of total liver RNA with {³²P} UTP-labeled probes followed by digestion with RNase One, electrophoresis, and autoradiography as described previously (Ye et al., 1997, Mol. Cell Biol. 17:1626-1641; Wang et al., 2001, Hepatology 33:1404-1414; Rausa et al., 2000, Mol. Cell Biol. 20:8264-8282). X-ray films were scanned and the BioMax iD program (Eastman Kodak Co) was used to quantify expression levels, which were normalized to cyclophilin RNA levels. FoxM1B mRNA levels were induced at 40 hours, consistent with the S-phase peak, in the regenerating liver from 2 month old WT mice (FIG. 4A, FIG. 2). Likewise, the S-phase peak observed in old TG mice at 40 hours post PHx was accompanied by elevated FoxM1B mRNA (FIG. 4B). Induction of FoxM1B mRNA at 40 hours was diminished in 12 month old WT mice compared with the young mice (FIGS. 4A and B).

Total protein extracts from regenerating livers of 12 month old TG and WT mice at 24, 32, 36, 40, and 44 hours after PHx were isolated as described (Rausa et al., 2000, Mol. Cell Biol. 20: 8264-8282). Western blot analysis was done by separating 50 μg of total liver protein by SDS-PAGE, transferring to Protran membrane (Schleicher & Schuell, Keene, N.H.), incubating with HFH-11 (FoxM1B) antibody (Ye et al., 1997, Mol. Cell Biol. 17: 1626-1641; Ye et al., 1999, Mol. Cell Biol. 19: 8570-8580), and amplifying the signal with biotin conjugated anti-rabbit IgG (BioRad, Hercules, Calif.). Signal was detected with enhanced chemiluminesence (ECL, Amersham Pharmacia Biotech, Piscataway, N.J.). Elevated protein levels of FoxM1B were associated with increased BrdU incorporation and FoxM1B mRNA expression at 40 hours after PHx (FIGS. 3, 4C, and 5). No increase in FoxM1B protein expression was observed in regenerating hepatocytes of old-aged WT mice (FIG. 5).

These studies demonstrated that increased FoxM1B mRNA and protein levels in transgenic mice is associated with increased hepatocyte proliferation in regenerating liver of old-aged transgenic mice.

### Example 3

Altered Expression of Genes Involved in S-Phase and M-Phase Progression in Response to Increased Expression of FoxM1B in Regenerating Livers

RNase protection assays were performed for a number of genes associated with cell cycle progression. Probes for Cyclin D1, Cyclin D3, Cyclin E, Cyclin A1, Cyclin A2, Cyclin B1, Cyclin B2, and Cyclin F were purchased from Pharmingen (San Diego, Calif.) and probes for Cdc25B and p55Cdc were purchased from Clontech. RNase protection assays were performed for Cyclin genes using procedures described by the manufacturer and for other genes as described above on 20-40 μg of total liver RNA isolated from WT and TG mice 24, 32, 36, 40, and 44 hours after PHx.

The results of these assays are shown in FIG. 6. Expression of the Cyclin D1 gene, which promotes S-phase, was elevated in the aged TG mice at 36 to 40 hours post PHx, just before the initiation of hepatocyte DNA replication (FIG. 6). Expression levels of Cyclin E were also increased at 40 hours post PHx in old TG mice (FIG. 6). The induction of Cyclin D1 and Cyclin E in the regenerating livers of TG mice is associated with increased expression of FoxM1B. Cyclin D1 and Cyclin E expression was decreased during the G1/S transition of the cell cycle of regenerating livers of old WT mice (FIG. 6). In addition, elevated FoxM1B levels led to increased expression of Cyclin A2 in these livers (FIG. 6). The data show that restoring FoxM1B expression in regenerating liver of old mice stimulates the induction of Cyclin D1, Cyclin E, and Cyclin A2, which facilitate hepatocyte entry and progression through S-phase.

During the peak of hepatocyte DNA replication, a significant induction of Cyclin B1 and Cyclin B2 was observed only in the regenerating liver from old TG mice (FIG. 6). Cyclin F levels also were increased significantly in the regenerating liver of 12 month old TG mice at this time point in the experiments (FIG. 6). Greater activation of Cdc25B mRNA was observed between 40 and 44 hours post PHx in the liver of TG animals than in the liver of WT animals (FIG. 6). In addition, only the liver of TG animals displayed induced expression of p55Cdc after PHx (FIG. 6). Cyclin B1 and Cyclin B2 mediate cell cycle progression from the G2 phase into mitosis (Zachariae et al., 1999, Genes Dev. 13: 2039-2058). Cyclin F is essential for M-phase progression because it facilitates nuclear translocation of the Cyclin B complexes (Kong et al., 2000, EMBO J. 19: 1378-1388). M-phase progression is also mediated by Cdc25B, which activates the mitotic kinase cdk1/cyclin B (Sebastian et al., 1993, Proc. Natl. Acad. Sci. USA 90: 3521-3524; Trembley et al., 1996, Cell Growth Differ. 7: 903-916; Nilsson et al., 2000, Prog. Cell Cycle Res. 4: 107-114). Degradation of Cyclin proteins, a process necessary for completion of mitosis, is regulated by p55Cdc (Zachariae et al., 1999, Genes Dev. 13: 2039-2058).

These results demonstrated that increased expression of FoxM1B in old TG mice induces M-phase promoting genes including Cyclin B1, Cyclin B2, Cyclin F, Cdc25B, and p55Cdc.

### Example 4

p21 and p53 Expression in the Liver of Old FoxM1B Transgenic Mice After Partial Hepatectomy

Twenty to forty micrograms of total liver RNA was isolated from old TG and WT mice 24, 32, 36, and 40 hours after PHx. An RNase protection probe for p21 was received as a gift from Dr. Guy Adami (University of Illinois at Chicago). As above, approximately 2×10⁵ cpm of each probe was hybridized at 45° C. or 55° C. to 20 μg of total RNA in a solution containing 20 mM PIPES (pH6.4), 400 mM NaCl, 1 mM EDTA and 80% formamide overnight. After hybridization, samples were digested for 1 hr at 37° C. by using 10 units per sample of RNase One enzyme according to the manufacturer's protocol (Promega, Madison, Wis.). The RNase One protected fragments were electrophoresed on an 8% polyacrylamide-8M urea gel, followed by autoradiography. Quantitation of expression levels was determined with scanned X-ray films by using the BioMax 1D program (Eastman Kodak, Rochester, N.Y.). The cyclophilin hybridization signal was used for a normalization control between different liver RNA samples. p21 mRNA levels were decreased during the G1/S transition of the cell cycle in the old TG animals (FIG. 7, 32 to 40 hours post PHx).

Paraffin embedded tissue samples from regenerating livers of 12 month old WT and TG mice dissected 24, 32, and 40 hours post PHx were sectioned with a microtome and prepared for immunohistochemical staining as described above. Sections were incubated with anti-p21 antibodies (Oncogene Science, Cambridge, Mass.) or anti-FoxM1B antibodies and detected using the ABC kit and DAB peroxidase substrate according to manufacturer's instructions (Vector Laboratories, Burlingame, Calif.). The number of p21 positive and FoxM1B positive hepatocytes per 1000 nuclei for each mouse liver was determined, and data from two mice for each time point were used to calculate the mean±standard deviation (SD) using the Analysis ToolPak in Macintosh Microsoft Excel 98. p21 protein levels in the nuclei of regenerating liver of old TG mice were reduced compared with levels observed in the WT liver at 32 hours after PHx (FIG. 8). However, at 36 hours after PHx, p21 nuclear protein levels in liver of TG mice were similar to those in WT liver (FIG. 8), which is consistent with the role of p21 in assembling the Cyclin D/cdk4/6 complex necessary for progression into S-phase (Cheng, et al., 1999, EmboJ 18:1571-1583).

The ability of increased FoxM1B expression to mediate diminished p53 protein levels in regenerating hepatocytes of old-aged TTR-FoxM1B TG mice was also examined. Prior to hepatocyte DNA replication (24 to 36 hours post PHx), Western blot analysis revealed a 50-70% reduction in p53 protein levels in regenerating livers of old-aged TTR-FoxM1B TG mice compared to old-aged WT mice (FIGS. 9A-C). Coincident with the reduction of p53 protein levels, a 50% reduction in p21^{Cip1} protein expression prior to S-phase in regenerating livers of old-aged TTR-FoxM1B TG mice was observed.

These liver regeneration studies indicated that maintaining FoxM1B expression levels in these cells caused diminished expression of p53 and p21^{Cip1} proteins during the G1 to S-phase transition in old-aged TTR FoxM1B TG mice, which is consistent with preventing reduced proliferating associated with an aging phenotype.

### Example 5

The Effects of Carbon Tetrachloride Induced Liver Injury on Localization of FoxM1B and Hepatocyte DNA Replication in FoxM1B Transgenic Mice

Wild type or FoxM1B transgenic male CD-1 mice (8-10 weeks of age) were given a single intraperitoneal (IP) injection of a 10% solution of carbon tetrachloride (10 μl CCl₄/g body weight; Sigma-Aldrich, St. Louis, Mo.) dissolved in light mineral oil, as described in Serfas et al., 1997, Cell Growth Differ. 8:951-961. Mice were subjected to an IP injection of 10 mg/mL solution of 5-bromo-2′-deoxyuridine (BrdU; 50 μg/g body weight) in phosphate buffered saline (PBS) two hours prior to harvesting the liver as described previously (Ye et al., 1999, Mol. Cell Biol. 19: 8570-8580). Mice were sacrificed by CO₂ asphyxiation at 16, 20, 24, 28, 32, 34, 36, 40, 44, and 48 hour intervals following CCl₄ administration. A portion of liver tissue was used to prepare total RNA and the rest of the liver was paraffin embedded as described previously (Id.). To determine the statistical significance of any observed differences between transgenic and wild type mice four mice were sacrificed at each time point.

Nuclear localization of FoxM1B protein requires proliferative signaling (Id). Therefore, an affinity-purified FoxM1B antibody was used as above for immunohistochemical staining of mouse liver sections at the various time points following CC14 liver injury. Regenerating WT hepatocytes displayed FoxM1B nuclear staining between 32 to 36 hours following CCl₄ liver injury (FIGS. 10A-B) and reached maximum staining by the 40-hour time point (FIG. 10C). In contrast, nuclear FoxM1B protein staining was found in regenerating TG hepatocytes at the earliest time point examined (20 hours after CCl₄ injury) and persisted throughout the liver regeneration process (FIGS. 10D-F).

The timing of hepatocyte entry into S-phase and DNA synthesis in CC14 regenerating liver was examined by immunohistochemical staining of BrdU incorporation into DNA as described above. In WT livers, a few BrdU positive staining hepatocytes were detected at 36 hours after CCl₄ injury, while hepatocyte DNA replication reached a maximum by 40 hours and displayed a broad persistent S-phase peak (FIG. 11). In contrast, TG hepatocytes showed detectable BrdU incorporation at 32 hours after CC14 injury, while hepatocyte replication was significantly increased by 34 hours and became maximal by 36 hours (FIG. 11).

These studies show earlier nuclear expression of the FoxM1B transgene protein results in a six-hour acceleration in the onset of hepatocyte DNA replication following liver injury induced by CCl₄.

### Example 6

The Effects of Carbon Tetrachloride Induced Liver Injury on p21 Levels in FoxM1B Transgenic Mice

To determine whether earlier transgenic hepatocyte replication correlates with diminished p21 protein expression, livers of WT and TG mice were removed 16, 20, 24, 28, 32, 36, and 40 hours after CCl₄ induced liver injury and examined by immunohistochemical staining as described above with anti-p21 antibodies. The number of p21 staining periportal hepatocytes present in regenerating TG hepatocytes was significantly decreased between 16 and 36 hours post CCl₄ liver injury compared with regenerating WT hepatocytes (FIG. 12A). The difference in hepatocyte expression of p21 protein was greatest at 36 hours following CCl₄ administration (FIG. 12A), corresponding to the time of maximum TG hepatocyte DNA replication and barely detectable WT hepatocyte replication (FIG. 11). The p21 expression pattern was the same at 40 hours post CCl₄ liver injury when both WT and TG hepatocytes show abundant BrdU incorporation.

The level of p21 mRNA expression was also examined in CC14 regenerating livers of TG mice and WT mice. RNase protection assays were performed as described in duplicate. Hepatic p21 mRNA was normalized and is presented graphically, demonstrating that regenerating WT hepatic expression of p21 remained constant throughout the time points considered (FIG. 12B). A significant reduction in TG hepatic levels of p21 mRNA was observed between 28 and 32 hours following CCl₄ liver injury (FIG. 12B), which is consistent with early hepatocyte entry into S-phase as seen in FIG. 11.

These studies demonstrated that diminished expression of p21, which is inhibitory to DNA replication, mediates accelerated hepatocyte proliferation during liver regeneration.

### Example 7

Differential Expression of Proliferation-Specific Genes in Regenerating Livers of Transgenic and Wild Type Mice Following CCl₄ Liver Injury

As described above, RNase protection assays were performed with Cyclin genes using RNA protection probes and a kit made by Pharmingen (San Diego, Calif.) following procedures recommended by the manufacturer. The ribosomal large subunit protein L32 and glyceraldehyde-3-phosphate dehydrogenase GAPDH signals were used to normalize Cyclin expression at the different time points during CCl₄ liver regeneration. Antisense RNA probes for mouse Cdc25a and Cdc25b were generated from Atlas cDNA plasmids purchased from Clontech (Paolo Alto, Calif.).

RNase protection assays were performed in duplicate to examine the temporal expression patterns of the Cyclin genes in CC14 regenerating TG and WT livers. Compared with regenerating WT liver, regenerating TG liver displayed early increases in expression of S-phase promoting Cyclin D1 and E genes between 24 to 36 hours after CC14 injury, corresponding to the G1/S transition of the cell cycle. The CCl₄ regenerating TG livers displayed a more significant peak in CyclinD1 expression compared with the regenerating WT livers (FIG. 13A), suggesting that premature FoxM1B expression can induce Cyclin D1 expression and accelerate hepatocyte entry into S-phase.

The induction peaks of Cyclin D1 and Cyclin E expression following CCl₄ liver injury in TG mice differ from those observed in the PHx liver regeneration model. Regenerating TG liver displayed a persistent increase in hepatic Cyclin D1 levels from 28 hours post PHx until initiation of DNA replication, and no changes were found in the induction of Cyclin E expression (Ye et al., 1999, Mol. Cell Biol. 19: 8570-8580). Regenerating livers induced by PHx or CCl₄ both exhibit early activation of Cyclin A2 expression (FIG. 13D, Id.). Cyclin A2 complexes with CDK2 and is essential for S-phase progression by mediating E2F phosphorylation, which inactivates its DNA binding activity (Dynlacht et al., 1994, Genes Dev. 8: 1772-1786; Xu et al., 1994, Mol. Cell Biol. 14: 8420-8431).

As observed in previous PHx regeneration studies, which demonstrated an 8 hour acceleration in entry into mitosis coinciding with early expression of Cyclin B1 and B2 genes (Ye et al., 1999, Mol. Cell Biol. 19: 8570-8580), CCl₄-regenerating TG liver displayed early hepatic expression of Cyclin B1 and B2 genes (FIG. 13C). Also, both liver regeneration models displayed early induction of Cyclin F levels at the peak of hepatocyte DNA replication (FIG. 13E). Cyclin F may mediate nuclear localization of the Cyclin B proteins and entry into mitosis (Kong et al., 2000, EMBO J. 19: 1378-1388). These results suggested that early Cyclin F expression may elicit earlier TG hepatocyte entry into M-phase by facilitating Cyclin B nuclear localization. In addition, analysis of these liver regeneration models studies suggest that FoxM1B activates distinct S-phase promoting pathways following CCl₄ liver injury, but they displayed activation of similar Cyclin genes for accelerated entry into M-phase.

RNase protection assays also demonstrated that high levels of Cdc25a mRNA are maintained between 24 and 40 hours after CCl₄ injury in regenerating TG liver, while Cdc25a expression in regenerating WT liver decreases sharply after the 28 hour time point (FIGS. 13F and G). Cdc25a expression was sustained through the peak of TG hepatocyte DNA replication allowing for progression into S-phase through activation of the CyclinD1/CDK4 complex. At the peak of TG hepatocyte replication, an increase in Cdc25b (cdc25M2) phosphatase levels was observed (FIG. 13G). Early activation of Cdc25b mRNA levels was seen in regenerating TG liver at 36 hours post CC14 injury, whereas its expression did not increase in WT regenerating liver until the 40 hour time point (FIG. 13G). Cdc25b regulated M-phase progression by activating the mitotic kinase Cdk1/cyclin B via dephosphorylation (Nilsson et al., 2000, Prog. Cell Cycle Res. 4: 107-114; Sebastian et al., 1993, Proc. Natl. Acad. Sci. USA. 90: 3521-3524; Trembley et al., 1996, Cell Growth Differ. 7: 903-916). Early expression of Cdc25b promotes entry into mitosis by activating cdk1-cyclinB kinase activity, which is required to initiate and execute mitosis (division of duplicated chromosomes to daughter cells).

These results demonstrated that premature expression of FoxM1B activated earlier expression of Cdc25B phosphatase, which accelerated entry into M-phase.

### Example 8

Expression of FoxM1B by Adenoviral Delivery of the FoxM1B Gene to Livers of Mice

Twelve month old Balb/c mice were obtained from the National Institute of Aging and were infected by tail vein injection with either adenovirus vectors expressing FoxM1B (AdFoxM1B) or adenovirus vector as a control (AdEmpty) (1×10¹¹ purified adenovirus particles). The adenovirus-expressing FoxM1B (AdFoxM1B) was generated by subcloning the 2.7 kB EcoRI-HindIII fragment of the human FoxM1B cDNA into the adenovirus shuttle vector pGEMCMV NEW (gift from J. R. Nevins, Duke University). Greater than 95% of the adenovirus infects the liver after tail vein injection with minimal infection of other organs. Adenovirus is efficiently delivered to most cells throughout the liver parenchyma. Mouse tail vein injection of AdFoxM1B effectively increases in vivo hepatic expression of FoxM1B.

Two days after tail vein injection, infected mice were subjected to partial hepatectomy (PHx) operation as described above. PHx operation was performed two days after adenovirus infection to avoid the initial acute phase response to viral infection, which is completed within the first 36 hours following adenovirus infection. An intraperitoneal (IP) injection of a phosphate buffered saline (PBS) solution containing 10 mg/mL BrdU (Sigma; 50 μg/g body weight) was administered two hours prior to harvesting the remnant regenerating liver, samples of which were harvested at different intervals between 24 and 48 hours following surgery as previously described (Ye et al., 1999, Mol. Cell Biol. 19:8570-8580).

The liver tissue was used to prepare total RNA or paraffin embedded for immunohistochemical staining of BrdU incorporation into DNA to monitor hepatocyte DNA replication as described previously. RNase protection assays were performed with the FoxM1B RNase protection probe as described above, and demonstrated that AdFoxM1B infection elicited a large increase in FoxM1B mRNA (FIG. 14A). For comparison, RNase protection assays were performed on liver RNA isolated from regenerating livers of 2 month-old (young) mice. Significant increases in FoxM1B expression were observed in these samples between 36 and 44 hours following PHx; high expression levels were detected and were sustained for the duration of the liver regeneration experiment (FIG. 14A). In contrast, RNase protection assays with RNA from regenerating livers of old-aged mice that were AdEmpty infected displayed only minimal increase in FoxM1B mRNA at 24 hours post PHx with a second increase at 40 hours (FIG. 14A). Also, a small increase in FoxM1B expression was observed throughout the time points examined from uninfected regenerating liver of old mice (FIG. 14A).

Paraffin embedded liver tissues were subjected to immunostaining with anti-BrdU antibodies and the expression pattern of the FoxM1B protein was examined by immunohistochemistry using FoxM1B protein as described above. The adenovirus-mediated increase in FoxM1B expression stimulated an earlier peak in hepatocyte DNA replication at 32 hours post PHx (FIG. 14B), which normally occurs at 40 hours post PHx in young Balb/c mice. Consistent with the role of FoxM1B in mediating progression into S-phase, regenerating liver infected with AdEmpty or mock infected lacked significant increase in hepatocyte DNA replication (FIG. 14B). Hepatocyte mitotic figures were examined and are represented graphically in FIG. 14C. Adenovirus mediated increase in FoxM1B expression stimulated hepatocyte mitosis between 36 to 44 hours post PHx compared to regenerating livers of old mice infected with either control adenovirus or uninfected (FIG. 14C). Immunohistochemical staining of regenerating liver from old mice infected with AdEmpty exhibited undetectable nuclear protein levels of FoxM1B following PHx (FIG. 15, left panel). Nuclear FoxM1B protein expression was observed in all time points between 24 and 36 hours (FIG. 15, right panel).

These results showed that adenovirus-mediated increase in hepatic levels of FoxM1B restored hepatocyte progression into S-phase and mitosis at a rate similar to that found in young regenerating liver.

### Example 9

Expression of Cell Cycle Regulatory Genes is Restored in Regenerating Livers of Old-Aged Mice Expressing AdFoxM1B

Expression of S-Phase and M-Phase Promoting Genes

RNase protection assays were performed in triplicate with cell cycle regulatory gene probes, and regenerating liver RNA was isolated between 24 and 44 hours following PHx (FIGS. 16A-D). In 2 month-old (young) regenerating mouse liver, increased S-phase levels of Cyclin A2, Cyclin B1 and Cyclin B2 mRNA occurred at 40 hours post PHx (FIGS. 16E-G). Regenerating liver of AdFoxM1B infected old-aged mice displayed increased S-phase levels of Cyclin A2, Cyclin B1 and Cyclin B2 mRNA at 32 hours post PHx (FIGS. 16E-G) compared to either MI or AdEmpty infected old-aged mouse controls (FIGS. 16E-G). Furthermore, expression levels of these genes diminished during mitosis, which occurred at 36 hours following PHx (FIGS. 16E-G). To identify cell cycle regulatory genes whose expression is restored in regenerating liver of old mice infected with AdFoxM1B, RNase protection assays were performed as described in duplicate with probes against various Cyclin genes with RNA isolated from regenerating liver of old-aged Balb/c mice infected with AdEmpty or AdFoxM1B as above.

Taken together, these data indicated that restoring FoxM1B expression in regenerating liver of old mice stimulated induction of S-phase promoting Cyclin A2 and M-phase promoting Cyclin B, which served to facilitate hepatocyte proliferation.

### Example 10

AdFoxM1B Infection of Old-Aged Mice Causes Diminished p27^{kip1} Protein Levels and Increased Cdk2 Kinase Activity

The p27^{Kip1} (p27) protein associates with Cdk2 and inhibits kinase activity of the Cyclin E-Cdk2 and Cyclin A2-Cdk2 complexes (Sherr and Roberts, 1999, Genes Dev. 13:1501-1512). Cell growth, specifically progression of the S-phase of the cell cycle, requires Cyclin-Cdk2 protein phosphorylation of the Retinoblastoma (RB) protein, which causes dissociation of RB and activates the E2F transcription factor (Harbour and Dean, 2000, Genes Dev. 14:2393-2409). To determine if FoxM1B could influence p27 expression, mice expressing AdFoxM1B were subjected to partial hepatectomy (PHx) experiments as described above.

Total protein extracts were isolated from regenerating livers of 12-month old AdFoxM1B mice, 2-month old mock infected (MI) mice, 12-month old MI mice, and 12-month old AdEmpty (i.e., infected with adenovirus vector without the FoxM1B gene) infected mice. Fifty micrograms of total protein from each sample were separated on SDS-PAGE and transferred to Protran membranes (Schleicher and Schuell, Keene, N.H.). The membrane was stained with anti-p27 antibodies (Cell Signaling, Berkeley, Calif.) using conventional Western blotting techniques (Sambrook et al., Id.). The signal was amplified by biotin-conjugated anti-rabbit IgG (Bio-Rad, Hercules, Calif.) and detected with Enhanced Chemiluminescence Plus (ECL-plus, Amersham Pharmacia Biotech, Piscataway, N.J.) according to the manufacturer's instructions. The results, shown in FIG. 17, demonstrated that reduced hepatic levels of p27 are found in young MI mice and old aged AdFoxM1B mice following PHx.

Total RNA was prepared using RNA-STAT-60 (Tel-Test “B” Inc., Friendswood, Tex.) from liver tissues from 12-month old AdFoxM1B mice, 2-month old mock infected (MI) mice, 12-month old MI mice, and 12-month old AdEmpty-infected mice after PHx. A 600 nucleotide mouse p27 cDNA was digested with AvaII to generate a 200 nucleotide antisense RNA probe. RNA samples from the livers were hybridized with the {α-³²P} UTP labeled antisense probe and separated on an RNase protection assay gel. The gel was exposed to a phosphorimaging screen for 1 to 2 days and scanned with a Storm 860 phophorimager and quantitated using the ImageQuant program. Expression levels were normalized to cyclophilin mRNA levels. These RNase protection assays demonstrated that AdFoxM1B had no effect on the p27 mRNA levels (shown in FIGS. 17E-F).

Liver samples taken from 12-month old AdFoxM1B mice, 2-month old mock infected (MI) mice, 12-month old MI mice, and 12-month old AdEmpty-infected mice after PHx were prepared for immunohistochemical analysis. Using methods described herein and a 15-minute proteinase K antigen retrieval step (20 μg/mL Proteinase K, Invitrogen), the tissue samples were stained with anti-p27 antibodies (Cell Signaling). Livers from MI and AdEmpty infected old-aged mice displayed abundant p27 nuclear staining prior to S-phase (FIG. 18). AdFoxM1B infected old-aged mice displayed only perinuclear hepatocyte staining of p27 protein after PHx (FIG. 18). Although the MI and AdEmpty infected old-aged mice displayed perinuclear staining of p27 protein at 40 hours after PHx (FIGS. 18C and F), the delayed change in p27 cellular localization was unable to facilitate S-phase progression. These studies suggest that FoxM1B mediates S-phase progression in old-aged mice by diminishing nuclear expression of p27 protein.

To determine whether diminished p27 protein levels were associated with increased Cdk2 kinase activity, regenerating liver protein extracts were prepared from AdEmpty or AdFoxM1B infected old-aged mice and were immunoprecipitated with anti-Cdk2 antibodies. The immunoprecipitant was used to phosphorylate Rb protein to measure Cdk2 activity. The kinase assays were performed by immunoprecipitation with anti-Cdk2 antibodies (Santa Cruz Biotech) and Protein A sepharose beads (Amersham-Pharmacia Biotech) of active Cdk2 enzyme from 200 μg of total liver protein; non-specific proteins were removed by repetitive washes as described in Kiyokawa et al. (1996, Cell 85:721-732). Rb protein (Santa Cruz Biotech) was added with γ-{³²P}-ATP to the immunoprecipitated Cdk2 protein, which was bound to the Protein A sepharose beads. The kinase reaction was incubated for 30 minutes at 37° C. and one half of the Cdk2 kinase reaction was separated by SDS-PAGE and exposed to a phosphorimager screen. Quantitation of Cdk2 mediated Rb phosphorylation was performed with the Strom 860 phosphorimager and the ImageQuant program (Amersham-Pharmacia Biotech).

These kinase assays demonstrated that diminished S-phase levels of p27 protein were associated with elevated Cdk2 kinase activity in AdFoxM1B infected regenerating liver (FIG. 19B) compared to AdEmpty infected controls (FIG. 19A). The results suggest that AdFoxM1B infection of old-aged mice diminished nuclear expression of p27 protein in regenerating hepatocytes, which allowed complex formation of the active CyclinE-Cdk2 and Cyclin A-Cdk2 complexes required for S-phase progression.

Regenerating liver sections isolated at 28, 32, or 40 hours from AdEmpty infected 12-month old mice or AdFoxM1B infected 12-month old Balb/c mice were subjected to immunohistochemical staining with anti-Cdc25B antibodies (Santa Cruz, Calif.). Paraffin embedded tissues were cut into 5 μm sections, subjected to a 15 minute proteinase K antigen retrieval step (20 μg/mL Proteinase K, Invitrogen) in PBS at room temperature, rinsed with PBS, and stained with anti-Cdc25B antibodies. The immunohistochemical staining showed biphasic nuclear staining of Cdc25B phosphatase protein prior to and following S-phase (FIGS. 20D-F) in the AdFoxM1B infected mice. In contrast, low levels of either nuclear or perinuclear hepatocyte staining of Cdc25B protein were found at 28 hours post PHx in the AdEmpty infected old-aged liver (FIG. 20A). Furthermore, only perinuclear hepatocyte staining of the Cdc25B protein was found at later time points following PHx (FIGS. 20A-C). These results indicated that elevated levels of FoxM1B were associated with increases Cdc25B nuclear staining of regenerating hepatocytes, which is consistent with stimulation of M-phase progression in these cells.

### Example 11

Proliferation and Mitosis in Conditional FoxM1B Knockout Mice During Liver Regeneration

FoxM1B knockout mice die immediately after birth. Therefore, to examine the role of FoxM1B in adult liver regeneration, conditional FoxM1B knockout mice were generated using a triple-LoxP FoxM1B targeting vector to create a “Floxed” FoxM1B targeted locus (see FIG. 21 for a schematic diagram of the vector). Cre recombinase-mediated deletion of the FoxM1 genomic sequences spanning the two LoxP sites removes the entire winged helix DNA binding domain and the C-terminal transcriptional activation domain, thereby preventing expression of functional FoxM1 isoforms. Following standard electroporation and culture of mouse embryonic stem (ES) cells to select for homologous recombination (G418 and gangcyclovir), homologous recombinants were identified by Southern blotting of ES cell genomic DNA.

Mouse blastocysts were injected with the ES cells comprising the “Floxed” (fl/+) FoxM1B targeted allele, and chimeric mice with germ line transmission were selected. Viable mice homozygous for the “Floxed” (fl/fl) FoxM1B targeted allele were generated in this manner. Mice, either homozygous (fl/fl) or heterozygous (fl/+) for the FoxM1B (fl) allele, were verified by PCR amplification of mouse genomic DNA with primers that flanked the LoxP site. Breeding the albumin promoter Cre recombinase transgene into the FoxM1B (fl/fl) mouse genetic background allowed hepatocyte deletion of the FoxM1B locus within six weeks after birth, which was verified by Southern blot using liver genomic DNA.

The role of FoxM1B in hepatocyte proliferation was examined by performing liver regeneration studies with FoxM1B fl/fl and FoxM1B −/− mice in which the FoxM1B gene was deleted in hepatocytes by the albumin Cre recombinase transgene. Eight-week old FoxM1B −/− mice were subjected to partial hepatectomy (PHx) and their regenerating livers were harvested at different intervals between 24 and 52 hours following surgery (Wang et al., 2001, Proc. Natl. Acad. Sci. USA 98:11468-11473). Hepatocyte DNA synthesis was monitored by immunohistochemical staining of 5-bromo-2′-deoxyuridine (BrdU) incorporation into DNA as described above.

The FoxM1B fl/fl mice exhibited an 8-hour earlier expression of FoxM1B (at 32-hrs post PHx) in comparison to regenerating WT liver (Id.). Because FoxM1B is predominantly regulated at the post-transcriptional level, the LoxP neo construct at the 3′ end of the FoxM1B gene presumably stabilized its mRNA and thus enhanced induced FoxM1B levels. FoxM1B (fl/fl) mice exhibited a bifunctional S-phase peak in BrdU incorporation post PHx (FIG. 22A), while a significant reduction in DNA replication was observed in FoxM1B (−/−) regenerating livers (FIG. 22A). In addition, progression into mitosis was significantly reduced in regenerating hepatocytes of FoxM1B (−/−) mice as evidenced by the paucity of mitotic figures between 36 to 52 hours post PHx (FIG. 22B).

RNase protection assays were performed in duplicate to identify cell cycle regulatory genes, whose expression is diminished in regenerating liver of FoxM1B −/− mice (results shown in FIG. 23A). Minimal changes in cyclin D or cyclin E mRNA levels in regenerating liver of FoxM1B (−/−) mice were detected (FIG. 23A). However, Western blot analysis revealed elevated p21 protein levels in regenerating FoxM1B −/− hepatocytes compared to the FoxM1B fl/fl equaled controls (FIG. 23B). Since p21 protein inhibits cyclin/cdk activity, increased p21 protein levels provide an explanation for the decreases in DNA replication in regenerating FoxM1B −/− hepatocytes.

Diminished progression into mitosis of regenerating FoxM1B −/− livers is consistent with reduction in Cdc25B mRNA levels between 40 to 48 hour time points following the PHx operation. Western blot analysis with cdk-1 specific phospho-Tyrosine 15 antibodies demonstrated increased cdk-1 phosphorylation in FoxM1B deficient hepatocytes (FIG. 23C), a finding consistent with diminished levels of the Cdc25B phosphatase leading to reduced cdk1 activity (Nilsson et al., 2000, Prog. Cell Cycle Res. 4:107-114; Sebastian et al., 1993, Proc. Natl. Acad. Sci. USA 90:3521-3524; Trembley et al., 1996, Cell Growth Differ. 7:903-916). In support of diminished cdk1 activity, immunoprecipitation-kinase assays demonstrated that protein extracts from regenerating FoxM1B −/− hepatocytes displayed reduced cdk-1-dependent phosphorylation of the histone H1 substrate (FIG. 23C). Also, reduced cyclin A2, cyclin B1 and cdk1 levels were observed in FoxM1B −/−, but their expression was still increased during the cell cycle.

Collectively, these results suggested that FoxM1B regulates an essential activator of M-phase progression (Cdc25B) and mediated diminished p21 expression that facilitates entry into S-phase.

We also examined regenerating livers of Alb-Cre FoxM1B −/− mice at 7 days after PHx to determine whether reduction in cellular proliferation in regenerating Alb-Cre FoxM1B −/− hepatocytes caused deleterious effects. Paraffin embedded liver sections were stained with Hematoxylin and Eosin (FIGS. 24C-D) and 4′,6′-diamidino-2-phenylindole hydrochloride (DAPI) (FIGS. 24E-F). As expected, the number of hepatocytes in the regenerating FoxM1B −/− livers was half that of hepatocytes in the FoxM1B fl/fl livers (FIG. 24A). In contrast, the liver weight to body weight ratios for regenerating Alb-Cre FoxM1B −/− livers was higher than expected, suggesting that they underwent a compensatory increase in size (FIG. 24B). The histology of the regenerating liver sections demonstrated that Alb-Cre FoxM1B −/− hepatocytes displayed extensive hypertrophy at 7 days after PHx (FIGS. 24C-D). A TUNEL assay revealed that there was no increase in apoptosis (FIGS. 24G-H), suggesting that while FoxM1B mediated cell proliferation, it was not required for cell survival. Collectively, the data suggested that defective proliferation of regenerating Alb-Cre FoxM1B −/− liver resulted in hepatocyte hypertrophy, causing compensatory increase in liver size.

### Example 12

Regenerating FoxM1B −/− Livers Display Increased Levels of p21 and Diminished Cdc25A Protein Expression Leading to Decreased Cdk2 Activity

p21 expression was assayed in regenerating Alb-Cre FoxM1B −/− livers as follows. Western blot analysis performed as described herein was conducted using 50 μg of total liver protein from regenerating Alb-Cre FoxM1B −/− livers and blotting with anti-p21 antibodies (Calbiochem; 1:750). The results showed that the livers displayed increased p21 levels compared to the FoxM1B fl/fl control liver (FIG. 25A). Furthermore, immunohistochemical staining with anti-p21^{Cip1} antibodies (Calbiochem) of μm sections of paraffin-embedded regenerating liver tissues from these animals showed that regenerating FoxM1B fl/fl hepatocytes exhibited only a transient increase in nuclear p21 staining at 32 hours following PHx (FIGS. 25B-E), while Alb-Cre FoxM1B −/− hepatocytes displayed a sustained increase in nuclear p21 protein levels between 24 to 40 hours after PHx (FIGS. 25F-I).

Regenerating livers from p21−/− mice display increased Cdc25A expression and earlier nuclear localization of Cdc25A (Jaime et al., 2002, Hepatology 35:1063-1071). Since the regenerating livers of the Alb-Cre FoxM1B −/− mice demonstrate increased p21 protein levels, the possibility that the increased p21 protein might result in diminished protein expression of Cdc25A phosphatase required for Cdk2 activity was examined. Total liver protein from regenerating livers was prepared and 50 μg samples were subjected to Western blot analysis as described herein. Briefly, 50 μg of total liver protein from each sample were separated and transferred to Protran membrane (Schleicher and Schuell, Keene, N.H.). The membrane was stained with anti-Cdc25A antibodies (Santa Cruz Biotech, 1:200). The signal was amplified by biotin-conjugated anti-rabbit IgG (Bio-Rad, Hercules, Calif.) and detected with Enhanced Chemiluminescence Plus (ECL-plus, Amersham Pharmacia Biotech, Piscataway, N.J.). The results showed that regenerating Alb-Cre FoxM1B −/− livers displayed reduced Cdc25A protein levels prior to S-phase that initiated at 36 hours after PHx (FIG. 26A).

To examine Cdk2 kinase activity, regenerating liver protein extracts were prepared from Alb-Cre FoxM1B −/− mice and were immunoprecipitated with anti-Cdk2 antibodies. The immunoprecipitant was used to phosphorylate Rb protein to measure Cdk2 activity. The kinase assays were performed by immunoprecipitation of active Cdk2 enzyme from 200 μg of total liver protein with anti-Cdk2 antibodies (Santa Cruz Biotech) and Protein A sepharose beads (Amersham-Pharmacia Biotech), and non-specific proteins were removed by repetitive washes as described in Kiyokawa et al. (1996, Cell 85:721-732). Rb protein (Santa Cruz Biotech) was added with γ-{32P}-ATP to the immunoprecipitated Cdk2 protein, which was bound to the Protein A sepharose beads. The kinase reaction was incubated for 30 minutes at 37° C. and one half of the Cdk2 kinase reaction was separated by SDS-PAGE and exposed to a phosphorimager screen. Quantitation of Cdk2 mediated Rb phosphorylation was performed with the Storm 860 phosphorimager and the ImageQuant program (Amersham-Pharmacia Biotech).

These kinase assays demonstrated that less hyperphosphorylation of the Rb protein (FIG. 26B, indicated by *) occurred in the regenerating liver extracts from the Alb-Cre FoxM1B −/− mice compared with the FoxM1B fl/fl regenerating liver extracts. The results also showed that Cdk2 kinase activity was reduced in the Alb-Cre FoxM1B −/− mice. Active Cyclin A2-Cdk2 kinase complex is required to phosphorylate the cdhl subunit of ubiquitin-ligase anaphase-promoting complex (APC), which prevents APC-mediated degradation of Cyclin B at the end of S-phase and consequently allows Cyclin B accumulation to promote entry into mitosis (Harbour and Dean, 2000, Genes Dev. 14:2393-2409). Consistent with this concept, Western blot analysis with anti-Cyclin B1 antibodies showed delayed S-phase accumulation of Cyclin B1 protein between 32 and 40 hours after PHx in liver extracts from Alb-Cre FoxM1B −/− and FoxM1B fl/fl littermates (FIG. 26C). The decrease in Cdc25A protein expression and increased nuclear expression of p21 protein demonstrated in the experiments described above were consistent with reduced Cdk2 kinase activity necessary for S-phase progression.

### Example 13

FoxM1B Activates Transcription of the Cdc25B Promoter

In view of the decreased Cdc25B protein levels detected in Alb-Cre FoxM1B −/− mice, the effect of FoxM1B expression on Cdc25B transcription was determined as follows. U20S cells were transfected with 50 ng of CMV-FoxM1B (1-748) cDNA, CMV-FoxM1B (1-688) or CMV empty expression vectors and 1500 ng of luciferase plasmids containing the −200 base pairs of the mouse Cdc25B promoter using Fugene6 (Roche). Protein extracts were prepared from transfected U20S cells 24 hours following DNA transfection and used to measure luciferase enzyme activity using Dual-Luciferase Assay System (Promega) as described in Wang et al. (2002, J. Biol. Chem. 277:44310-44316). The results, presented as the mean fold induction of Cdc25B promoter activity±SD from two separate experiments in duplicate with the control (CMV empty) set at 1.0, indicated that FoxM1B protein stimulated expression of all three Cdc25B promoter regions (FIG. 27A). In contrast, no transcriptional activation of the Cdc25B promoter was found with a C-terminal mutant FoxM1B (1-688) with deleted sequences that are critical for transcriptional activation (FIG. 27A).

These results demonstrated that FoxM1B regulated transcription of Cdc25B phosphatase gene, whose expression is essential for activation of Cdk1-Cyclin B kinase and M-phase progression.

FIG. 27B is a diagram showing FoxM1B regulation of cell cycle genes. As demonstrated in FIG. 27B, FoxM1B regulates expression of cell cycle proteins that stimulate Cdk2 and Cdk1 activity, which are essential for entry into DNA replication and mitosis, respectively. Consistent with the findings described herein, FoxM1B protein levels are elevated in a number of tumor cell lines (Ye et al., 1997, Mol. Cell Biol. 17:1626-1641; Korver et al., 1997, Nucleic Acids Res. 25:1715-1719; Yao et al., 1997, J. Biol. Chem. 272:19,827-19,836) and in human basal cell carcinomas (Teh et al., 2002, Cancer Res. 62:4773-4780), suggesting that FoxM1B is required for cellular proliferation in human cancers.

### Example 14

The Effects of Growth Hormone on Expression and Localization of FoxM1B in the Liver

Two-month old WT and TG CD-1 mice were subjected to intraperitoneal (IP) injection of human growth hormone (Somatropin (Norditropin), Novo Nordisk Pharmaceuticals Inc., Princeton, N.J.; 5 μg per gram body weight) in vehicle buffer (2.2 mg glycine, 0.325 mg Disodium Phosphate Dihydrate (Na₂HPO4, 2H₂O), 0.275 mg Sodium Phosphate Dihydrate (NaH₂PO₄, 2H₂O), and 11 mg Mannitol per mL of solution). Liver tissue was harvested at various time intervals (from 0 to 3 hours) following growth hormone administration. Liver tissue was paraffin embedded used for immunohistochemical staining with the FoxM1B antibody. Immunohistochemical staining demonstrated that human growth hormone induced nuclear staining of FoxM1B protein in WT mice within one half hour of growth hormone administration (FIG. 28C-D compared to FIGS. 28A-B) and nuclear staining of FoxM1B protein persisted until the 3 hour time point (FIGS. 28E-H). Nuclear staining of the transgenic FoxM1B protein was induced by growth hormone between 30 minutes and 3 hours following IP administration to the TTR-FoxM1B transgenic mice (FIG. 29). No hepatic nuclear FoxM1B staining was found mouse WT and TG mouse controls injected with the growth hormone vehicle buffer alone (FIG. 28 panels A and B). These studies demonstrated that growth hormone alone is capable of inducing nuclear localization of FoxM1B protein without liver injury caused by PHx or CCl₄.

Reduced FoxM1B levels were found in regenerating liver of old Balb/c mice (12 month old) compared with young Balb/c mice (2 month old) (FIG. 30). The effect of growth hormone on hepatocyte proliferation and FoxM1B expression in old-aged mice was examined by administering growth hormone to 12 month-old Balb/c mice before and after partial hepatectomy (PHx). Human growth hormone (HGH) or phosphate buffered saline (PBS) was administered to old-aged (12 month-old) Balb/c mice by intraperitoneal (IP) injection (5 μg per gram body weight) one hour before PHx operation. The mice were also given IP injections of HGH or PBS every eight hours after the operations until the regenerating livers were harvested.

Mice were injected with BrdU as described above and their livers were harvested at various time intervals between 24 and 48 hours post-PHx. Portions of the liver tissues were used to prepare total RNA for RNase protection assays. Liver tissues were processed and liver sections were stained with anti-BrdU antibodies as described above. BrdU-stained hepatocytes and visible mitotic figures were counted as previously described (Wang et al., 2001, Proc. Natl. Acad. Sci. U.S.A. 98: 11468-11473). Regenerating hepatocyte DNA replication in regenerating liver of old-aged mice as measured by BrdU incorporation was similar to levels observed in regenerating livers of young (2 month-old) mice (FIG. 31A). Also, mitosis in the regenerating livers of old-aged mice was similar to mitosis in regenerating livers of young mice (FIG. 31B).

FoxM1B expression measured by RNase protection assays was elevated in the regenerating livers of old mice that received periodic HGH injections during the regeneration process (FIG. 30). In addition, HGH treatments restored expression of the FoxM1B target gene Cdc25B phosphatase to levels found in young regenerating livers as shown below.

These studies suggest that FoxM1B expression was stimulated by growth hormone in regenerating liver.

### Example 15

Growth Hormone Induces Nuclear Localization of FoxM1B Protein in Quiescent Liver Cells

Green fluorescent protein was fused in frame with FoxM1B amino acids 1 to 748 and the CMV promoter was used to drive expression of the GFP-FoxM1B fusion protein. The CMV-GFP-FoxM1B expression vector was delivered in 2.5 mL of saline via mouse tail vein injection. The technique has previously demonstrated transduction of DNA expression plasmids in 10% of hepatocytes in vivo. Livers from one group of transduced animals were harvested and processed as described above. A second group of mice transduced with the CMV-GFP-FoxM1B expression vector were given IP injections of HGH 45 minutes before their livers were harvested. Liver sections from both groups were examined under fluorescent microscope. GFP-FoxM1B resided in the cytoplasm of quiescent hepatocytes from animals not treated with HGH (FIG. 32C) while GFP-FoxM1B displayed nuclear localization in hepatocytes from the second group of mice (FIG. 32D) treated with HGH. As a control, a third group of mice were transduced with CMV-GFP-FoxM1B-NLS (NLS=SV40 Large T-antigen nuclear localization sequence) (FIG. 32B). The pattern of nuclear localization of GFP-FoxM1B induced by HGH was similar to localization of the dysregulated GFP-FoxM1B-NLS.

These results demonstrated that growth hormone was sufficient to induce nuclear localization of FoxM1B protein in quiescent hepatocytes.

### Example 16

Generation of Rosa26-FoxM1B Transgenic (TG) Mice

The −800 bp Rosa26 promoter region has been reported to drive expression of the green fluorescent protein (GFP) in every tissue tested in transgenic mice (Kisseberth, et al., 1999, Dev Biol. 214:128-38). In order to express FoxM1B in all tissues, the −800 bp Rosa26 promoter region was fused to a Transthyretin (TTR) minigene construct (Wu et al., 1996, Genes Dev. 10:245-260) containing the truncated human FoxM1B cDNA (SEQ ID NO: 1) inserted into the TTR second exon located adjacent to the SV40 virus transcriptional termination sequences (FIG. 33A). The 3′ untranslated region was removed from the FoxM1B cDNA, which regulates FoxM1B mRNA stability and allows high levels of transgene mRNA expression in quiescent or non-dividing adult liver tissue (Ye et al., 1999, Mol. Cell. Biol. 19:8570-8580).

Eight inbred FVB/n TG mouse lines containing the Rosa26-FoxM1B transgene construct were generated (FIG. 33A) (Kalinichenko et al, 2003, J. Biol. Chem. Jul 16). Expression of the construct in the mouse lines was verified by PCR screening of mouse tail genomic DNA using primers from the TTR transgene construct (Rausa et al., 2000, Mol. Cell Biol. 20:8264-8282; Wu et al., 1996, Genes Dev. 10:245-260; Yan et al., 1990, EMBO J. 9:869-878; Ye et al., 1999, Mol. Cell. Biol. 19:8570-8580). RNase protection assays (RPA) with total RNA isolated from adult wild type (WT) mouse tissues demonstrated that Foxm1b is abundantly expressed in thymus and testis, which contain a large population of proliferative cells (FIG. 33B). Lower levels of Foxm1b mRNA were found in spleen, lung, kidney, intestine and ovaries, which possess lower numbers of dividing cells (FIG. 33B). Quiescent adult TG liver exhibited stable expression of the FoxM1B transgene mRNA, which deleted 800 nucleotides of the 3′ untranslated terminal sequences, suggesting that they mediated FoxM1B mRNA degradation in non-dividing cells (Ye et al., 1999, Mol. Cell. Biol. 19:8570-8580). Because this 3′ end deletion stabilized the FoxM1B transgene mRNA in quiescent cells, transgene mRNA expression pattern in TG mouse tissues could be determined using RPA with the TTR minigene probe (FIG. 33A).

RPA demonstrated that none of the WT tissue RNAs produced an RNase resistant hybridization product of 310 nucleotides with the TTR transgene probe indicative of FoxM1B transgene expression (FIG. 33C). Rosa26 FoxM1B TG mouse line #10 exhibited the widest expression of the FoxM1B transgene in lung, liver, brain, thymus, heart, spleen, kidney, intestine, skeletal muscle, testis and skin (FIG. 33C). The Rosa26 FoxM1B TG mouse lines #15, 27, 31 and 41 expressed the FoxM1B transgene in almost all of these tissues, but they exhibited lower transgene levels in the liver (FIG. 33C). Transgenic lines #17 and #21 lacked expression of transgene in all of the tissues examined (FIG. 33C), whereas TG line #28 displayed undetectable expression of the FoxM1B transgene in kidney, skin, intestine and muscle (FIG. 33C).

### Example 17

Premature Expression of FoxM1B Accelerates the Onset of DNA Replication in Rosa26-FoxM1B TG Lung Following BHT Injury

The ubiquitously expressing Rosa26-FoxM1B TG mouse line #10 was selected to examine whether premature FoxM1B levels are sufficient to accelerate proliferation of different pulmonary cell-types following Butylated Hydroxytoluene (BHT) lung injury. A single intraperitoneal (IP) injection of BHT was given to either Rosa26-FoxM1B TG mice or their WT littermates and five mice were sacrificed at each of the indicated time points after BHT treatment (Kalinichenko et al, 2003, J. Biol. Chem. July 16). BHT mediated lung injury was characterized initially by extensive damage to the distal lung epithelial and endothelial cells, which are subsequently repaired by cellular proliferation between 2 and 7 days after BHT administration (Adamson et al., 1977, Lab Invest. 36:26-32; Marino et al., 1972, Proc. Soc. Exp. Biol. Med. 140:122-125). BHT (3,5-di-t-butyl-4-hydroxytoluene; Sigma, St Louis, Mo.) was dissolved in corn oil (Mazola) at concentration 30 mg/mL and a single intraperitoneal (IP) injection of BHT (300 mg/kg of body weight) was given to Rosa26-FoxM1B transgenic mice or their wild type littermates (Kalinichenko et al., 2001, Am. J. Physiol. Lung Cell Mol. Physiol. 280:L695-L704).

Three mice per time point were killed by CO₂ asphyxiation following BHT administration, which included 24, 26, 42, 48, 72 or 96 hours following BHT administration, lung tissue was weighed and used to prepare total RNA. Alternatively, lungs were inflated with 4% paraformaldehyde (PFA), fixed overnight in 4% PFA at 4° C. and then paraffin embedded as described previously (Kalinichenko et al., 2001, Dev. Biol. 235:489-506; Kalinichenko et al., 2001, Am. J. Physiol. Lung Cell Mol. Physiol. 280:L695-L7043). An intraperitoneal (IP) injection of a PBS solution containing 10 mg/mL of 5-bromo-2′-deoxyuridine (BrdU, Sigma; 50 μg/g body weight) was administered two hours prior to harvesting the injured lung tissue. Determination of the number of lung cells undergoing DNA synthesis was performed by monoclonal antibody (Roche) detection of BrdU incorporation in lungs (5 μm paraffin sections) at various hours following BHT injury using the microwave antigen retrieval method described previously (Ye et al., 1999, Mol. Cell. Biol. 19:8570-8580).

Immunohistochemical staining with the FoxM1B antibody demonstrated that the FoxM1B transgene protein was detected at 24 hours following BHT lung injury (FIGS. 34A-B) and its nuclear staining persisted throughout the TG lung repair process (FIG. 34C, E). In WT lungs nuclear staining of Foxm1b became detectable at 36 hours after BHT injury (FIG. 34D) and reached a maximum by the 42-hour time point (FIG. 34F).

The Rosa-26 FoxM1B TG lungs displayed a statistically significant increase in DNA replication (BrdU incorporation) between 32 and 48 hours following BHT injury compared to wild type (WT) littermates at similar time points (FIG. 35A). This is mainly evident at 42 hours following BHT injury when the Rosa26-FoxM1B lungs exhibited a 10-fold increase in BrdU incorporation compared to WT littermates (FIGS. 35A-C). There was no significant difference in BrdU incorporation between WT and Rosa26-FoxM1B lungs at 72 hours following BHT injury (FIGS. 35D and E).

To identify which cell types were stimulated to proliferate earlier in regenerating Rosa26-FoxM1B TG lungs, double immunofluorescent staining was performed with a BrdU monoclonal antibody along with antibodies specific to a marker protein expressed in a distinct pulmonary cell lineage. At 42 hours following BHT injury, a greater number of double positive BrdU and Surfactant protein B (SPB) Type 2 cells were found in regenerating TG lungs (FIGS. 36A-F; orange arrows) compared to WT lungs (FIGS. 36A-C, G). At 42 hours following BHT injury, only Rosa26-FoxM1B TG lungs exhibited double positive BrdU and isolectin B4 alveolar endothelial cells, which are distinguished morphologically from epithelial Type II cells by their small nuclei (FIGS. 36D-F, G-L; white arrows). In contrast, endothelial cell proliferation in peripheral WT lungs was not detected until 72 hours after BHT injury (FIGS. 36D-F, P—S). Furthermore, Rosa26-FoxM1B transgenic lungs displayed earlier BrdU staining in peribronchiolar smooth muscle cells, bronchial epithelial cells, and endothelial cells lining pulmonary arteries at 42 hours following BHT injury (FIGS. 36M-O). These results suggest that premature expression of the FoxM1B transgene protein caused earlier proliferation of epithelial, endothelial and smooth muscle cells after BHT lung injury.

### Example 18

Cotransfection of p19 ARF Inhibits FoxM1B Transcriptional Activity by Targeting it to the Nucleolus

Cotransfection assays have demonstrated that the p19 ARF (p19) protein targets the E2F transcription factor to the nucleolus for degradation and thus provides an additional mechanism to limit progression into S-phase (Martelli et al., 2001, Proc. Natl. Acad. Sci. USA 98:4455-4460). Similarly, p19 expression vector significantly diminished FoxM1B transcriptional activity in cotransfection assays using U2OS cells (FIG. 36A). Furthermore, Ras signaling was essential for FoxM1B transcriptional activity because transfection of a dominant negative (dn) Ras protein significantly inhibited FoxM1B dependent reporter expression, whereas transfection of a dnAkt/protein kinase B protein had little effect on FoxM1B function (FIG. 37A). Consistent with p19 mediated transcriptional inhibition of FoxM1B, cotransfection of the p19 vector with a construct encoding green fluorescent protein (GFP) fused to full length FoxM1B protein (FIGS. 37B-C) targeted FoxM1B to the nucleolus. Nucleolin protein colocalization confirmed that p19 mediates nuclear targeting of GFP-FoxM1B.

In contrast, the p19 protein was unable to change the nuclear localization of the transcriptionally inactive GFP-FoxM1B protein, which lacks its C-terminal 60 amino acids, suggesting that these FoxM1B sequences were critical for this response (FIG. 37D). dnRas diminished FoxM1B transcriptional activity (FIG. 37A), but retained FoxM1B protein in the nucleus (FIG. 37E). These studies demonstrated that similar to the E2F transcription factor, the p19 tumor suppressor protein targeted the FoxM1B protein to the nucleolus, thereby inhibiting FoxM1B transcriptional activity and providing a novel mechanism by which the p19 protein inhibited cell cycle progression.

### Example 19

Human Growth Hormone Treatment of Old-Aged Mice Increases Regenerating Hepatocyte Proliferation.

Growth hormone was administered to three young or old-aged Balb/c mice every eight hours for 40 hours (6 GH injections) and analyzed for hepatocyte proliferation at 41 hours following the first GH injection. Hepatocyte DNA replication in the GH treated mice was compared to untreated young and old-aged control mice as monitored by immunohistochemical detection of BrdU incorporation in liver sections as described herein. These studies demonstrated that GH injections were sufficient to induce hepatocyte proliferation in both young and old-aged mice without PHx (FIG. 38), but at lower levels compared to GH treated old-aged mice at 40 hours following PHx (FIG. 31A).

### Example 20

Human Growth Hormone Treatment of Old-Aged Mice Stimulates Regenerating Hepatocyte Expression of Foxm1b, Cdc25B and Cyclin B1

RNase protection assays and Western blot analysis were used to determine if growth hormone could affect expression of Foxm1b, Cdc25B, and Cyclin B1 in regenerating hepatocytes. For Western blot analysis, 50 μg of total protein extracts prepared from regenerating liver were separated on SDS-PAGE and transferred to Protran membrane (Schleicher & Schuell, Keene, N.H.). The signals from primary rabbit antibodies specific to Cdc25B (Santa Cruz Biotech, 1:200) proteins and mouse antibodies specific to either FoxM1B (1:5000) or β-actin (clone AC-15; Sigma; 1:3000 dilution) proteins were amplified by biotin conjugated anti-rabbit or anti-mouse IgG (Bio-Rad, Hercules, Calif.), and detected with Enhanced Chemiluminescence Plus (ECL-plus, Amersham Pharmacia Biotech, Piscataway, N.J.). To produce the mouse antibody against human FoxM1B protein, human FoxM1B C-terminal region (amino acid 365 to 748) was cloned into the p28A+ expression vector (Novagen) in frame with the Histidine (His) tagged epitope. The His tagged FoxM1B 365-748 protein was expressed in BL21 DE3 bacteria, protein extracts were made and the His tagged FoxM1B 365-748 protein was affinity purified on Nickel column (Novagen) using the manufacturer's protocol. Mice were immunized with the affinity purified His-tagged-human FoxM1B 365-748 protein and mouse serum was isolated by the University of Illinois at Urbana/Champaign Immunological Resource Center. Total RNA was prepared from mouse liver at indicated hours post PHx using RNA-STAT-60 (Tel-Test “B” Inc. Friendswood, Tex.) and was used for RNase protection assays (RPA) with antisense {α-³²P} UTP labeled probes specific to either the Foxm1b or Cdc25B genes.

RNase protection assays revealed that GH treatment of old-aged mice increased expression of Foxm1b and its target gene Cdc25B at 36 hours following PHx and that these increased levels were sustained throughout the period of hepatocyte proliferation (FIG. 39A). Western blot analysis with Foxm1b and Cdc25B antibodies demonstrated that GH administration to old-aged mice caused a more substantial increase in hepatic protein levels of Foxm1b and Cdc25B between 40 and 44 hours following PHx compared to similar regenerating liver points in untreated old-aged mice (FIG. 39B). Interestingly, untreated old-aged mice exhibited a transient increase in expression of both Foxm1b and Cdc25B proteins at 36 hours following PHx, but the increase in these proteins was not sustained at the later time points following surgery (FIG. 39B). Consistent with the ability of GH to induce hepatocyte DNA replication without PHx (FIG. 38), increased hepatocyte nuclear staining of Foxm1b protein was found in either young or old-aged mice treated with GH alone (FIGS. 40G-H). Hepatocyte nuclear staining of Foxm1b protein was undetectable in untreated mice. These results suggested that GH administration is sufficient to induce Foxm1b protein expression in absence of liver injury.

Furthermore, regenerating hepatocytes of growth hormone treated old-aged mice displayed a substantial increase in the nuclear staining of Foxm1b and Cdc25B proteins (FIGS. 40B and 39D). This is compared to undetectable hepatocyte nuclear levels of these proteins in untreated old-aged mice at 40 hours after PHx (FIGS. 40A and 39C). Moreover, more substantial nuclear staining of Cyclin B1 was observed in regenerating hepatocytes of GH treated old-aged mice compared to untreated old-aged mice (FIGS. 40E-F). Taken together, these studies demonstrate that GH treatment of old aged mice increased regenerating hepatocyte proliferation, expression and nuclear localization of Foxm1b, Cdc25B and Cyclin B1 proteins.

Although FoxM1B expression is induced greater than 40-fold during cellular proliferation (Wang et al., 2001, Proc Natl Acad Sci USA 98:11468-11473; Wang et al., 2002, J. Biol. Chem.277:44310-44316), its promoter activity is increased only 4-fold in response to serum stimulation (Korver et al., 1997, Genomics; 46:435-442), suggesting that proliferative signaling also stimulates Foxm1b levels through increased mRNA stability. Consistent with this finding, deletion of the terminal 972 nucleotides from the 3′end of the human FoxM1B cDNA resulted in stabilization of the FoxM1B transgene mRNA in quiescent mouse hepatocytes (Ye et al., 1999, Mol. Cell. Biol. 19:8570-8580). Our current studies suggested tht GH treatment of old-aged mice increases regenerating liver expression of Foxm1b, in part, through stabilization of Foxm1b mRNA. Furthermore, we found that the FoxM1B transgene protein was cytoplasmic in quiescent hepatocytes and proliferative signaling was required to induce hepatocyte nuclear translocation of the FoxM1B transgene protein following PHx (Ye et al., 1999, Mol. Cell. Biol. 19:8570-8580). Although untreated old aged regenerating liver expressed low levels of Foxm1b mRNA, nuclear staining of the Foxm1b protein was undetectable in these regenerating hepatocytes. In contrast, GH treatment of old aged mice upregulated Foxm1b mRNA and protein levels and also stimulated regenerating hepatocyte nuclear levels of Foxm1b protein, suggesting that GH signaling increased nuclear localization of the Foxm1b protein.

### Example 21

Regenerating Livers from GH Treated Old-Aged Mice Displayed Diminished Levels of Cdk Inhibitor p27^{Kip1} and Increased Expression of Cdc25A Protein.

Western blot analysis was used to analyze expression levels of p27 and Cdc25A in regenerating livers from growth hormone treated mice. Western blotting was carried out as described above. The antibodies used were primary rabbit antibodies specific to either p27^{kip1} (Cell Signaling, Berkeley, Calif.) or Cdc25A (Santa Cruz Biotech, 1:200). Young regenerating liver displayed only a transient increase in p27 protein levels at 36 hours after PHx and exhibited sustained expression of Cdc25A phosphatase protein throughout the period of hepatocyte proliferation (FIG. 41). Regenerating liver extracts from untreated old-aged mice exhibited a sustained increase in p27 protein levels and were unable to maintain high levels of Cdc25A protein beyond 36 hours post-PHx (FIG. 41). In contrast, regenerating liver extracts from GH treated old aged mice exhibited significant decrease in p27 protein levels and sustained expression of the Cdc25A phosphatase (FIG. 41). These studies demonstrated that GH treatment of old aged mice was capable of increasing regenerating hepatocyte DNA replication by altering expression of proteins that stimulate Cdk2 activity possibly through restoration of Foxm1b levels.

### Example 22

GH Treatment of Alb-Cre Foxm1b −/− Mice Failed to Restore Regenerating Hepatocyte Proliferation Resulting from Foxm1b Deficiency.

Hepatocyte DNA replication and mitosis were measured in either untreated or GH treated young Alb-Cre Foxm1b −/− mice at 40, 44 and 48 hours following PHx and compared regenerating hepatocyte proliferation with that of age-matched regenerating Foxm1b fl/fl liver (control).

These liver regeneration studies demonstrated that GH treatment of Alb-Cre Foxm1b −/− mice could not overcome the significant reduction in hepatocyte DNA replication or mitosis, and that hepatocyte proliferation levels were similar to those found in untreated regenerating Alb-Cre Foxm1b −/− livers (FIGS. 42A and 41B). Consistent with the essential role of Foxm1b in transcriptional regulation of the Cdc25B phosphatase gene, administering human GH failed to increase expression of Cdc25B in Foxm1b deficient regenerating livers (FIG. 42C). Regenerating Alb-Cre Foxm1b −/− hepatocytes displayed sustained increase in nuclear staining of Cdk inhibitor p21^{Cip1} (p21) protein (FIGS. 42G-I), whereas regenerating Foxm1b fl/fl hepatocytes exhibited only a transient increase in p21 nuclear staining (FIGS. 42D-F). Consistent with the role of Foxm1b in regulating p21 protein expression, GH treatment of Alb-Cre Foxm1b −/− mice was unable to overcome the persistent increase in regenerating hepatocyte nuclear staining of p21 protein (FIGS. 42J-L). Taken together, these liver regeneration studies indicated that Foxm1b was essential for GH to stimulate regenerating hepatocyte proliferation.

### Example 23

FoxM1B Transgene Protein is Immediately Translocated to the Nucleus Following PHx.

Previous studies demonstrated that deletion of the terminal 972 nucleotides from the 3′end of the human FoxM1B (HFH-11B) cDNA resulted in stabilization of the FoxM1B transgene mRNA in uninjured liver and that the FoxM1B transgene protein was cytoplasmic in quiescent hepatocytes (Ye et al., 1999, Mol. Cell. Biol. 19:8570-8580). Following partial hepatectomy (PHx), nuclear FoxM1B transgene protein was detected by 16 hours (hrs) following PHx, which was 16 hrs earlier than in regenerating wild type (WT) liver (Ye et al., 1999, Mol. Cell. Biol. 19:8570-8580). This premature nuclear localization of the FoxM1B transgene protein in regenerating TG liver caused an eight-hour acceleration of the onset of hepatocyte DNA replication and mitosis by stimulating earlier expression of cell cycle genes (Ye et al., 1999, Mol. Cell. Biol. 19:8570-8580).

In this study, the same TTR-FoxM1B transgenic (TG) mouse line was used to determine whether the FoxM1B transgene protein was translocated to the nucleus at early time points following PHx. WT and TTR-FoxM1B TG mice were subjected to PHx, sacrificed at 15 minutes (resected and remnant liver), and at 1, 2, 4, 6 and 8 hours following PHx and regenerating liver tissue was harvested. Regenerating liver was used to either prepare nuclear protein extracts for Western Blot analysis or paraffin embedded and sectioned for immunohistochemical staining to determine FoxM1B nuclear levels with an N-terminal FoxM1B antibody. Surprisingly, immunohistochemical staining with a FoxM1B antibody showed rapid hepatocyte nuclear translocation of FoxM1B transgene protein within 15 minutes after beginning PHx (FIG. 43B), whereas FoxM1B protein was cytoplasmic in quiescent hepatocytes without surgery (FIG. 43A). In contrast, only low levels of hepatocyte FoxM1B nuclear staining were found immediately following PHx in WT liver (FIGS. 43C-D), but this FoxM1B staining rapidly disappeared within 1 hr following PHx (FIGS. 44A and F). Furthermore, hepatocyte nuclear staining of the FoxM1B transgene protein persisted for the first 6 hrs after PHx and significantly diminished by 8 hrs post surgery (FIGS. 44B-E). In contrast, nuclear staining was undetectable in regenerating WT hepatocytes between 1 and 8 hrs following PHx (FIGS. 43F-J).

To measure FoxM1B nuclear protein, Western blot analysis was performed with nuclear extracts prepared from regenerating liver and an N-terminal specific FoxM1B antibody. This Western blot analysis demonstrated increased nuclear levels of the FoxM1B protein in either resected (Res) or remnant (Rem) TTR-FoxM1B TG liver compared to resected WT liver immediately following PHx (FIG. 45A). Furthermore, TG liver expressed the largest amount of nuclear FoxM1B protein within 15 minutes following PHx compared to regenerating TG and WT liver at 28 and 32 hrs following PHx (FIG. 45A). Western Blot analysis with nuclear extracts prepared from regenerating TG liver demonstrated abundant levels of FoxM1B transgene protein at 1, 2 and 4 hrs after PHx (FIG. 45B). Consistent with diminished nuclear staining of FoxM1B at 8 hrs following PHx (FIG. 44E), expression of nuclear FoxM1B protein was significantly diminished by the 8 hr time point (FIG. 45B). These studies demonstrated that only regenerating TG liver displayed significant nuclear levels of the FoxM1B protein and that these levels persisted throughout the initial 6 hrs following PHx.

Next, it was determined whether induction of the hepatic acute phase response caused hepatocyte nuclear translocation of the FoxM1B transgene protein. The initial stages of liver regeneration are dominated by an acute phase response, which involves the release of cytokines Tumor Necrosis Factor α (TNFα), Interleukin 6 (IL-6) and IL-1, which are critical for priming hepatocytes to respond to mitogenic signaling. Bacterial Lipopolysaccharides (LPS) bind to the LPS receptor on macrophages and induce secretion of TNFα that subsequently stimulates synthesis and secretion of IL-6, IL-1α, and IL-1β cytokines, which bind to their cognate hepatocyte receptors causing changes in expression of hepatic transcription factors and acute phase response genes. In order to induce the acute phase response, both TG and WT mice were subjected to IP injections of LPS, mice were sacrificed at either 1 or 2 hours after LPS injection and their livers were isolated.

These livers were used to either prepare nuclear extracts for Western blot analysis or processed for immunohistochemical staining to determine FoxM1B nuclear levels with a FoxM1B antibody. Approximately half of the TG hepatocytes displayed nuclear staining of the FoxM1B transgene protein within 1 hour of LPS injection (FIG. 46A). By 2 hours following LPS injection, nearly all of the TG hepatocytes exhibited nuclear FoxM1B staining (FIG. 46B). In contrast, no detectable FoxM1B nuclear staining was found in WT liver following LPS treatment (FIGS. 46C-D). Although nuclear FoxM1B protein was detected by immunohistochemical staining in a subset of TG hepatocytes at 1 hour following LPS injection, Western blot analysis was only sensitive enough to detect nuclear FoxM1B transgene protein by 2 hours following LPS treatment (FIG. 46E). These studies suggest that acute phase cytokines are sufficient to mediate nuclear translocation of the FoxM1B transgene protein, but only minimal WT hepatocyte expression of nuclear Foxm1b protein was found in response to LPS administration.

These studies demonstrates that the growth factors Hepatocyte Growth Factor (HGF), Epidermal Growth Factor (EGF) and Transforming Growth Faqctor α (TGFα) that are released in response to partial hepatectomy are sufficient to induce FoxM1B nuclear localization. Furthermore, these studies demonstrates that the cytokines Tumor Necrosis Factor a (TNFa), Interleukin 6 (IL-6), IL-1α and IL-1β that are released in response to partial hepatectomy and the acute phase response to LPS challenge are sufficient to induce FoxM1B nuclear localization. These studies imply that HGF, EGF, TGFα, IL-6, IL-1α, and IL-1β proteins could also be used to increase FoxM1B expression and nuclear localization.

It should be understood that the foregoing disclosure emphasizes certain specific embodiments of the invention and that all modifications or alternatives equivalent thereto are within the spirit and scope of the invention as set forth in the appended claims.

We claim:

1. A method of treating or preventing symptoms associated with aging comprising the step of inducing protein expression, nuclear localization, or both protein expression and nuclear localization of FoxM1B in a target cell.

2. The method of claim 1, wherein protein expression, nuclear localization, or both protein expression and nuclear localization of FoxM1B is induced by contacting the target cell with a growth factor or a cytokine.

3. The method of claim 2, wherein the growth factor is human growth hormone, hepatocyte growth factor, epidermal growth factor, transforming growth factor α or a growth factor that induces Ras-MAP kinase signaling.

4. The method of claim 2, wherein the cytokine is tumor necrosis factor α, interleukin 6 (IL-6), IL-1α, or IL-1β.

5. The method of claim 1, wherein the target cell expresses endogenous FoxM1B protein.

6. The method of claim 1, wherein the target cell comprises a recombinant nucleic acid construct comprising a nucleotide sequence that encodes a protein having an amino acid sequence identified by SEQ ID NO: 2 operatively linked to a control sequence, wherein target cells produce FoxM1B protein thereby.

7. The method of claim 6, wherein the nucleotide sequence is identified by SEQ ID NO: 1.

8. The method of claim 6, wherein the recombinant nucleic acid construct is a vector.

9. The method of claim 6, wherein the recombinant nucleic acid construct is a viral vector.

10. The method of claim 9, wherein the viral vector is an adenovirus vector, an adeno-associated virus vector, a retrovirus vector, herpes simplex virus vector, or vaccinia virus vector.

11. The method of claim 6, wherein the recombinant nucleic acid construct is delivered to the target cell within a liposome.

12. The method of claim 1, wherein the aging is premature aging.

13. The method of claim 12, wherein the premature aging is caused by a disease.

14. A method of preventing or amelioriating the effects of an age-related disease or age-related proliferation disorder in a patient comprising the step of inducing expression, nuclear localization, or both expression and nuclear localization of FoxM1B protein in a target cell.

15. The method of claim 14, wherein expression, nuclear localization, or both expression and nuclear localization of FoxM1B is induced by contacting the target cell with an effective amount of a growth factor or a cytokine.

16. The method of claim 15, wherein the growth factor is human growth hormone, hepatocyte growth factor, epidermal growth factor, transforming growth factor α, or a growth factor that induces Ras-MAP kinase signaling.

17. The method of claim 15, wherein the cytokine is tumor necrosis factor a, interleukin 6 (IL-6), IL-1α, or IL-1β.

18. The method of claim 14, wherein the target cell comprises a recombinant nucleic acid construct comprising a nucleotide sequence that encodes a protein having an amino acid sequence identified by SEQ ID NO: 2 operatively linked to a control sequence, wherein target cells produce FoxM1B protein thereby.

19. The method of claim 18, wherein the nucleotide sequence is identified by SEQ ID NO: 1.

20. The method of claim 18, wherein the recombinant nucleic acid construct is a vector.

21. The method of claim 20, wherein the recombinant nucleic acid construct is a viral vector.

22. The method of claim 21, wherein the viral vector is an adenovirus vector, an adeno-associated virus vector, a retrovirus vector, herpes simplex virus vector, or vaccinia virus vector.

23. The method of claim 18, wherein the recombinant nucleic acid construct is delivered to the target cell within a liposome.

24. A method of treating diseases or disorders associated with premature aging comprising the step of inducing expression, nuclear localization, or both expression and nuclear localization of FoxM1B protein in a target cell.

25. The method of claim 24, wherein the expression, nuclear localization, or both expression and nuclear localization of FoxM1B is induced by contacting the target cell with a growth factor or a cytokine.

26. The method of claim 25, wherein the growth factor is human growth hormone, hepatocyte growth factor, epidermal growth factor, transforming growth factor α, or a growth factor that induces Ras-MAP kinase signaling.

27. The method of claim 25, wherein the cytokine is tumor necrosis factor α, interleukin 6 (IL-6), IL-1α, or IL-1β.

28. The method of claim 24, wherein the target cell comprises a recombinant nucleic acid construct comprising a nucleotide sequence that encodes a protein having an amino acid sequence identified by SEQ ID NO: 2 operatively linked to a control sequence, wherein target cells produce FoxM1B protein thereby.

29. The method of claim 28, wherein the nucleotide sequence is identified by SEQ ID NO: 1.

30. The method of claim 28, wherein the recombinant nucleic acid construct is a vector.

31. The method of claim 30, wherein the recombinant nucleic acid construct is a viral vector.

32. The method of claim 31, wherein the viral vector is an adenovirus vector, an adeno-associated virus vector, a retrovirus vector, herpes simplex virus vector, or vaccinia virus vector.

33. The method of claim 28, wherein the recombinant nucleic acid construct is delivered to the target cell within a liposome.

34. The method of claim 24, wherein a full-length or less than full-length FoxM1B gene is introduced into the target cell prior to inducing expression and nuclear localization of FoxM1B protein.

35. A method of preventing or amelioriating the effects of lung injury comprising the step of inducing expression, nuclear localization, or both expression and nuclear localization of FoxM1B protein in a lung cell.

36. The method of claim 35, wherein the expression, nuclear localization, or both expression and nuclear localization of FoxM1B is induced by contacting the target cell with a growth factor or a cytokine.

37. The method of claim 36, wherein the growth factor is human growth hormone, hepatocyte growth factor, epidermal growth factor, transforming growth factor α, or a growth factor that induces Ras-MAP kinase signaling.

38. The method of claim 36, wherein the cytokine is tumor necrosis factor a, interleukin 6 (IL-6), IL-1α, or IL-1β.

39. The method of claim 35, wherein the target cell comprises a recombinant nucleic acid construct comprising a nucleotide sequence that encodes a protein having an amino acid sequence identified by SEQ ID NO: 2 operatively linked to a control sequence, wherein target cells produce FoxM1B protein thereby.

40. The method of claim 39, wherein the nucleotide sequence is identified by SEQ ID NO: 1.

41. The method of claim 39, wherein the recombinant nucleic acid construct is a vector.

42. The method of claim 41, wherein the recombinant nucleic acid construct is a viral vector.

43. The method of claim 42, wherein the viral vector is an adenovirus vector, an adeno-associated virus vector, a retrovirus vector, herpes simplex virus vector, or vaccinia virus vector.

44. The method of claim 39, wherein the recombinant nucleic acid construct is delivered to the target cell within a liposome.

45. The method of claim 35, wherein a full-length or less than full-length FoxM1B gene is introduced into the target cell prior to inducing expression and nuclear localization of FoxM1B protein.

46. A method of stimulating lung regeneration in a mammal, comprising the step of contacting lung cells in the mammal with growth factor or a cytokine, wherein the lung cells express FoxM1B protein.

47. The method of claim 46, wherein the lung cells comprise a recombinant nucleic acid construct comprising a nucleotide sequence that encodes a protein having an amino acid sequence identified by SEQ ID NO: 2 operatively linked to a control sequence into the lung cells, whereby the lung cells express FoxM1B protein.

48. The method of claim 47, wherein the nucleotide sequence is identified by SEQ ID NO: 1.

49. The method of claim 47, wherein the recombinant nucleic acid construct is a vector.

50. The method of claim 49, wherein the recombinant nucleic acid construct is a viral vector.

51. The method of claim 50, wherein the viral vector is an adenovirus vector, an adeno-associated virus vector, a retrovirus vector, herpes simplex virus vector, or vaccinia virus vector.

52. The method of claim 47, wherein the recombinant nucleic acid construct is delivered to the lung cell within a liposome.

53. The method of claim 46, wherein the mammal is a human.

54. A method of stimulating lung regeneration comprising the steps of:
a. isolating lung cells from a first mammal;
b. introducing a recombinant nucleic acid construct comprising a nucleotide sequence that encodes a protein having an amino acid sequence identified by SEQ ID NO: 2 operatively linked to a promoter sequence into the lung cells, whereby the lung cells express FoxM1B protein;
c. introducing the lung cells that express FoxM1B protein into a second mammal; and
d. administering to the second mammal an amount of a growth factor a cytokine sufficient to induce nuclear localization of the FoxM1B protein in the lung cells.

55. The method of claim 54, wherein the nucleotide sequence is identified by SEQ ID NO: 1.

56. The method of claim 54, wherein the lung cells expressing FoxM1B protein are reintroduced into first mammal, and the first mammal is treated with an amount of growth factor a cytokine sufficient to induce expression and nuclear localization of the FoxM1B protein in the lung cells.

57. The method of claim 56, wherein the recombinant nucleic acid construct is a vector.

58. The method of claim 57, wherein the recombinant nucleic acid construct is a viral vector.

59. The method of claim 58, wherein the viral vector is an adenovirus vector, an adeno-associated virus vector, a retrovirus vector, herpes simplex virus vector, or vaccinia virus vector.

60. The method of claim 56, wherein the recombinant nucleic acid construct is delivered to the lung cell within a liposome.

61. The method of claim 54, wherein the first mammal is a human and wherein the second mammal is a human.

62. A method of screening for compounds that can prevent or amelioriate the effects of an age-related disease or age-related proliferation disorder in a patient comprising the steps of:
a. contacting a plurality of cells that comprise a full-length FoxM1B gene or a less then full-length Foxm1b gene, wherein the cells do not express FoxM1B protein under conventional culture conditions, with a candidate compound;
b. assaying FoxM1B localization in the cells; and
c. identifying a candidate compound when FoxM1B is localized in the nuclei of cells contacted with the compound but not localized in the nuclei of cells not contacted with the compound.

63. The method of claim 62, wherein the less than full-length FoxM1B gene comprises a nucleotide sequence identified by SEQ ID NO: 1.

64. A method of screening for compounds that can prevent or amelioriate the effects of an age-related disease or age-related proliferation disorder in a patient comprising the steps of:
a. contacting a plurality of cells that comprise a full-length FoxM1B gene or a less than full-length FoxM1B gene, wherein the cells do not express FoxM1B protein under conventional culture conditions, with a candidate compound;
b. assaying expression of cyclin dependent kinase inhibitors p21^{Cip1} (p21) and p27^{Kip1} (p27), and mitosis promoting cdc25B phosphatase in the cells; and
c. identifying a candidate compound 1f p21 and p27 protein levels are decreased while cdc25B protein levels are increased in cells contacted with the compound compared with cells not contacted with the compound.

65. The method of claim 64, wherein the less than full-length FoxM1B gene comprises a nucleotide sequence identified by SEQ ID NO: 1.

66. A method of screening for compounds that can induce lung regeneration comprising the steps of:
a. contacting a plurality of cells that comprise a full-length FoxM1B gene or a less than full-length FoxM1B gene, wherein the cells do not express FoxM1B protein under conventional culture conditions, with a candidate compound;
b. assaying FoxM1B localization in the cells;
c. selecting a candidate compound when FoxM1B is localized in the nuclei of cells contacted with the compound but not localized in the nuclei of cells not contacted with the compound; and
d. identifying a compound as a compound that can induce lung regeneration when lung cells are induced to proliferate when contacted with the compound in vitro or in vivo.

67. The method of claim 66, wherein the less than full-length FoxM1B gene comprises a nucleotide sequence identified in SEQ ID NO: 1.

68. A method of screening for compounds that induce nuclear localization of FoxM1B protein, comprising the steps of:
a. contacting a cell with a compound, wherein the cell expresses a green fluorescent protein-FoxM1B (GFP-FoxM1B) fusion protein;
b. detecting localization of the GFP-FoxM1B protein in the cells; and
c. identifying a compound as a compound that induces FoxM1B localization if the GFP-FoxM1B protein is localized in the nuclei of the cells.

69. A method of screening for compounds that induce nuclear localization of FoxM1B protein, comprising the steps of:
a. contacting a transgenic mouse with a compound, wherein the cells of the transgenic mouse express a green fluorescent protein-FoxM1B (GFP-FoxM1B) fusion protein;
b. detecting localization of the GFP-FoxM1B protein in a cell removed from the mouse; and
c. identifying a compound as a compound that induces FoxM1B localization if the GFP-FoxM1B protein is localized in the nuclei of the cell that is removed from the mouse.

70. A method of treating or preventing symptoms associated with aging comprising the step of introducing a recombinant nucleic acid construct that comprises a nucleotide sequence identified by SEQ ID NO: 1 into target cells, thereby restoring proliferative potential of the target cells.

71. A method of preventing or amelioriating the effects of an age-related disease or age-related proliferation disorder in a patient comprising the step of introducing a recombinant nucleic acid construct that comprises a nucleotide sequence identified by SEQ ID NO: 1 into target cells, thereby restoring proliferative potential of the target cells.

72. A method of treating diseases or disorders associated with premature aging comprising the step of introducing a recombinant nucleic acid construct that comprises a nucleotide sequence identified by SEQ ID NO: 1 into a target cell, thereby restoring proliferative potential of the target cell.

73. A method of preventing or ameliorating the effects of lung injury comprising the step of introducing a recombinant nucleic acid construct that comprises a nucleotide sequence identified by SEQ ID NO: 1 into lung cells, thereby restoring proliferative potential of the lung cells.
